(19) United States
(12) Patent Application Publication
(10) Pub. No.: US 2021/0388108 A1
(43) Pub. Date: Dec. 16, 2021

(54) ANTIBODIES SPECIFIC FOR GLYCOSYLATED APOJ AND USES THEREOF

(71) Applicants: GLYCARDIAL DIAGNOSTICS, S.L., Barcelona (ES); Fundacio Institut De Recerca De L'Hospital De La Santa Creu l Sant Pau, Barcelona (ES); CONSEJO SUPERIOR DE INVESTIGACIONES CIENTIFICAS, Madrid (ES)

(72) Inventors: Lina BADIMON MAESTRO, Barcelona (ES); Judit CUBEDO RAFOLS, Barcelona (ES); Teresa PADRO CAPMANY, Barcelona (ES)

(21) Appl. No.: 17/287,777

(22) PCT Filed: Oct. 23, 2019
(86) PCT No.: PCT/EP2019/078855

§ 371 Date: Apr. 22, 2021

(30) Foreign Application Priority Data
Oct. 23, 2018 (EP) 18382752.6

(51) Int. Cl. C07K 16/30; G01N 33/68

(52) U.S. Cl. CPC C07K 16/30; G01N 33/6893; C07K 2317/24; G01N 2800/50; G01N 2440/38; G01N 2800/32; G01N 2333/775

(57) ABSTRACT

The invention relates to new antibodies against specific glycosylation sites within the ApoJ protein as well as their application thereof in the diagnosis and prognosis of ischemia and the determination of the risk of a recurrent ischemic event.

### FIELD OF THE INVENTION

The present invention is comprised within the field of biomedicine. It specifically relates to antibodies which specifically binds to glycosylated ApoJ and their uses in diagnosis and prognosis of ischemia.

### BACKGROUND OF INVENTION

Acute myocardial infarction (AMI) is one of the most frequent clinical manifestations of atherothrombosis and represents one of the major causes of death and disability worldwide. Due to the importance of early revascularization and the high risk of death, early diagnosis is essential. It is in this context where biomarkers arise as an important tool to complement clinical assessment and the 12-lead electrocardiogram (ECG) for the diagnosis, triage, risk stratification, and management of patients with suspected AMI.

Due to their potential direct implication in different stages of the development of the pathology, proteins represent an excellent target for biomarker search. Thus, until now, most of the proposed biomarkers for cardiovascular disease (CVD) progression and clinical event presentation are proteins involved in different phases of the development of the disease such as: lipid metabolism (Apo A-I), inflammation (CRP), cellular necrosis (troponins, CK-MB and myoglobin) or cardiac function (NT-proBNP), among others. However, the diagnosis and management of acute coronary syndromes (ACS) are based on clinical assessment, electrocardiogram findings and troponin levels, the only group of accepted biomarkers. The application of this protocol entails some limitations that make indispensable the search of new biomarkers to improve the management algorithm of patients with myocardial ischemia. Due to their structural role, cardiac troponins (cTn) are excellent markers of irreversible cell damage. However, cTns are not able to detect the ischemic event before its progression to full blown necrosis. Despite high-sensitivity cTn assays (hs-cTn) can detect minimum amounts of circulating cTns and this event has been suggested to be associated to the ischemic phase, a recent study in the swine model of MI has revealed that early cTn elevations are associated with myocyte apoptosis, implying that some type of cell death is needed for the release of cTn. In addition, current guidelines highlight the need of performing serial hs-cTn measurements to make an adequate triage of patients with acute chest pain. It has been described that in the early phase of AMI, a shift in the glycosylation profile of apolipoprotein J (Apo J), also known as clusterin, can be detected. This has led to the proposal of glycosylated ApoJ as a potential biomarker for AMI (Cubedo J. et al., Journal of Proteome Research 2011; 10:211-20).

In this context, the identification of specific biomarkers of ischemia able to map the ischemic event from its initial stages would be crucial to improve the current diagnostic algorithm of acute ischemic events.

### SUMMARY OF THE INVENTION

The authors of the invention have surprisingly found a new tool for the diagnosis and prognosis of ischemia, as well as for the determination of the risk of a recurrent ischemic event, based on the determination of the systemic levels of glycosylated ApoJ protein by means of the use of specific monoclonal antibodies against specific glycosylation sites within the ApoJ protein. Unexpectedly, as shown in the examples of the present document, the monoclonal antibodies targeting the different glycosylation sites in Apo J show improved discriminating ability of the presence of ischemia than lectins specifically recognising the N-glycans found in Apo J. These results are unexpected as it is generally known that highly glycosylated proteins are typically difficult targets for mAb generation, being limited by unsatisfactory affinity and low specificity.

Thus, in a first aspect the invention relates to an antibody which specifically binds glycosylated ApoJ but which does not bind non glycosylated ApoJ wherein.
(i) the antibody specifically recognizes an epitope which comprises a N-glycosylation site within ApoJ and wherein said glycosylation site comprises an Asn residue selected from the group consisting of the Asn residues at positions 86, 103, 145, 291, 317, 354 or 374 with respect to the ApoJ precursor sequence as defined in the NCO database entry with accession number NP_001822.3 or
(ii) the antibody specifically recognizes or has been generated using a peptide selected from the group consisting of SEQ ID NO: 118, 119, 120, 121, 122, 123 or 124, wherein the peptides are modified with N-acetylglucosamine residues at the Asn residues at position 5 in SEQ ID NO: 118, at position 5 in SEQ ID NO:119, position 5 a in SEQ ID NO:120, at position 6 in SEQ ID NO:121, at position 5 in SEQ ID NO:122, at position 5 in SEQ ID NO:123 or at position 5 in SEQ ID NO:124.

In a second aspect the invention relates to a polynucleotide encoding the antibody of the inventions as well as vectors and host cells.

In a third aspect the invention relates to a composition comprising at least two antibodies as defined in the first aspect of the invention.

In a fourth aspect the invention relates to a method for the determination of glycosylated Apo J in a sample comprising the steps of:
(i) Contacting the sample with an antibody according to the first aspect of the invention or with a composition according to the third aspect of the invention under conditions adequate for the formation of a complex between the antibody and the glycosylated Apo J present in the sample,
(ii) Determining the amount of complex formed in step (i).

In a fifth aspect the invention relates to a method for the diagnosis of ischemia or ischemic tissue damage in a subject comprising determining in a sample of said subject the levels of glycosylated Apo J using an antibody as defined in the first aspect of the invention, a composition according to the third aspect of the invention or using the method as defined in the fourth aspect of the invention, wherein decreased levels of glycosylated Apo J with respect to a reference value are indicative that the patient suffers ischemia or ischemic tissue damage.

In a sixth aspect the invention relates to a method for predicting the progression of ischemia in a patient having suffered an ischemic event or for determining the prognosis of a patient having suffered an ischemic event, comprising determining in a sample of said patient the levels of glycosylated Apo J using an antibody as defined in the first aspect of the invention, a composition according to the third aspect of the invention or using the method as defined in in the fourth aspect of the invention, wherein decreased levels of glycosylated Apo J with respect to a reference value are indicative that the ischemia is progressing or of a poor prognosis of the patient.

In a seventh aspect the invention relates to a method for determining the risk that a patient suffering from stable coronary disease suffers a recurrent ischemic event comprising determining in a sample of said patient the levels of glycosylated Apo J using an antibody as defined in the first aspect of the invention, a composition according to the third aspect of the invention or using the method as defined in the fourth aspect of the invention, wherein decreased levels of glycosylated Apo J with respect to a reference value are indicative that the patient shows an increased risk of suffering a recurrent ischemic event.

In an eighth aspect the invention relates to the use of an antibody according to any the first aspect of the invention or of a composition according to the third aspect of the invention for the diagnosis of ischemia or ischemic tissue damage in a patient, for determining the progression of ischemia in a patient having suffered an ischemic event, for the prognosis of a patient having suffered an ischemic event or for determining the risk that a patient suffering from stable coronary disease suffers a recurrent ischemic event.

### DESCRIPTION OF THE FIGURES

FIG. 1. Apo J protein sequence showing the signal peptide (amino acids 1-22), followed by the beta (amino acids 23-227) and alpha chains (amino acids 228-449). Monoclonal antibodies have been developed against the 7 different N-glycosylation sites of the Apo J protein sequence (86, 103, 145, 291, 317, 354 and 374) with glucosamine (GlcNAc) residues.

FIG. 2. Schematic diagram showing the methodological approach used to develop the specific monoclonal antibodies against the seven Apo J-GlcNAc glycosylation sites. Nine clones have been produced one for five of the target sites and two clones for two of them.

FIG. 3. Apo J-GlcNAc total levels and detection levels with different MAbs. Bar diagrams (mean±SEM) showing the intensity (optical density (OD) in arbitrary units (AU)) of Apo J-GlcNAc levels in serum samples of healthy controls and ischemia pre-AMI patients measured: with the lectin-based immunoassay detecting total Apo J-GlcNAc levels (A) and with specific antibodies targeting each independent Apo J-GlcNAc glycosylated residue (B-J). Specifically, the detection of Apo J-GlcNAc with MAbs against glycosylated residues 2 (clone Ag2G-17) and 6 (clone Ag6G-1) depicted the strongest decrease in Apo J-GlcNAc levels in AMI patients in the early ischemic phase.

FIG. 4: Percentage of decrease in Apo J-GlcNAc levels measured in cardiac ischemia: with the lectin-based immunoassay detecting total Apo J-GlcNAc levels (Apo J-GlcNAc Total levels) and with specific antibodies targeting each independent Apo J-GlcNAc glycosylated residue in serum samples of healthy controls and ischemia pre-AMI patients.

FIG. 5. C-statistics ROC analysis results of MAbs combinations. ROC curves showing the combinations of MAbs for the detection of different Apo J-GlcNAc glycosylated residues with a higher discriminating ability for the detection of ischemia.

FIG. 6: Dot blot binding assay of specific antibodies Ag2G17 and Ag6G11 targeting GlcNAc glycosylated Apo J showing the specificity of the antibodies to bind Apo J protein purified from human plasma and serum but not for other highly glycosylated proteins such as Albumin and Transferrin.

### DETAILED DESCRIPTION OF THE INVENTION

The authors of the invention disclose herewith a new methodology for the diagnosis and prognosis of ischemia based on the identification of the glycosylation pattern of the protein ApoJ with specific monoclonal antibodies against each of the glycosylation sites in ApoJ.

Unless otherwise defined, all technical terms used herein have the same meaning as commonly understood by one of ordinary skill in the art to which this invention belongs. The definitions provided herewith and in every other aspect of the invention are equally applicable to the whole invention.

Antibodies

In a first aspect the invention relates to an antibody which specifically binds glycosylated ApoJ but which does not bind non-glycosylated ApoJ, wherein.
(i) the antibody specifically recognizes an epitope which comprises a N-glycosylation site within ApoJ and wherein said glycosylation site comprises a Asn residues selected from the group consisting of the Asn residues at positions 86, 103, 145, 291, 317, 354 or 374 with respect to the ApoJ precursor sequence as defined in the NCBI database entry with accession number NP_001822.3 or
(ii) the antibody specifically recognizes or has been generated using a peptide selected from the group consisting of SEQ ID NO: 118, 119, 120, 121, 122, 123 or 124, wherein the peptides are modified with N-acetylglucosamine residues at the Asn residues at position 5 in SEQ ID NO: 118, at position 5 in SEQ ID NO:119, position 5 a in SEQ ID NO:120, at position 6 in SEQ ID NO:121, at position 5 in SEQ ID NO:122, at position 5 in SEQ ID NO:123 or at position 5 in SEQ ID NO:124.

The term “antibody”, as used herein, refers to an immunoglobulin molecule or according to some embodiments of the invention, a fragment of an immunoglobulin molecule which has the ability to specifically bind to an epitope of a molecule (“antigen”). Naturally occurring antibodies typically comprise a tetramer which is usually composed of at least two heavy (H) chains and at least two light (L) chains. Each heavy chain is comprised of a heavy chain variable domain (abbreviated herein as VH) and a heavy chain constant domain, usually comprised of three domains (CH1, CH2 and CH3). Heavy chains can be of any isotype, including IgG (IgG1, IgG2, IgG3 and IgG4 subtypes). Each light chain is comprised of a light chain variable domain (abbreviated herein as VL) and a light chain constant domain (CL). Light chains include kappa chains and lambda chains. The heavy and light chain variable domain is typically responsible for antigen recognition, while the heavy and light chain constant domain may mediate the binding of the immunoglobulin to host tissues or factors, including various cells of the immune system (e.g., effector cells) and the first component (C1 q) of the classical complement system. The VH and VL domains can be further subdivided into domains of hypervariability, termed “complementarity determining regions,” that are interspersed with domains of more conserved sequence, termed “framework regions” (FR). Each VH and VL is composed of three CDR Domains and four FR Domains arranged from amino-terminus to carboxy-terminus in the following order: FR1-CDR1-FR2-CDR2-FR3-CDR3-FR4. The variable domains of the heavy and light chains contain a binding domain that interacts with an antigen. Of particular relevance are antibodies and their epitope-binding fragments that have been “isolated” so as to exist in a physical milieu distinct from that in which it may occur in nature or that have been modified so as to differ from a naturally occurring antibody in amino acid sequence.

The term “antibody” comprises whole monoclonal antibodies or polyclonal antibodies, or fragments thereof, that retain one or more CDR regions, and includes human antibodies, humanised antibodies, chimeric antibodies and antibodies of a non-human origin.

“Monoclonal antibodies” are homogenous, highly specific antibody populations directed against a single site or antigenic “determinant”. “Polyclonal antibodies” include heterogeneous antibody populations directed against different antigenic determinants.

In a particular embodiment, the antibody of the invention is an antibody of non-human origin, preferably of murine origin. In preferred embodiment, the antibody of the invention is a monoclonal antibody. In another particular embodiment, the antibody of the invention is a polyclonal antibody.

In a preferred embodiment, the antibody of the invention is a human-rabbit chimeric antibody. In a still preferred embodiment the human-rabbit chimeric antibody comprises rabbit variable domains (Vλ, Vκ, and VH) linked to human constant domains (Ck and CH1), in particular rabbit Vλ/Vκ domains fused to human CK and rabbit VH domain fused to human CH1 of human IgG1.

It is well known that the basic structural unit of an antibody comprises a tetramer. Each tetramer is constituted by two identical pairs of polypeptide chains, each of which is composed by a light chain (25 KDa) and by a heavy chain (50-75 KDa). The amino-terminal region of each chain includes a variable region of about 100-110 or more amino acids, which is involved in antigen recognition. The carboxy-terminal region of each chain comprises the constant region that mediates the effector function. The variable regions of each pair of light and heavy chains form the binding site of the antibody. Therefore, an intact antibody has two binding sites. Light chains are classified as K or λ. Heavy chains are classified as γ, μ, α, δ and ε, and they define the isotype of the antibody as respectively IgG, IgM, IgA, IgD or IgE.

The variable regions of each pair of light and heavy chains form the binding site of the antibody. They are characterized by the same general structure constituted by relatively preserved regions called frameworks (FR) joined by three hyper-variable regions called complementarity determining regions (CDR) (Kabat et al., 1991, Sequences of Proteins of Immunological Interest, 5th ed., NIH Publication No. 91-3242, Bethesda, Md.; Chothia and Lesk, 1987, J Mol Biol 196:901-17). The term “complementarity determining region” or “CDR”, as used herein, refers to the region within an antibody where this protein complements an antigen's shape. Thus, CDRs determine the protein's affinity (roughly, bonding strength) and specificity for specific antigens. The CDRs of the two chains of each pair are aligned by the framework regions, acquiring the function of binding a specific epitope. Consequently, both the heavy chain and the light chain are characterized by three CDRs, respectively CDRH1, CDRH2, CDRH3 and CDRL1, CDRL2, CDRL3.

The CDR sequences can be determined according to conventional criteria, for example by means of the criteria of IgBLAST: http://www.ncbi.nlm.nih.gov/igblast/(Ye et al., 2013, Nucleic Acids Res 41 (Web Server issue: W34-40), by following the numbering provided by Kabat et al, Sequences of Proteins of Immunological Interest, 5th ed., Public Health Service, National Institutes of Health, Bethesda, Md. (1991), or by following the numbering provided by Chothia et al. (1989, Nature 342:877-83).

As used herein, the antibody of the invention encompasses not only full length antibodies (e.g., IgG), but also antigen-binding fragments thereof, for example, Fab, Fab′, F(ab′)2, Fv fragments, human antibodies, humanised antibodies, chimeric antibodies, antibodies of a non-human origin, recombinant antibodies, and polypeptides derived from immunoglobulins produced by means of genetic engineering techniques, for example, single chain Fv (scFv), diabodies, heavy chain or fragments thereof, light chain or fragment thereof, VH or dimers thereof, VL or dimers thereof, Fv fragments stabilized by means of disulfide bridges (dsFv), molecules with single chain variable region domains (Abs), minibodies, scFv-Fc, VL and VH domains and fusion proteins comprising an antibody, or any other modified configuration of the immunoglobulin molecule that comprises an antigen recognition site of a desired specificity. The antibody of the invention may also be a bispecific antibody. An antibody fragment may refer to an antigen binding fragment.

As used herein a “recombinant antibody” is an antibody that comprises an amino acid sequence derived from two different species or, or two different sources, and includes synthetic molecules, for example, an antibody that comprises a non-human CDR and a human framework or constant region. In certain embodiments, recombinant antibodies of the present invention are produced from a recombinant DNA molecule or synthesized.

The person skilled in the art will understand that the amino acid sequences of the antibodies of the invention can include one or more amino acid substitutions such that, even though the primary sequence of the polypeptide is altered, the capacity of the antibody to bind to glycosylated ApoJ is maintained. Said substitution can be a conservative substitution and is generally applied to indicate that the substitution of one amino acid with another amino acid with similar properties (for example, the substitution of glutamic acid (negatively charged amino acid) with aspartic acid would be a conservative amino acid substitution).

Amino acid sequence modification(s) of the antibody described herein are contemplated. For example, it may be desirable to improve the binding affinity and/or other biological properties of the antibody. Amino acid sequence variants of the antibody are prepared by introducing appropriate nucleotide changes into the antibody encoding nucleic acid, or by peptide synthesis. Such modifications include, for example, deletions from, and/or insertions into and/or substitutions of, residues within the amino acid sequences of the antibody. Any combination of deletion, insertion, and substitution is made to achieve the final construct, provided that the final construct possesses the desired characteristics. The amino acid changes may also alter post-translational processes of the protein, such as changing the number or position of glycosylation sites.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a peptide with an N-terminal methionyl residue or the antibody polypeptidic chain fused to a cytotoxic polypeptide. Other insertional variants of the molecule include the fusion to the N- or C-terminus of an enzyme, or a polypeptide which increases its serum half-life.

Another type of variant is an amino acid substitution variant. These variants have at least one amino acid residue in the molecule replaced by a different residue. The sites of greatest interest for substitution mutagenesis of antibodies s include the hypervariable regions, but FR alterations are also contemplated.

Another type of amino acid variant of the antibody alters the original glycosylation pattern of the antibody. By altering is meant deleting one or more carbohydrate moieties found in the molecule, and/or adding one or more glycosylation sites that are not present in it. Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of any of these tripeptide sequences in a polypeptide creates a potential glycosylation site. O-linked glycosylation refers to the attachment of one of the monosaccharides or monosaccharide derivatives N-aceylgalactosamine, galactose, or xylose to a hydroxyamino acid, most commonly serine or threonine, although 5-hydroxyproline or 5-hydroxy lysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

The affinity of an antibody for an antigen can be defined as the effectiveness of the antibody for binding such antigen. Antigen-antibody binding is a reversible binding and so, when both molecules are in dilution in the same solution after sufficient time, this solution reaches an equilibrium in which the concentrations of antigen-antibody complex (AgAb), free antigen (Ag) and free antibody (Ab) are constant. Therefore, the ratio [AgAb]/[Ag]*[Ab] is also a constant defined as association constant named Ka which can be used to compare the affinity of some antibodies for its respective epitope.

The common way to measure the affinity is to experimentally determine a binding curve. This involves measuring the amount of antibody-antigen complex as a function of the concentration of the free antigen. There are two common methods of performing this measurement: (i) the classical equilibrium dialysis using Scatchard analysis and (ii) the surface plasmon resonance method in which either antibody or antigen are bound to a conductive surface and binding of antigen or antibody respectively affects the electrical properties of this surface.

The capacity of the antibody of the invention to bind to the glycosylated ApoJ protein can be determined by a number of assays that are available in the art. Preferably, the binding specificity of monoclonal antibodies produced by a clone of hybridoma cells is determined by immunoprecipitation or by an in vitro binding assay, such as radioimmunoassay (RIA), enzyme-linked immunoabsorbent assay (ELISA), surface plasmon resonance or by immunofluorescent techniques such as immunohistochemistry (IHC), fluorescence microscopy or flow cytometry.

Often it is only necessary to determine relative affinities of two or more antibodies that join the same epitope, such as in the case of the antibody of the invention and a functional variant thereof. In this case a competitive assay can be performed in which a serial dilution of one of the antibodies is incubated with a constant quantity of a ligand, and the second antibody labelled with any suitable tracer is then added. After binding of this mAb and washing the non-bounded antibodies, the concentration of second antibody is measured and plotted in relation to concentrations of the first antibody and analysed with Scatchard method. An example is Tamura et al., J. Immunol. 163: 1432-1441 (2000).

The term “Apo J”, as used herein, refers to a polypeptide also known as “clusterin”, “testosterone-Repressed Prostate Message”, “Apolipoprotein J”, “complement-Associated Protein SP-40,40”, “complement cytolysis inhibitor”, complement Lysis Inhibitor”, “sulfated glycoprotein”, “Ku70-Binding Protein”, “NA1/NA2”, “TRPM-2”, “KUB1”, “CLI”. Human Apo J is the polypeptide provided under accession number P10909 in the UniProtKB/Swiss-Prot database (Entry version 212 of 12 Sep. 2018).

The term “glycosylated” generally refers to any protein having covalently attached oligosaccharide chains.

The term “glycosylated ApoJ” or “Apo J containing GlcNAc residues”, as used herein, refers to any Apo J molecule containing at least one repeat of N-acetylglucosamine (GlcNAc) in at least one glycan chain, although typically, the Apo J will contain at least one N-acetylglucosamine in each glycan chain. In one embodiment, the glycosylated ApoJ contains an N-glycan at a single Asn residue selected from the group consisting of the Asn residues at positions 86, 103, 145, 291, 317, 354 or 374 with respect to the ApoJ preproprotein sequence as defined in the NCBI database entry with accession number NP_001822.3 (release of Sep. 23, 2018). In another embodiment, the glycosylated ApoJ contains N-glycans at every N-glycosylation site within ApoJ, i.e. at each Asn at positions 86, 103, 145, 291, 317, 354 and 374 with respect to the ApoJ preproprotein sequence as defined in the NCBI database entry with accession number NP_001822.3 (release of Sep. 23, 2018).

“Apo J containing GlcNAc residues” include Apo J molecules containing at least one GlcNAc residue in high-mannose N-glycans, complex-type N-glycans, hybrid oligosaccharides N-glycans or O-glycans. Depending on the type of N-glycans, the GlcNAc may be found directly attached to the polypeptide chain or in a distal position in the N-glycan.

The term “GlcNAc” or “N-acetyl glucosamine” refers to a derivative of glucose resulting from the amidation of glucosamine by acetic acid and having the general structure:

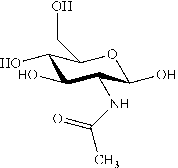

In one embodiment, the Apo J-containing GlcNAc residues contains two residues of GlcNAc and is referred herein as (GlcNAc)₂. Apo J molecules containing (GlcNAc)₂ residues include molecules wherein the (GlcNAc)₂ is found in high-mannose N-glycans, in complex-type N-glycans, in hybrid oligosaccharides N-glycans or in 0-glycans. Depending on the type of N-glycans, the (GlcNAc)₂ may be found directly attached to the polypeptide chain or in a distal position in the N-glycan.

In a preferred embodiment, the “Apo J-containing GlcNAc residues” is substantially free of other types of N-linked or O-linked carbohydrates. In one embodiment, the “Apo J containing GlcNAc residues” does not contain N-linked or O-linked α-mannose residues. In another embodiment, the “Apo J-containing GlcNAc residues” does not contain N-linked or O-linked α-glucose residues. In yet another embodiment, the “Apo J-containing GlcNAc residues” does not contain N-linked or O-linked α-mannose residues or N-linked or O-linked α-glucose residues.

The term “non-glycosylated ApoJ”, as used herein, refers to ApoJ wherein none of the Asn residues at positions 86, 103, 145, 291, 317, 354 or 374 in the ApoJ polypeptide with respect to the ApoJ preproprotein sequence as defined in the NCBI database entry with accession number NP_001822.3 (release of Sep. 23, 2018) are glycosylated.

The term “binding”, “bond” or “binds” according to the invention refers to the interaction between affinity binding molecules or specific binding pairs as a result of non-covalent bonds, such as, but not limited to, hydrogen bonds, hydrophobic interactions, van der Waals bonds, ionic bonds or a combination of the above. The term “binding pair” does not involve any particular size of any other technical structural characteristic other than that said binding pair can interact and bind to the other member of the binding pair resulting in a conjugate wherein the first and second components are bound to each other by means of the specific interaction between the first and second member of a binding pair. Within the context of the present invention binding pair includes any type of immune interaction such as antigen/antibody, antigen/antibody fragment or hapten/anti-hapten.

The term “specifically binds”, “specific binding” or specifically recognizes”, when used in the present invention to refer to the binding of an antibody or a fragment thereof to a glycosylated form of Apo J, is understood as the capacity of the antibody or fragment thereof to bind specifically to glycosylated form of Apo J by means of the existence of complementarity between the three-dimensional structures of the two molecules with a substantially higher affinity for non-specific binding such that the binding between said antibody or fragment thereof and glycosylated form of Apo J preferably takes place before the binding of any of said molecules with respect to the other molecules present in the reaction mixture. This results in that the antibody or fragment thereof does not cross-react with other glycans which may or may not be present in the Apo J molecule. Cross-reactivity of the antibody or fragment thereof may be tested, for example, by assessing binding of said antibody or fragment thereof under conventional conditions to the glycan of interest as well as to a number of more or less (structurally and/or functionally) closely related glycan. Only if the antibody or fragment thereof binds to the glycan of interest but does not or does not essentially bind to any of the other glycans is considered specific for the glycan of interest. For instance, a binding can be considered specific if the binding affinity between the antibody and the glycosylated Apo J has a dissociation constant (KD) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M or less than 10⁻¹⁵ M.

In one embodiment, the antibody which specifically recognizes an epitope which comprises the N-glycosylation site within Apo J at positions 86 with respect to the Apo J precursor sequence as defined in the NCBI database entry with accession number NP_001822.3 does not substantially bind to one, more or any epitopes which comprise an N-glycosylation site within Apo J at positions 103, 145, 291, 317, 354 or 374.

In one embodiment, the antibody which specifically recognizes an epitope which comprises the N-glycosylation site within Apo J at position 103 with respect to the Apo J precursor sequence as defined in the NCBI database entry with accession number NP_001822.3 does not substantially bind to one, more or any epitopes which comprise an N-glycosylation site within Apo J at positions 86, 145, 291, 317, 354 or 374.

In one embodiment, the antibody which specifically recognizes an epitope which comprises the N-glycosylation site within Apo J at position 145, with respect to the Apo J precursor sequence as defined in the NCBI database entry with accession number NP_001822.3 does not substantially bind to one, more or any epitopes which comprise an N-glycosylation site within Apo J at positions, 86, 103, 291, 317, 354 or 374.

In one embodiment, the antibody which specifically recognizes an epitope which comprises the N-glycosylation site within Apo J at position 291 with respect to the Apo J precursor sequence as defined in the NCBI database entry with accession number NP_001822.3 does not substantially bind to one, more or any epitopes which comprise an N-glycosylation site within Apo J at positions 86, 103, 145, 317, 354 or 374.

In one embodiment, the antibody which specifically recognizes an epitope which comprises the N-glycosylation site within Apo J at position 317 with respect to the Apo J precursor sequence as defined in the NCBI database entry with accession number NP_001822.3 does not substantially bind to one, more or any epitopes which comprise an N-glycosylation site within ApoJ at positions 86, 103, 145, 291, 354 or 374.

In one embodiment, the antibody which specifically recognizes an epitope which comprises the N-glycosylation site within Apo J at position 354 with respect to the Apo J precursor sequence as defined in the NCBI database entry with accession number NP_001822.3 does not substantially bind to one, more or any epitopes which comprise an N-glycosylation site within Apo J at positions 86, 103, 145, 291, 317 or 374.

In one embodiment, the antibody which specifically recognizes an epitope which comprises the N-glycosylation site within Apo J at position 374 with respect to the Apo J precursor sequence as defined in the NCBI database entry with accession number NP_001822.3 does not substantially bind to one, more or any epitopes which comprise an N-glycosylation site within Apo J at positions 86, 103, 145, 291, 317 or 354.

In one embodiment, the antibody according to the invention which shows specific binding to an epitope which comprises an N-glycosylation site within ApoJ does not substantially bind other epitopes in Apo J that comprise a N-glycosylation site and/or does not substantially bind N-glycosylated polypeptides other than ApoJ.

In another embodiment, the antibody which specifically recognizes the peptide of SEQ ID NO: 118 modified with an N-acetylglucosamine residue at the Asn residue at position 5 does not substantially recognize one, more or any of the peptides of SEQ ID NO:119, 120, 121, 122, 123 or 124, wherein the peptides are modified with N-acetylglucosamine residues at the Asn residues at position 5 in SEQ ID NO:119, position 5 a in SEQ ID NO:120, at position 6 in SEQ ID NO:121, at position 5 in SEQ ID NO:122, at position 5 in SEQ ID NO:123 or at position 5 in SEQ ID NO:124.

In another embodiment, the antibody which specifically recognizes the peptide of SEQ ID NO: 119 modified with an N-acetylglucosamine residue at the Asn residue at position 5 does not substantially recognize one, more or any of the peptides of SEQ ID NO:118, 120, 121, 122, 123 or 124, wherein the peptides are modified with N-acetylglucosamine residues at the Asn residues at position 5 in SEQ ID NO:118, position 5 a in SEQ ID NO:120, at position 6 in SEQ ID NO:121, at position 5 in SEQ ID NO:122, at position 5 in SEQ ID NO:123 or at position 5 in SEQ ID NO:124.

In another embodiment, the antibody which specifically recognizes the peptide of SEQ ID NO: 120 modified with an N-acetylglucosamine residue at the Asn residue at position 5 does not substantially recognize one, more or any of the peptides of SEQ ID NO:118, 119, 121, 122, 123 or 124, wherein the peptides are modified with N-acetylglucosamine residues at the Asn residues at position 5 in SEQ ID NO:118, position 5 a in SEQ ID NO:119, at position 6 in SEQ ID NO:121, at position 5 in SEQ ID NO:122, at position 5 in SEQ ID NO:123 or at position 5 in SEQ ID NO:124.

In another embodiment, the antibody which specifically recognizes the peptide of SEQ ID NO: 121 modified with an N-acetylglucosamine residue at the Asn residue at position 6 does not substantially recognize one, more or any of the peptides of SEQ ID NO:118, 119, 120, 122, 123 or 124, wherein the peptides are modified with N-acetylglucosamine residues at the Asn residues at position 5 in SEQ ID NO:118, position 5 a in SEQ ID NO:119, at position 5 in SEQ ID NO:120, at position 5 in SEQ ID NO:122, at position 5 in SEQ ID NO:123 or at position 5 in SEQ ID NO:124.

In another embodiment, the antibody which specifically recognizes the peptide of SEQ ID NO: 122 modified with an N-acetylglucosamine residue at the Asn residue at position 5 does not substantially recognize one, more or any of the peptides of SEQ ID NO:118, 119, 120, 121, 123 or 124, wherein the peptides are modified with N-acetylglucosamine residues at the Asn residues at position 5 in SEQ ID NO:118, position 5 a in SEQ ID NO:119, at position 5 in SEQ ID NO:120, at position 6 in SEQ ID NO:121, at position 5 in SEQ ID NO:123 or at position 5 in SEQ ID NO:124.

In another embodiment, the antibody which specifically recognizes the peptide of SEQ ID NO: 123 modified with an N-acetylglucosamine residue at the Asn residue at position 5 does not substantially recognize one, more or any of the peptides of SEQ ID NO:118, 119, 120, 121, 122 or 124, wherein the peptides are modified with N-acetylglucosamine residues at the Asn residues at position 5 in SEQ ID NO:118, position 5 a in SEQ ID NO:119, at position 5 in SEQ ID NO:120, at position 6 in SEQ ID NO:121, at position 5 in SEQ ID NO:122 or at position 5 in SEQ ID NO:124.

In another embodiment, the antibody which has been generated using the peptide of SEQ ID NO: 118 wherein the peptide is modified with an N-acetylglucosamine residue at position 5 in SEQ ID NO: 118 does not substantially recognize one, more or any of the peptides of 119, 120, 121, 122 or 124, wherein the peptides are modified with N-acetylglucosamine residues at the Asn residues at position 5 a in SEQ ID NO:119, at position 5 in SEQ ID NO:120, at position 6 in SEQ ID NO:121, at position 5 in SEQ ID NO:122, at position 5 in SEQ ID NO:123 or at position 5 in SEQ ID NO:124.

In another embodiment, the antibody which has been generated using the peptide of SEQ ID NO: 119 wherein the peptide is modified with an N-acetylglucosamine residue at the Asn residue at position 5 does not substantially recognize one, more or any of the peptides of SEQ ID NO:118, 120, 121, 122 or 124, wherein the peptides are modified with N-acetylglucosamine residues at the Asn residues at position 5 in SEQ ID NO:118, at position 5 in SEQ ID NO:120, at position 6 in SEQ ID NO:121, at position 5 in SEQ ID NO:122, at position 5 in SEQ ID NO:123 or at position 5 in SEQ ID NO:124.

In another embodiment, the antibody which has been generated using the peptide of SEQ ID NO: 120, wherein the peptide is modified with an N-acetylglucosamine residue at position 5 a in SEQ ID NO:120 does not substantially recognize one, more or any of the peptides of SEQ ID NO:118, 119, 121, 122 or 124, wherein the peptides are modified with N-acetylglucosamine residues at the Asn residues at position 5 in SEQ ID NO:118, position 5 a in SEQ ID NO:119, at position 6 in SEQ ID NO:121, at position 5 in SEQ ID NO:122, at position 5 in SEQ ID NO:123 or at position 5 in SEQ ID NO:124.

In another embodiment, the antibody which has been generated using the peptide of SEQ ID NO: 121 wherein the peptide is modified with an N-acetylglucosamine residue at position 6 in SEQ ID NO:121 does not substantially recognize one, more or any of the peptides of SEQ ID NO:118, 119, 120, 122 or 124, wherein the peptides are modified with N-acetylglucosamine residues at the Asn residues at position 5 in SEQ ID NO:118, position 5 a in SEQ ID NO:119, at position 5 in SEQ ID NO:120 at position 5 in SEQ ID NO:122, at position 5 in SEQ ID NO:123 or at position 5 in SEQ ID NO:124.

In another embodiment, the antibody which has been generated using the peptide of SEQ ID NO: 122, wherein the peptide is modified with an N-acetylglucosamine residue at position 5 does not substantially recognize one, more or any of the peptides of SEQ ID NO:118, 119, 120, 121, 124, wherein the peptides are modified with N-acetylglucosamine residues at the Asn residues at position 5 in SEQ ID NO:118, position 5 a in SEQ ID NO:119, at position 5 in SEQ ID NO:120, at position 6 in SEQ ID NO:121, at position 5 in SEQ ID NO:123 or at position 5 in SEQ ID NO:124.

In another embodiment, the antibody which has been generated using the peptide of SEQ ID NO: 123 wherein the peptide is modified with an N-acetylglucosamine residue at position 5 does not substantially recognize one, more or any of the peptides of SEQ ID NO:118, 119, 120, 121, 122 or 124, wherein the peptides are modified with N-acetylglucosamine residues at the Asn residues at position 5 in SEQ ID NO:118, position 5 a in SEQ ID NO:119, at position 5 in SEQ ID NO:120, at position 6 in SEQ ID NO:121, at position 5 in SEQ ID NO:122 or at position 5 in SEQ ID NO:124.

In another embodiment, the antibody which has been generated using the peptide of SEQ ID NO: 124 wherein the peptide is modified with an N-acetylglucosamine residue at position 5 does not substantially recognize one, more or any of the peptides of SEQ ID NO:118, 119, 120, 121, 122 or 123, wherein the peptides are modified with N-acetylglucosamine residues at the Asn residues at position 5 in SEQ ID NO:118, position 5 a in SEQ ID NO:119, at position 5 in SEQ ID NO:120, at position 6 in SEQ ID NO:121, at position 5 in SEQ ID NO:122 or at position 5 in SEQ ID NO:123.

In another embodiment, the antibodies according to the invention do not substantially bind O-linked glycans, more preferably O-linked GlaNAc.

In additional embodiments, the antibody which specifically recognizes an epitope which comprises a N-glycosylation site within Apo J and wherein said glycosylation site comprises a Asn residues selected from the group consisting of the Asn residues at positions 86, 103, 145, 291, 317, 354 or 374 with respect to the Apo J precursor sequence as defined in the NCO database entry with accession number NP_001822.3 invention do not substantially bind O-linked glycans, more preferably O-linked GlaNAc.

In additional embodiments, the antibody which specifically recognizes or has been generated using a peptide selected from the group consisting of SEQ ID NO: 118, 119, 120, 121, 122, 123 or 124, wherein the peptides are modified with N-acetylglucosamine residues at the Asn residues at position 5 in SEQ ID NO: 118, at position 5 in SEQ ID NO:119, position 5 a in SEQ ID NO:120, at position 6 in SEQ ID NO:121, at position 5 in SEQ ID NO:122, at position 5 in SEQ ID NO:123 or at position 5 in SEQ ID NO:124 invention do not substantially bind O-linked glycans, more preferably O-linked GlaNAc.

In another embodiment, the antibodies according to the invention do not substantially bind N-acetlygalactosamine or an epitope which contains N-acetlygalactosamine and not N-acetylglucosamine.

In additional embodiments, the antibody which specifically recognizes an epitope which comprises a N-glycosylation site within Apo J and wherein said glycosylation site comprises a Asn residues selected from the group consisting of the Asn residues at positions 86, 103, 145, 291, 317, 354 or 374 with respect to the Apo J precursor sequence as defined in the NCO database entry with accession number NP_001822.3 invention do not substantially bind N-acetlygalactosamine or an epitope which contains N-acetlygalactosamine and not N-acetylglucosamine.

In additional embodiments, the antibody which specifically recognizes or has been generated using a peptide selected from the group consisting of SEQ ID NO: 118, 119, 120, 121, 122, 123 or 124, wherein the peptides are modified with N-acetylglucosamine residues at the Asn residues at position 5 in SEQ ID NO: 118, at position 5 in SEQ ID NO:119, position 5 a in SEQ ID NO:120, at position 6 in SEQ ID NO:121, at position 5 in SEQ ID NO:122, at position 5 in SEQ ID NO:123 or at position 5 in SEQ ID NO:124 invention do not substantially bind N-acetlygalactosamine or an epitope which contains N-acetlygalactosamine and not N-acetylglucosamine.

In another embodiment, the antibodies according to the invention do not substantially bind an epitope containing the sequence of SEQ ID NO:167 (TKLKELPGVCNETMMALWEE) wherein said epitope comprises a N-glycosylation at position 11.

In another embodiment, the antibody according to the invention which specifically recognizes an epitope which comprises the N-glycosylation site within Apo J at position 103 with respect to the Apo J precursor sequence as defined in the NCBI database entry with accession number NP_001822.3 invention do not substantially bind an epitope containing the sequence of SEQ ID NO:167 wherein said epitope comprises a N-glycosylation at position 11.

In additional embodiments, the antibody which specifically recognizes or has been generated using the peptide of SEQ ID NO: 119 wherein the peptide is modified with an N-acetylglucosamine residue at the Asn residue at position 5 in SEQ ID NO:119 do not substantially bind an epitope containing the sequence of SEQ ID NO:167 wherein said epitope comprises a N-glycosylation at position 11.

The capacity of the binding agents according to the invention, and in particular of the antibody or antibody fragment as described herein, to bind to the glycosylated ApoJ protein can be determined by a number of assays that are well known in the art. Preferably, the binding capacity of the binding agents is determined by immunoprecipitation or by an in vitro binding assay, such as radioimmunoassay (RIA), enzyme-linked immunoabsorbent assay (ELISA), surface plasmon resonance or by immunofluorescent techniques such as immunohistochemistry (IHC), fluorescence microscopy or flow cytometry.

In a preferred embodiment the antibody of the invention specifically binds to a glycosylated Apo J which is a glycosylated Apo J containing N-acetylglucosamine (GlcNAc) residues or to a glycosylated Apo J containing N-acetylglucosamine (GlcNAc) and sialic acid residues.

The term “Apo J-containing GlcNAc and sialic acid residues”, as used herein, refers to any Apo J molecule containing at least one repeat of N-acetyl-glucose in its glycan chain and at least one repeat of sialic acid residue.

The term “sialic acid”, as used herein, refers to the monosaccharide known as N-acetylneuraminic acid (Neu5Ac) and having the general structure

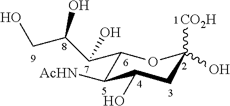

In one embodiment, the Apo J contains two residues of GlcNAc and one sialic acid residue (hereinafter referred to (GlcNAc)₂-Neu5Ac). In another embodiment, the GlcNAc and sialic acid residues are connected by virtue of one or more monosaccharides. In one embodiment, the levels of glycosylated Apo J containing N-acetylglucosamine (GlcNAc) and sialic acid residues correspond to the levels of Apo J capable of specifically binding to the Triticum vulgaris lectin.

In a preferred embodiment, the “Apo J-containing GlcNAc residues and sialic acid residues” is substantially free of other types of N-linked or O-linked carbohydrates. In one embodiment, the “Apo J-containing GlcNAc and sialic acid residues” does not contain N-linked or O-linked α-mannose residues. In another embodiment, the “Apo J-containing GlcNAc and sialic acid residues” does not contain N-linked or O-linked α-glucose residues. In yet another embodiment, the “Apo J-containing GlcNAc residues and sialic acid residues” does not contain N-linked or O-linked α-mannose residues or N-linked or O-linked α-glucose residues.

The antibody of the invention specifically recognizes an epitope which comprises a N-glycosylation site within ApoJ and said glycosylation site comprises an Asn residue selected from the group consisting of the Asn residues at positions 86, 103, 145, 291, 317, 354 or 374 with respect to the ApoJ preproprotein sequence as defined in the NCBI database entry with accession number NP_001822.3 (release of Sep. 23, 2018).

The term “epitope”, as used herein, refers to that portion of a given immunogenic substance that is the target of or is bound by an antibody or a cell-surface receptor of a host immune system that has mounted an immune response to the given immunogenic substance as determined by any method known in the art. Further, an epitope may be defined as a portion of an immunogenic substance that elicits an antibody response or induces a T-cell response in an animal, as determined by any method available in the art. See Walker J, Ed., “The Protein Protocols Handbook” (Humana Press, Inc., Totoma, N.J., US, 1996). The term “epitope” may also be used interchangeably with “antigenic determinant” or “antigenic determinant site”. The epitopes of protein antigens are divided into two categories, conformational epitopes and linear epitopes, based on their structure and interaction with the antibody.

An Asn residue refers to an asparagine amino acid within the sequence of the polypeptide. In the present embodiment, the glycosylation is linked to an Asn residue, said glycosylation is also referred as Asn-linked glycosylation, or N-linked glycosylation, and occurs when sugar residues are linked through the amide nitrogen of asparagine residues. Intracellular biosynthesis of Asn-linked oligosaccharides occurs in both the lumen of the endoplasmic reticulum and following transport of the protein to the Golgi apparatus. Asn-linked glycosylation occurs at the following tripeptide glycosylation consensus sequence: Asn-Xaa-Yaa (Asn-Xaa-Thr/Ser; NXT/S), where Xaa may be any amino acid except proline and Yaa is serine or threonine.

All Asn-linked oligosaccharides have a common pentasaccharide core (Man 3GlcNAc2) originating from a common biosynthetic intermediate. They differ in the number of branches and the presence of peripheral sugars such as fucose and sialic acid. They can be categorized according to their branched constituents, which may consist of mannose only (high mannose N-glycans); alternating GlcNAc and Gal residues terminated by various sugar sequences, and with the possibility of intrachain substitutions of bisecting Fuc and core GlcNAc (complex N-glycans); or attributes of both high mannose and complex chains (hybrid N-glycans). See, Hounsell, E. F ed., “Glycoprotein Analysis in Biomedicine,” Methods in Molecular Biology 14:298 (1993).

Alternatively, the antibody of the invention specifically recognizes or has been generated using a peptide selected from the group consisting of SEQ ID NO: 118, 119, 120, 121, 122 123 or 124, wherein the peptides are modified with N-acetylglucosamine residues at the Asn residues at position 5 in SEQ ID NO: 118, at position 5 in SEQ ID NO:119, at position 5 in SEQ ID NO:120, at position 6 in SEQ ID NO:121, at position 5 in SEQ ID NO:122, at position 5 in SEQ ID NO:123 or at position 5 in SEQ ID NO:124.

The terms “polypeptide” and “peptide” are used interchangeably herein to refer to polymers of amino acids of any length.

The term “amino acid” refers to naturally occurring and synthetic amino acids, as well as amino acid analogs and amino acid mimetics that function in a manner similar to the naturally occurring amino acids. Furthermore, the term “amino acid” includes both D- and L-amino acids (stereoisomers).

In a preferred embodiment, the antibody of the invention comprises:
a) a light chain complementarity determining region 1 (VL-CDR1) comprising an amino acid sequence set forth in any one of SEQ ID NOs: 1, 6, 11, 16, 21, 26. 31, 36, 41 or a functionally equivalent variant thereof;
b) a light chain complementarity determining region 2 (VL-CDR2) comprising an amino acid sequence set forth in any one of the amino acid sequences QAS, KAS, RAS, SAS, DAS or a functionally equivalent variant thereof;
c) a light chain complementarity determining region 3 (VL-CDR3) comprising an amino acid sequence set forth in any one of SEQ ID NOs: 2, 7, 12, 17, 22, 27, 32, 37, 42 or a functionally equivalent variant thereof,
d) a heavy chain complementarity determining region 1 (VH-CDR1) comprising an amino acid sequence set forth in any one of SEQ ID NOs: 3, 8, 13, 18, 23, 28, 33, 38, 43 or a functionally equivalent variant thereof;
e) a heavy chain complementarity determining region 2 (VH-CDR2) comprising an amino acid sequence set forth in any one of SEQ ID NOs: 4, 9, 14, 19, 24, 29, 34, 39, 44 or a functionally equivalent variant thereof or
f) a heavy chain complementarity determining region 3 (VH-CDR3) comprising an amino acid sequence set forth in any one of SEQ ID NOs: 5, 10, 15, 20, 25, 30, 35, 40, 45 or a functionally equivalent variant thereof.

The term “complementarity determining region” or “CDR”, as used herein, refers to the region within an antibody where this protein complements an antigen's shape. Thus, CDRs determine the protein's affinity (roughly, bonding strength) and specificity for specific antigens. The CDRs of the two chains of each pair are aligned by the framework regions, acquiring the function of binding a specific epitope. Consequently, both the heavy chain and the light chain are characterized by three CDRs, respectively VH-CDR1, VH-CDR2, VH-CDR3 and VL-CDR1, VL-CDR2, VL-CDR3.

As it is used herein, the term “functionally equivalent variant of a CDR sequence” refers to a sequence variant of a particular CDR sequence having substantially similar sequence identity with it and substantially maintaining its capacity to bind to its cognate antigen when being part of an antibody or antibody fragment as the ones described herein. For example, a functionally equivalent variant of a CDR sequence may be a polypeptide sequence derivative of said sequence comprising the addition, deletion or substitution of one or more amino acids.

Functionally equivalent variants of a CDR sequence according to the invention include CDR sequences having at least approximately 70%, at least 75%, at least 80%, at least 85%, at least 90%, at least 91%, at least 92%, at least 93%, at least 94%, at least 95%, at least 96%, at least 97%, at least 98% or at least 99% sequence identity with the corresponding amino acid sequences shown in one of the above reference sequences. It is also contemplated that functionally equivalent variants of a CDR sequence comprise additions consisting of at least 1 amino acid, or at least 2 amino acids, or at least 3 amino acids, or at least 4 amino acids, or at least 5 amino acids, or at least 6 amino acids, or at least 7 amino acids, or at least 8 amino acids, or at least 9 amino acids, or at least 10 amino acids or more amino acids at the N-terminus, or at the C-terminus, or both at the N- and C-terminus of the corresponding amino acid sequence shown in one of above referenced sequences. Likewise, it is also contemplated that variants comprise deletions consisting of at least 1 amino acid, or at least 2 amino acids, or at least 3 amino acids, or at least 4 amino acids, or at least 5 amino acids, or at least 6 amino acids, or at least 7 amino acids, or at least 8 amino acids, or at least 9 amino acids, or at least 10 amino acids or more amino acids at the N-terminus, or at the C-terminus, or both at the N- and C-terminus of the corresponding amino acid sequence shown in one of the above mentioned sequences.

Functionally equivalent variants of a CDR sequence according to the invention will preferably maintain at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 100%, at least 105%, at least 1 10%, at least 1 15%, at least 120%, at least 125%, at least 130%, at least 135%, at least 140%, at least 145%, at least 150%, at least 200% or more of the capacity of the corresponding amino acid sequence shown in one of SEQ ID NOs: 1 to 45 or any of light chain CDR2 sequences QAS, KAS, RAS, SAS and DAS to bind to its cognate antigen when being part of an antibody or antibody fragment as the ones of the invention. This capacity to bind to its cognate antigen may be determined as a value of affinity, avidity, specificity and/or selectivity of the antibody or antibody fragment to its cognate antigen.

In another preferred embodiment, the antibody of the invention is characterized in that:
(i) the VL-CDR1 comprises an amino acid sequence set forth in SEQ ID NO: 6, the VL-CDR2 comprises the amino acid sequence KAS and the VL-CDR3 comprises an amino acid sequence set forth in SEQ ID NO: 7,
(ii) the VL-CDR1 comprises an amino acid sequence set forth in SEQ ID NO: 1, the VL-CDR2 comprises the amino acid sequence QAS and the VL-CDR3 comprises an amino acid sequence set forth in SEQ ID NO: 2,
(iii) the VL-CDR1 comprises an amino acid sequence set forth in SEQ ID NO: 11, the VL-CDR2 comprises the amino acid sequence RAS and the VL-CDR3 comprises an amino acid sequence set forth in SEQ ID NO: 12
(iv) the VL-CDR1 comprises an amino acid sequence set forth in SEQ ID NO: 16, the VL-CDR2 comprises the amino acid sequence QAS and the VL-CDR3 comprises an amino acid sequence set forth in SEQ ID NO: 17,
(v) the VL-CDR1 comprises an amino acid sequence set forth in SEQ ID NO: 21, the VL-CDR2 comprises the amino acid sequence SAS and the VL-CDR3 comprises an amino acid sequence set forth in SEQ ID NO: 22,
(vi) the VL-CDR1 comprises an amino acid sequence set forth in SEQ ID NO: 26, the VL-CDR2 comprises the amino acid sequence DAS and the VL-CDR3 comprises an amino acid sequence set forth in SEQ ID NO: 27,
(vii) the VL-CDR1 comprises an amino acid sequence set forth in SEQ ID NO: 31, the VL-CDR2 comprises the amino acid sequence SAS and the VL-CDR3 comprises an amino acid sequence set forth in SEQ ID NO: 32,
(viii) the VL-CDR1 comprises an amino acid sequence set forth in SEQ ID NO: 36, the VL-CDR2 comprises the amino acid sequence KAS and the VL-CDR3 comprises an amino acid sequence set forth in SEQ ID NO: 37,
(ix) the VL-CDR1 comprises an amino acid sequence set forth in SEQ ID NO: 41, the VL-CDR2 comprises the amino acid sequence KAS and the VL-CDR3 comprises an amino acid sequence set forth in SEQ ID NO: 42,
(x) the VH-CDR1 comprises an amino acid sequence set forth in SEQ ID NO: 8, the VH-CDR2 comprises an amino acid sequence set forth in SEQ ID NO: 9 and the VH-CDR3 comprises an amino acid sequence set forth in SEQ ID NO: 10,
(xi) the VH-CDR1 comprises an amino acid sequence set forth in SEQ ID NO: 3, the VH-CDR2 comprises an amino acid sequence set forth in SEQ ID NO: 4 and the VH-CDR3 comprises an amino acid sequence set forth in SEQ ID NO: 5,
(xii) the VH-CDR1 comprises an amino acid sequence set forth in SEQ ID NO: 13, the VH-CDR2 comprises an amino acid sequence set forth in SEQ ID NO: 14 and the VH-CDR3 comprises an amino acid sequence set forth in SEQ ID NO: 15,
(xiii) the VH-CDR1 comprises an amino acid sequence set forth in SEQ ID NO: 18, the VH-CDR2 comprises an amino acid sequence set forth in SEQ ID NO: 19 and the VH-CDR3 comprises an amino acid sequence set forth in SEQ ID NO: 20,
(xiv) the VH-CDR1 comprises an amino acid sequence set forth in SEQ ID NO: 23, the VH-CDR2 comprises an amino acid sequence set forth in SEQ ID NO: 24 and the VH-CDR3 comprises an amino acid sequence set forth in SEQ ID NO: 25,
(xv) VH-CDR1 comprises an amino acid sequence set forth in SEQ ID NO:

28, the VH-CDR2 comprises an amino acid sequence set forth in SEQ ID NO: 29 and the VH-CDR3 comprises an amino acid sequence set forth in SEQ ID NO: 30,
(xvi) the VH-CDR1 comprises an amino acid sequence set forth in SEQ ID NO: 33, the VH-CDR2 comprises an amino acid sequence set forth in SEQ ID NO: 34 and the VH-CDR3 comprises an amino acid sequence set forth in SEQ ID NO: 35,
(xvii) the VH-CDR1 comprises an amino acid sequence set forth in SEQ ID NO: 38, the VH-CDR2 comprises an amino acid sequence set forth in SEQ ID NO: 39 and the VH-CDR3 comprises an amino acid sequence set forth in SEQ ID NO: 40 or
(xviii) the VH-CDR1 comprises an amino acid sequence set forth in SEQ ID NO: 43, the VH-CDR2 comprises an amino acid sequence set forth in SEQ ID NO: 44 and the VH-CDR3 comprises an amino acid sequence set forth in SEQ ID NO: 45.

In another embodiment, the above mentioned antibody is characterized in that:
(i) the VL-CDR1 comprises an amino acid sequence set forth in SEQ ID NO: 6, the VL-CDR2 comprises the amino acid sequence KAS, the VL-CDR3 comprises an amino acid sequence set forth in SEQ ID NO: 7, the VH-CDR1 comprises an amino acid sequence set forth in SEQ ID NO: 8, the VH-CDR2 comprises an amino acid sequence set forth in SEQ ID NO: 9 and the VH-CDR3 comprises an amino acid sequence set forth in SEQ ID NO: 10,
(ii) the VL-CDR1 comprises an amino acid sequence set forth in SEQ ID NO: 1, the VL-CDR2 comprises the amino acid sequence QAS, the VL-CDR3 comprises an amino acid sequence set forth in SEQ ID NO: 2, the VH-CDR1 comprises an amino acid sequence set forth in SEQ ID NO: 3, the VH-CDR2 comprises an amino acid sequence set forth in SEQ ID NO: 4 and the VH-CDR3 comprises an amino acid sequence set forth in SEQ ID NO: 5,
(iii) the VL-CDR1 comprises an amino acid sequence set forth in SEQ ID NO: 11, the VL-CDR2 comprises the amino acid sequence RAS wherein the VL-CDR3 comprises an amino acid sequence set forth in SEQ ID NO: 12, the VH-CDR1 comprises an amino acid sequence set forth in SEQ ID NO: 13, the VH-CDR2 comprises an amino acid sequence set forth in SEQ ID NO: 14 and the VH-CDR3 comprises an amino acid sequence set forth in SEQ ID NO: 15,
(iv) the VL-CDR1 comprises an amino acid sequence set forth in SEQ ID NO: 16, the VL-CDR2 comprises the amino acid sequence QAS, the VL-CDR3 comprises an amino acid sequence set forth in SEQ ID NO: 17, the VH-CDR1 comprises an amino acid sequence set forth in SEQ ID NO: 18, the VH-CDR2 comprises an amino acid sequence set forth in SEQ ID NO: 19 and the VH-CDR3 comprises an amino acid sequence set forth in SEQ ID NO: 20,
(v) the VL-CDR1 comprises an amino acid sequence set forth in SEQ ID NO: 21, the VL-CDR2 comprises the amino acid sequence SAS, the VL-CDR3 comprises an amino acid sequence set forth in SEQ ID NO: 22, the VH-CDR1 comprises an amino acid sequence set forth in SEQ ID NO: 23, the VH-CDR2 comprises an amino acid sequence set forth in SEQ ID NO: 24 and the VH-CDR3 comprises an amino acid sequence set forth in SEQ ID NO: 25,
(vi) the VL-CDR1 comprises an amino acid sequence set forth in SEQ ID NO: 26, the VL-CDR2 comprises the amino acid sequence DAS, the VL-CDR3 comprises an amino acid sequence set forth in SEQ ID NO: 27, the VH-CDR1 comprises an amino acid sequence set forth in SEQ ID NO: 28, the VH-CDR2 comprises an amino acid sequence set forth in SEQ ID NO: 29 and the VH-CDR3 comprises an amino acid sequence set forth in SEQ ID NO: 30,
(vii) the VL-CDR1 comprises an amino acid sequence set forth in SEQ ID NO: 31, the VL-CDR2 comprises the amino acid sequence SAS the VL-CDR3 comprises an amino acid sequence set forth in SEQ ID NO: 32, the VH-CDR1 comprises an amino acid sequence set forth in SEQ ID NO: 33, the VH-CDR2 comprises an amino acid sequence set forth in SEQ ID NO: 34 and the VH-CDR3 comprises an amino acid sequence set forth in SEQ ID NO: 35,
(viii) the VL-CDR1 comprises an amino acid sequence set forth in SEQ ID NO: 36, the VL-CDR2 comprises the amino acid sequence KAS, the VL-CDR3 comprises an amino acid sequence set forth in SEQ ID NO: 37, the VH-CDR1 comprises an amino acid sequence set forth in SEQ ID NO: 38, the VH-CDR2 comprises an amino acid sequence set forth in SEQ ID NO: 39 and the VH-CDR3 comprises an amino acid sequence set forth in SEQ ID NO: 40,
(ix) the VL-CDR1 comprises an amino acid sequence set forth in SEQ ID NO: 41, the VL-CDR2 comprises the amino acid sequence KAS, the VL-CDR3 comprises an amino acid sequence set forth in SEQ ID NO: 42, the VH-CDR1 comprises an amino acid sequence set forth in SEQ ID NO: 43, the VH-CDR2 comprises an amino acid sequence set forth in SEQ ID NO: 44 and the VH-CDR3 comprises an amino acid sequence set forth in SEQ ID NO: 45,

In another embodiment, the antibody of the invention is characterized in that:
(i) a light chain framework 1 (VL-FR1) region amino acid sequence at least 90% identical to the amino acid sequence set forth in any one of SEQ ID NOs: 46, 54, 62, 70, 78, 86, 94, 102 or 110,
(ii) a light chain framework 2 (VL-FR2) region amino acid sequence at least 90% identical to the amino acid sequence set forth in any one of SEQ ID NOs: 47, 55, 63, 71, 79, 87, 95, 103 or 111,
(iii) a light chain framework 3 (VL-FR3) region amino acid sequence at least 90% identical to the amino acid sequence set forth in any one of SEQ ID NOs: 48, 56, 64, 72, 80, 88, 96, 104 or 112 and
(iv) a light chain framework 4 (VL-FR4) region amino acid sequence at least 90% identical to the amino acid sequence set forth in any one of SEQ ID NOs: 49, 57, 65, 73, 81, 89, 97, 105 or 113.

In another embodiment, the antibody of the invention further comprises one or more of:

(i) a heavy chain framework 1 (VH-FR1) region amino acid sequence at least 90% identical to the amino acid sequence set forth in any one of SEQ ID NOs: 50, 58, 66, 74, 82, 90, 98, 106 or 114,
(ii) a heavy chain framework 2 (VH-FR2) region amino acid sequence at least 90% identical to the amino acid sequence set forth in any one of SEQ ID NOs: 51, 59, 67, 75, 83, 91, 99, 107 or 115,
(iii) a heavy chain framework 3 (VH-FR3) region amino acid sequence at least 90% identical to the amino acid sequence set forth in any one of SEQ ID NOs: 52, 60, 68, 76, 84, 92, 100, 108 or 116 and
(iv) a heavy chain framework 4 (VH-FR4) region amino acid sequence at least 90% identical to the amino acid sequence set forth in any one of SEQ ID NOs: 53, 61, 69, 77, 85, 93, 101, 109 or 117.

In another embodiment, the antibody of the invention is the Ag1G-11, Ag2G-17, Ag3G-4, Ag4g-6, Ag5G-17, Ag6G-1, Ag6G-11, Ag7G-17 or the Ag7g-19 antibody wherein the respective VL-FR1, VL-CDR1, VL-FR2, VL-CDR2, VL-FR3, VL-CDR3, VL-FR4, VH-FR1, VH-CDR1, VH-FR2, VH-CDR2, VH-FR3, VH-CDR3 and VH-FR4 regions of each antibody comprise the amino acid sequences set forth in Table 1.

TABLE 1
Correspondence between the different CDR and framework regions in each antibody and their SEQ ID NO:
as referred in the text.

| | LIGHT CHAIN | | HEAVY CHAIN | |
|---|---|---|---|---|
| | REGION | SEQ ID and/or aa SEQUENCE | REGION | SEQ ID and/or aa SEQUENCE |
| Ag1G-11 | FWR1 | DPVLTQTPSPVSAAVGGTVTINCQAS | FWR1 | QSLEESGGRLVTPGGSLTLTCTVS |
| | | (SEQ ID NO: 46) | | (SEQ ID NO: 50) |
| | CDR1 | QSVYNNNE | CDR1 | GFSLSSYA |
| | | (SEQ ID NO: 1) | | (SEQ ID NO: 3) |
| | FWR2 | LSWYQQKPGQPPKPLIY | FWR2 | MSWVRQAPGKGLEWIGI |
| | | (SEQ ID NO: 47) | | (SEQ ID NO: 51) |
| | CDR2 | QAS | CDR2 | IGVNGDT |
| | | | | (SEQ ID NO: 4) |
| | FWR3 | KLASGVPSRFKGSGSGTQFTLTISDLECDDAATYYC | FWR3 | YYASWAKGRFTISKTSTTVGLKITSPTTEDTATYFC |
| | | (SEQ ID NO: 48) | | (SEQ ID NO: 52) |
| | CDR3 | QGIYLGSDWYDV | CDR3 | ARVRYPYYDTDAFDP |
| | | (SEQ ID NO: 2) | | (SEQ ID NO: 5) |
| | FWR4 | FGGGTEVVVKRTVAAPSVFIFPPSDEQLKSGTASVVC | FWR4 | WGPGTLVTISSASTKGPSVFPLAPSSKSTSGGTAALG |
| | | LLNNFYPREAKVQWKVDNALQSGNSQESVTEQDSKDS | | CLVKDYFPEPVTVSWNSGALTSGVHTFPAVLQSSGLY |
| | | TYSLSSTLTLSRADYEKHKVYACEVTHQGLSSPVTKS | | SLSSVVTVPSSSLGTQTYICNVNHKPSNTKVDKKVEP |
| | | FNRGEC | | KSCDKTSGSHHHHHH |
| | | (SEQ ID NO: 49) | | (SEQ ID NO: 53) |
| Ag2G-17 | FWR1 | MTQTPASASEPVGGTVTIKCQAS | FWR1 | QEQLKESGGRLVTPGGSLTLTCTVA |
| | | (SEQ ID NO: 54) | | (SEQ ID NO: 58) |
| | CDR1 | QSIGNL | CDR1 | GFSLSRYP |
| | | (SEQ ID NO: 6) | | (SEQ ID NO: 8) |
| | FWR2 | LAWYQQKPGQPPKFLIY | FWR2 | MN*VRQAPGKGLEWIGV |
| | | (SEQ ID NO: 55) | | (SEQ ID NO: 59) |
| | CDR2 | KAS | CDR2 | ISSGGWLT |
| | | | | (SEQ ID NO: 9) |
| | FWR3 | TLASGVSSQFKGSGSGTEFTLTISDLECADAATYY | FWR3 | FYANWAKGRFTISKTSTTVDLKITSPTTEDTTTYFC |
| | | C | | (SEQ ID NO: 60) |
| | | (SEQ ID NO: 56) | | |
| | CDR3 | QSYYAIASYGVA | CDR3 | ARFGRYGNTDYYYFDL |
| | | (SEQ ID NO: 7) | | (SEQ ID NO: 10) |
| | FWR4 | FGAGTEVVVKRTVAAPSVFIFPPSDEQLKSGTASV | FWR4 | WGQGTLVTVSSASTKGPSVFPLAPSSKSTSVTVSWNS |
| | | VCLLNNFYPREAKVQWKVDNALQSGNSQESVTEQD | | GALTSGVHTFPAVLQSSGLYSLSSVVTVPSSSLGTQT |
| | | SKDSTYSLSSTLTLSKADYEKHKVYACEVTHQGLS | | YICNVNHKPSNTKVDKKVEPKSCDKTSGSHHHHHH |
| | | SPVTKSFNRGEC | | (SEQ ID NO: 61) |
| | | (SEQ ID NO: 57) | | |
| Ag3G-4 | FWR1 | DVVMTQTPASVEAAVGGTVTIKCQAS | FWR1 | QEQLEESGGRLVTPGTPLTLTCAVS |
| | | (SEQ ID NO: 62) | | (SEQ ID NO: 66) |
| | CDR1 | QSISTY | CDR1 | GFSLSSYG |
| | | (SEQ ID NO: 11) | | (SEQ ID NO: 13) |
| | FWR2 | LSWHQQKPGQPPKLLIY | FWR2 | VSWVRQAPGKGLEYIGY |
| | | (SEQ ID NO: 63) | | (SEQ ID NO: 67) |
| | CDR2 | RAS | CDR2 | IDVSGSA |
| | | | | (SEQ ID NO: 14) |
| | FWR3 | TLESGVPSRFKGSGSGTEFALTISDLECADAATYYC | FWR3 | YYASWARGRFTISRTSTTVDLKMTSLTTEDTATYFC |
| | | (SEQ ID NO: 64) | | (SEQ ID NO: 68) |
| | CDR3 | QTAHDSSGRGTWGVI | CDR3 | ARGSPGYDANDL |
| | | (SEQ ID NO: 12) | | (SEQ ID NO: 15) |
| | FWR4 | FGGGTEVVVKRTVAAPSVFIFPPSDEQLKSGTASVV | FWR4 | WGQGTLVTISSASTKGPSVFPLAPSSKSTSGGTAALGCL |
| | | CLLNNFYPREAKVQWKVDNALQSGNSQESVTEQDSK | | VKDYFPEPVTVSWNSGALTSGVHTFPAVLQSSGLYSLSS |
| | | DSTYSLSSTLTLSKADYEKHKVYACEVTHQGLSSPV | | VVTVPSSSLGTQTYICNVNHKPSNTKVDKKVEPKSCDKT |
| | | TKSFNRGEC | | SGSHHHHHHAAATE |
| | | (SEQ ID NO: 65) | | (SEQ ID NO: 69) |
| Ag4G-6 | FWR1 | MTQTPASVEVAVGGTVTIKCQAS | FWR1 | QQLEESGGRLVTPGTPLTLTCTAS |
| | | (SEQ ID NO: 70) | | (SEQ ID NO: 74) |
| | CDR1 | QSINGY | CDR1 | GMDLSKYW |
| | | (SEQ ID NO: 16) | | (SEQ ID NO: 18) |
| | FWR2 | LAWYQQKPGQPPKLLIY | FWR2 | MTWVRQAPGKGLEYIGI |
| | | (SEQ ID NO: 71) | | (SEQ ID NO: 75) |
| | CDR2 | QAS | CDR2 | IETGGSA |
| | | | | (SEQ ID NO: 19) |
| | FWR3 | TLASGVSSRFQGSGSGTEYTLTISGVQCDDAATY | FWR3 | YYASWAKGRFTISRTSTTVDLKMISPTTEDTATYFC |
| | | YC | | (SEQ ID NO: 76) |
| | | (SEQ ID NO: 72) | | |
| | CDR3 | QGDYYGWIRT | CDR3 | GRWGDI |
| | | (SEQ ID NO: 17) | | (SEQ ID NO: 20) |
| | FWR4 | FGGGTEVVVKRTVAAPSVFIFPPSDEQLKSGTAS | FWR4 | WGPGTLVTVSSASTKGPSVFPLAPSSKSTSGGTAAL |
| | | VVCLLNNFYPREAKVQWKVDNALQSGNSQESVTE | | GCLVKDYFPEPVTVSWNSGALTSGVHTFPAVLQSSG |
| | | QDSKDSTYSLSSTLTLSKADYEKHKVYACEVTHQ | | LYSLSSVVTVPSSSLGTQTYICNVNHKPSNTKVDKK |
| | | GLSSPVTKSFNRGEC | | VEPKSCDKTSGSHHHHHH |
| | | (SEQ ID NO: 73) | | (SEQ ID NO: 77) |
| Ag5G-17 | FWR1 | DVVMTQTPSSKSVPVGDTVTINCQAS | FWR1 | QEQLVESGGRLVTPGTPLILTCTAS |
| | | (SEQ ID NO: 78) | | (SEQ ID NO: 82) |
| | CDR1 | ESVYVNNF | CDR1 | GFSLSRHT |
| | | (SEQ ID NO: 21) | | (SEQ ID NO: 23) |
| | FWR2 | LSWYRQKPGQPPKRLIY | FWR2 | MSWVRQAPGKGLEWIGY |
| | | (SEQ ID NO: 79) | | (SEQ ID NO: 83) |
| | CDR2 | SAS | CDR2 | ITYGGSA |
| | | | | (SEQ ID NO: 24) |
| | FWR3 | TLASGVPSRFSGSGSGTQFTLTISDVVCDDAATYYC | FWR3 | YSANWAKGRFTISRTSTTVDLKMNSLTTEDTATYFC |
| | | (SEQ ID NO: 80) | | (SEQ ID NO: 84) |
| | CDR3 | AGYHEFNTDGNA | CDR3 | GRVGAYGAYYDL |
| | | (SEQ ID NO: 22) | | (SEQ ID NO: 25) |
| | FWR4 | FGGGTEVVVKRTVAAPSVFIFPPSDEQLKSGTASVVC | FWR4 | WGQGTLVTVSSASTKGPSVFPLAPSSKSTSGGTAALGCL |
| | | LLNNFYPREAKVQWKVDNALQSGNSQESVTEQDSKDS | | VKDYFPEPVTVSWNSGALTSGVHTFPAVLQSSGLYSLSS |
| | | TYSLSSTLTLSKADYEKHKVYACEVTHQGLSSPVTKS | | VVTVPSSSLGTQTYICNVNHKPSNTKVDKKVEPKSCDKT |
| | | FNRGEC | | SGSHHHHHHAPLR |
| | | (SEQ ID NO: 81) | | (SEQ ID NO: 85) |
| Ag6G-1 | FWR1 | DPMLTQTPSSVSAAVGGTVTANCQSS | FWR1 | QSVEESGGRLVTPGTPLTFSCTAS |
| | | (SEQ ID NO: 86) | | (SEQ ID NO: 90) |
| | CDR1 | QSVRGNND | CDR1 | GFSLNSYY |
| | | (SEQ ID NO: 26) | | (SEQ ID NO: 28) |
| | FWR2 | LAWYQQKPGQPPKLLIY | FWR2 | MSWVRQAPGKGLEWIGL |
| | | (SEQ ID NO: 87) | | (SEQ ID NO: 91) |
| | CDR2 | DAS | CDR2 | VSTDGSA |
| | | | | (SEQ ID NO: 29) |
| | FWR3 | KLASGVPSRFKGSGSGTDFTLTISDLECADAATY | FWR3 | YYASWAKGRFTISRTSTTVDLKLTSLTTEDAATYF |
| | | YC | | C |
| | | (SEQ ID NO: 88) | | (SEQ ID NO: 92) |
| | CDR3 | QQGHRVEDVANA | CDR3 | ARDRGSDSYIDQLDL |
| | | (SEQ ID NO: 27) | | (SEQ ID NO: 30) |
| | FWR4 | FGGGTEVVVKRTVAAPSVFIFPPSDEQLKSGTAS | FWR4 | WGQGTLVTISSASTKGPSVFPLAPSSKSTSGGTAA |
| | | VVCLLNNFYPREAKVQWKVDNALQSGNSQESVTE | | LGCLVKDYFPEPVTVSWNSGALTSGVHTFPAVLQS |
| | | QDSKDSTYSLSSTLTLSKADYEKHKVYACEVTHQ | | SGLYSLSSVVTVPSSSLGTQTYICNVNHKPSNTKV |
| | | GLSSPVTKSFNRGEC | | DKKVEPKSCDKTSGSHHHHHHAAAT |
| | | (SEQ ID NO: 89) | | (SEQ ID NO: 93) |
| Ag6G-11 | FWR1 | DPMLTQTPSSVSAPVGGTVTIKCQAS | FWR1 | QEQLEESGGRLVTPGTPLTLTCTVS |
| | | (SEQ ID NO: 94) | | (SEQ ID NO: 98) |
| | CDR1 | ESISSR | CDR1 | GFSLSTYN |
| | | (SEQ ID NO: 31) | | (SEQ ID NO: 33) |
| | FWR2 | LAWYQQKPGQRPKLLTY | FWR2 | MGWVRQAPGKGLEYIGI |
| | | (SEQ ID NO: 95) | | (SEQ ID NO: 99) |
| | CDR2 | SAS | CDR2 | IYGSGSV |
| | | | | (SEQ ID NO: 34) |
| | FWR3 | TLASGVSSRFKGSGSETQFTLTISDVQCDDAATY | FWR3 | QYATWAKGRFTISKTSTTVDLKITSLTTEDTATY |
| | | YC | | FC |
| | | (SEQ ID NO: 96) | | (SEQ ID NO: 100) |
| | CDR3 | LGTYSAIRT | CDR3 | ARMGSGWGFNI |
| | | (SEQ ID NO: 32) | | (SEQ ID NO: 35) |
| | FWR4 | FGGGTEVVVKRTVGRHLSSSSRHLMSS | FWR4 | WGPGTLVPSPQLAPRAHRSSRWHPPPRAPLGAQR |
| | | (SEQ ID NO: 97) | | PWAAWSRTTSPNP |
| | | | | (SEQ ID NO: 101) |
| Ag7G-17 | FWR1 | YVMMTQTPASVSEPVGGTVTIKCQAS | FWR1 | MAQSLEESGGRLVTPGTPLTLTCTAS |
| | | (SEQ ID NO: 102) | | (SEQ ID NO: 106) |
| | CDR1 | QSIDSY | CDR1 | GFSLNTYYW |
| | | (SEQ ID NO: 36) | | (SEQ ID NO: 38) |
| | FWR2 | LAWYQQKPGQPPKLLTY | FWR2 | VSWVRQAPGKGLEWIGH |
| | | (SEQ ID NO: 103) | | (SEQ ID NO: 107) |
| | CDR2 | KAS | CDR2 | INPDGTA |
| | | | | (SEQ ID NO: 39) |
| | FWR3 | TLASGVPSRFSGSGSGTQFTLTISDVECDDAATY | FWR3 | YYATWAKGRFTISRTSTTVDLKITSPTTEDTATYF |
| | | YC | | C |
| | | (SEQ ID NO: 104) | | (SEQ ID NO: 108) |
| | CDR3 | QCSHYGSNWLGP | CDR3 | ARLGSTGDNFNI |
| | | (SEQ ID NO: 37) | | (SEQ ID NO: 40) |
| | FWR4 | FGGGTEVVVKRTVAAPSVFIFPPSDEQLKSGTAS | FWR4 | WGPGTLVTISSASTKGPSVFPLAPSSKSTSGGTAA |
| | | VVCLLNNFYPREAKVQWKVDNALQSGNSQESVTE | | LGCLVKDYFPEPVTVSWNSGALTSGVHTFPAVLQS |
| | | QDSKDSTYSLSNTLTLSKADYEKHKVYACEVTHQ | | SGLYSLSSVVTVPSSSLGTQTYICNVNHKPSNTKV |
| | | GLSSPVTESFNRGEC | | DKKVEPKSCDKTSGSHHHHHHAAATER |
| | | (SEQ ID NO: 105) | | (SEQ ID NO: 109) |
| Ag7G-19 | FWR1 | YVMMTQTPSSTSAAVGGTVTINCQSS | FWR1 | QSVEESGGRLVTPGTPLTLTCTVS |
| | | (SEQ ID NO: 110) | | (SEQ ID NO: 114) |
| | CDR1 | QSVYSNSF | CDR1 | GIDLSSNA |
| | | (SEQ ID NO: 41) | | (SEQ ID NO: 43) |
| | FWR2 | LSWYQQKPGQLPKLLIY | FWR2 | MSWVRQAPGGGLEWIGT |
| | | (SEQ ID NO: 111) | | (SEQ ID NO: 115) |
| | CDR2 | KAS | CDR2 | INTNGKT |
| | | | | (SEQ ID NO: 44) |
| | FWR3 | TLASGVPSRFKGSGSGTQFTLTISELQCDDAATY | FWR3 | YDASWMNGRFTISKTSSTTVDLKMTSLTTEDTAT |
| | | YC | | YFC |
| | | (SEQ ID NO: 112) | | (SEQ ID NO: 116) |
| | CDR3 | QGGYVDWMRA | CDR3 | ARGRWTGNTGWYIDL |
| | | (SEQ ID NO: 42) | | (SEQ ID NO: 45) |
| | FWR4 | FGGGTEVVVKRTVAAPSVFIFPPSDEQLKSGTAS | FWR4 | WGPGTLVTVSSASTKGPSVFPLAPSSKSTSGGTA |
| | | VVCLLNNFYPREAKVQWKVDNALQSGNSQESVTE | | ALGCLVKDYFPEPVTVSWNSGALTSGVHTFPAVL |
| | | QDSKDSTYSLSSTLTLSKADYEKHKVYACEVTHQ | | QSSGLYSLSSVVTVPSSSLGTQTYICNVNHKPSN |
| | | GLSSPVTKSFNRGEC | | TKVDKKVEPKSCDKTSGSHHHHHHAAATER |
| | | (SEQ ID NO: 113) | | (SEQ ID NO: 117) |

In another preferred embodiment, the antibody according to the invention comprises
i) a light chain defined by the amino acid sequence set forth in any one of SEQ ID NOs: 125, 126, 127, 128, 129, 130, 131, 132 or 133, and/or
ii) a heavy chain defined by the amino acid sequence set forth in any of SEQ ID Nos: 134, 135, 136, 137, 138, 139, 140, 141 or 142.

In a further embodiment, the antibody of any of the invention comprises at least one framework region derived from a human antibody framework region, which is humanised or which is super-humanised.

By “humanised” is meant an antibody derived from a non-human antibody, typically a murine antibody, that retains the antigen-binding properties of the parent antibody, but which is less immunogenic in humans. This may be achieved by various methods, including (a) grafting the entire non-human variable domains onto human constant regions to generate chimeric antibodies; (b) grafting only the non-human complementarity determining regions (CDRs) into human framework and constant regions with or without retention of critical framework residues; and (c) transplanting the entire non-human variable domains, but “cloaking” them with a human-like section by replacement of surface residues. Methods for humanizing non-human antibodies have been described in the art. Preferably, a humanised antibody has one or more amino acid residues introduced into it from a source which is non-human. These non-human amino acid residues are often referred to as “import” residues, which are typically taken from an “import” variable domain.

Humanisation can be essentially performed following the method of Winter and co-workers (Jones et al., Nature, 321:522-525 (1986); Reichmann et al., Nature, 332:323-327 (1988); Verhoeyen et al., Science, 239: 1534-1536 (1988)), by substituting hypervariable region sequences for the corresponding sequences of a human antibody. In practice, humanised antibodies are typically human antibodies in which some hypervariable region residues and possibly some framework region (FR) residues are substituted by residues from analogous sites in rodent antibodies. The choice of human variable domains, both light and heavy, to be used in making the humanised antibodies is very important to reduce immunogenicity retaining the specificity and affinity for the antigen. According to the so called “best-fit” method, the sequence of the variable domain of a rodent antibody is screened against the entire library of known human variable-domain sequences. The human sequence which is closest to that of the rodent is then accepted as the human framework region (FR) for the humanised antibody (Suns et al., J. Immunol, 151:2296 (1993); Chothia et al., J. Mol. Biol, 196:901 (1987)). Another method uses a particular framework region derived from the consensus sequence of all human antibodies of a particular subgroup of light or heavy chains. The same framework may be used for several different humanised antibodies (Carter et al., Proc. Natl. Acad. Sci. USA, 89:4285 (1992); Presta et al., J. Immunol, 151:2623 (1993)).

It is further important that antibodies are humanised with retention of high affinity for the antigen and other favorable biological properties. To achieve this goal, humanised antibodies are prepared by a process of analysis of the parental sequences and various conceptual humanised products using three-dimensional models of the parental and humanised sequences. A further step in this approach, to make an antibody more similar to humans, is to prepare the so called primatised antibodies, i.e. a recombinant antibody which has been engineered to contain the variable heavy and light domains of a monkey (or other primate) antibody, in particular, a cynomolgus monkey antibody, and which contains human constant domain sequences, preferably the human immunoglobulin gamma 1 or gamma 4 constant domain (or PE variant).

By “human antibody” is meant an antibody containing entirely human light and heavy chains as well as constant regions, produced by any of the known standard methods.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region PH gene in chimeric and germ-line mutant mice results in the complete inhibition of endogenous antibody production. Transfer of the human germ-line immunoglobulin gene array in such germ line mutant mice will result in the production of human antibodies after immunization. See, e.g., Jakobovits et al, Proc. Mad. Acad. Sci. USA, 90:255 1 (1993); Jakobovits et al, Nature, 362:255-258 (1993), Lonberg, 2005, Nature Biotech. 23: 1117-25.

Human antibodies may also be generated by in vitro activated B cells or SCID mice with its immune system reconstituted with human cells.

Once a human antibody is obtained, its coding DNA sequences can be isolated, cloned and introduced into an appropriate expression system, i.e., a cell line, preferably from a mammal, which subsequently express and liberate it into a culture media from which the antibody can be isolated.

In another preferred embodiment, the antibody of the invention is a Fab, a F(ab)₂, a single-domain antibody, a single chain variable fragment (scFv), or a nanobody.

Fab, F(ab)2, single-domain antibodies, single chain variable fragments (scFv), and nanobodies are considered epitope-binding antibody fragments.

An antibody fragment is a fragment of an antibody such as, for example, Fab, F(ab′)₂, Fab′ and scFv. Various techniques have been developed for the production of antibody fragments. Traditionally, these fragments were derived via proteolytic digestion of intact antibodies but more recently these fragments can be produced directly by recombinant host cells. In other embodiments, the antibody of choice is a single chain Fv (scFv) fragment which additionally may be monospecific or bispecific.

Papain digestion of antibodies produces two identical antigen-binding fragments, called “Fab” fragments, each with a single antigen-binding site, and a residual “Fc” fragment, which name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind the antigen, although with lower affinity than the entire binding site.

The Fab fragment also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear at least one free thiol group. F(ab′)Z antibody fragments originally were produced as pairs of Fab′ fragments which have hinge cysteines between them. Other chemical couplings of antibody fragments are also known.

The term “single domain antibodies” makes reference to antibodies whose complementary determining regions are part of a single domain polypeptide. Examples include, but are not limited to, heavy chain antibodies (nanobodies), antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine.

“Single-chain Fv” or “scFv” antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, N.Y., pp. 269-315 (1994).

More preferably, although the two domains of the Fv fragment, VL and VH, are naturally encoded by separate genes, or polynucleotides that encode such gene sequences (e.g., their encoding cDNA) can be joined, using recombinant methods, by a flexible linker that enables them to be made as a single protein chain in which the VL and VH regions associate to form monovalent epitope-binding molecules (known as single-chain Fv (scFv). Alternatively, by employing a flexible linker that is too short (e.g., less than about 9 residues) to enable the VL and VH domains of a single polypeptide chain to associate together, one can form a bispecific antibody, diabody, or similar molecule (in which two such polypeptide chains associate together to form a bivalent epitope-binding molecule. Examples of epitope-binding antibody fragments encompassed within the present invention include (i) a Fab′ or Fab fragment, a monovalent fragment consisting of the VL, VH, CL and CH1 domains, or a monovalent antibody as described in WO2007059782; (ii) F(ab′)2 fragments, bivalent fragments comprising two Fab fragments linked by a disulfide bridge at the hinge domain; (iii) an Fd fragment consisting essentially of the VH and CH1 domains; (iv) a Fv fragment consisting essentially of a VL and VH domains, (v) a dAb fragment, which consists essentially of a VH domain and also called domain antibodies; (vi) camelid or nanobodies and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH domains pair to form monovalent molecules (known as single chain antibodies or single chain Fv (scFv).

The term “nanobodies” designates small sized entities (15 kDa) formed solely by the antigen binding region of the heavy chain (VH fragment) of immunoglobulins. Said nanobodies are mainly produced after immunizing animals of the Camelidae family, such as camels, llamas and dromedaries, mainly llamas; and also of the shark family, which have the particularity of having antibodies which naturally lack the light chain and recognize the antigen by the heavy chain variable domain. Nevertheless, the nanobodies derived from these sources require a humanization process for their therapeutic application. Another potential source for obtaining nanobodies is from antibodies derived from different human samples by separating the VH and VL domains of the variable region. Nanobodies present advantages such as a production cost reduction with respect to whole antibodies, stability and the reduction of immunogenicity.

The term “diabodies” refers to small antibody fragments with two antigen-binding sites, those fragments comprising a heavy-chain variable domain (V_{H}) connected to a light chain variable domain (V_{L}) in the same polypeptide chain (V_{H}—V_{L}). By using a linker that is too short to allow pairing between the two domains on the same chain, the domains are forced to pair with the complementary domains of another chain and create two antigen-binding sites.

Functional fragments of antibodies which bind to glycosylated ApoJ included within the present invention retain at least one binding function and/or modulation function of the full-length antibody from which they are derived. Preferred functional fragments retain an antigen-binding function of a corresponding full-length antibody (e.g., the ability to bind a mammalian CCR9).

The invention may also include bispecific antibodies. Bispecific antibodies are antibodies that have binding specificities for at least two different epitopes. Exemplary bispecific antibodies may bind to two different epitopes of the glycosylated ApoJ. Bispecific antibodies can be prepared as full-length antibodies or antibody fragments (e.g. F(ab)₂ bispecific antibodies, minibodies, diabodies). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CHI) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Techniques for generating bispecific antibodies from antibody fragments have also been described in the literature. For example, bispecific antibodies can be prepared using chemical linkage.

The fragments with the capacity to bind to glycosylated ApoJ can also be obtained by conventional methods known by persons having ordinary skill in the art. Said methods can involve isolating DNA that encodes the polypeptide chains (or a fragment thereof) of a monoclonal antibody of interest and manipulating DNA by means of recombinant DNA technology. DNA can be used to generate another DNA of interest, or an altered DNA, (for example by means of mutagenesis) for adding, removing or substituting one or more amino acids, for example, the DNA that encodes the polypeptide chains of an antibody (e.g., the heavy or light chains, the variable region or the whole antibody) can be isolated from murine B cells from immunized mice with glycosylated ApoJ. The DNA can be isolated and amplified by conventional methods, for example by means of PCR.

The single-chain antibodies can be obtained by conventional methods by binding the variable region of the heavy and light chains (Fv region) by means of an amino acid bridge. The scFvs can be prepared by fusing the DNA encoding a linker peptide between the DNAs encoding the polypeptides of the variable regions (VL and VH). The production of scFvs is described in a number of documents, for example, in U.S. Pat. No. 4,946,778, Bird (Science 242: 423, 1988), Huston et al. (Proc. Natl. Acad Sci USA 85: 5879, 1988) and Ward et al. (Nature 334: 544, 1989).

In another embodiment, the antibody comprises a VL domain and a VH domain. The term “VH domain” refers to the amino terminal variable domain of an immunoglobulin heavy chain, and the term “VL domain” refers to the amino terminal variable domain of an immunoglobulin light chain. The VL domain described herein may be linked to a constant domain to form a light chain, e.g., a full-length light chain. The VH domain described herein may be linked to a constant domain to form a heavy chain, e.g., a full-length heavy chain

In a further embodiment, the antibody of the invention is coupled to a detectable label. In another preferred embodiment, said label can be detected by means of a change in at least one of its physical, chemical, electrical or magnetic properties.

In the context of the present invention, the term “detectable label” or “labelling agent”, as used herein, refers to a molecular label which allows the detection, localization and/or identification of the molecule to which it is attached, using suitable procedures and equipment for detection, for example by spectroscopic, photochemical, biochemical, immunochemical or chemical means. Labelling agents that are suitable for labelling the antibodies include radionuclides, enzymes, fluorophores, chemiluminescent reagents, enzyme substrates or cofactors, enzyme inhibitors, particles, magnetic particles, dyes and derivatives, and the like.

The compounds radioactively labeled by means of radioactive isotopes, also called radioisotopes or radionuclides, may include, without limitation, ³H, ¹⁴C, ¹⁵N, ³⁵S, ⁹⁰Y, ⁹⁹Tc, ^{m}In, ¹²⁵I, ¹³¹I, ¹³³Xe, ¹¹¹Lu, ²¹¹At and ²¹³B. Radioisotope labelling is performed typically by using chelating ligands that are capable of complexing metal ions such as DOTA, DOTP, DOTMA, DTPA and TETA. Methods for conjugating radioisotopes to proteins are well known in the prior art.

In another particular embodiment, the antibody of the invention is labelled with a fluorescent group. The fluorescent group can be attached to the side chains of the amino acids directly or through a linking group. Methods for conjugating polypeptides fluorescent reagents are well known in the prior art.

Suitable reagents for labelling polypeptides, such as antibodies, with fluorescent groups include chemical groups which show ability to react with the various groups listed in the side chains of the proteins, including amino groups and thiol groups. Thus, chemical groups that can be used to modify the antibodies according to the present invention include, without limitation, maleimide, haloacetyl, iodoacetamide succinimidyl ester (e.g. NHS, N-hydroxysuccinimide), isothiocyanate, sulfonyl chloride, 2,6-dichlorotriazinyl, pentafluorophenyl ester, phosphoramidite and the like. An example of suitable reactive functional group is N-hydroxysuccinimide ester (NHS) of a detectable group modified with a carboxyl group. Typically, the carboxyl group modifying the fluorescent compound is activated by the contacting of said compound with a carbodiimide reagent (for example, dicyclohexylcarbodiimide, diisopropylcarbodiimide, uranium or a reagent such as TSTU (0-(N-Succinimidyl)-N,N,N′,N′-tetramethyluronium tetrafluoroborate), H BTU ((0-benzotriazol-1-yl)-N, N, N′,N′-tetramethyluronium hexafluorophosphate), or HATU (0-(7-azabenzotriazol-1-yl)-N,N,N′,N′-tetramethyluronium hexafluorophosphate), an activator of the type of 1-hydroxybenzotriazole (HOBt) and N-hydroxysuccinimide to give the NHS ester of the label.

The fluorescent labels may include, without limitation, ethidium bromide, SYBR Green, fluorescein isothiocyanate (FITC), rhodamine tetramethyl isotiol (TRIT), 5-carboxyfluorescein, 6-carboxyfluorescein, fluorescein, HEX (6-carboxy-2′,4,4′,5′,7,7′-hexachlorofluorescein), Oregon Green 488, Oregon Green 500, Oregon Green 514, Joe (6-carboxy-4′,5′-dichloro-2′,7′-dimethoxyfluorescein), 5-carboxy-2′,4′,5′,7′-tetrachlorofluorescein, 5-carboxyrhodamine, rhodamine, tetramethylrhodamine (Tamra), Rox (carboxy-X-rhodamine), R6G (rhodamine 6G), phthalocyanines, azometazinas, cyanines (Cy2, Cy3 and Cy5), Texas Red, Princeston Red, BODIPY FL-Br2, BODIPY 530/550, BODIPY TMR, BODIPY 558/568, BODIPY 564/570, BODIPY 576/589, BODIPY 581/591, BODIPY TR, BODIPY 630/650, BODIPY 650/665, DABCYL, eosin, erythrosin, ethidium bromide, green fluorescent protein (GFP) and its analogues, inorganic-based fluorescent semiconductor nanocrystals (Quantum Dot), fluorescent labels based onlanthanide such as Eu3+ and Sm3+ and the like, rhodamine, phosphorus-lanthanides or FITC.

The enzymatic labels may include, without limitation, horseradish peroxidase, β-galactosidase, luciferase or alkaline phosphatase.

The preferred labeling include, but are not limited to, fluorescein, a phosphatase such as alkaline phosphatase, biotin, avidin, a peroxidase such as horseradish peroxidase and compounds related to biotin or compounds related to avidin (for example, streptavidin or ImmunoPure® NeutrAvidin available from Pierce, Rockford, Ill.).

In another particular embodiment, the antibody of the invention is labelled by conjugation to a first member of a binding pair. In a preferred embodiment, this modification is covalent biotinylation. The term “biotinylation”, as used herein, refers to the covalent attachment of biotin to a molecule (typically a protein). Biotinylation is performed using biotin reagents capable of conjugating to the side chain of the proteins, wherein said conjugation occurs primarily on the primary amino groups and thiol groups contained in the side chains of proteins. Suitable reagents for biotinylation of amino groups include molecules containing biotin and a group capable of reacting with amino groups such as succinimide esters, pentafluorophenyl ester or alkyl halides, wherein the biotin moiety and the reactive group separated by a spacer of any length.

In another particular embodiment, the antibody of the invention is labelled with metal ions such as gold (Au), including colloidal gold nanoparticles can be attached directly to the antibody via electrostatic interactions. In another particular embodiment, the colloidal gold nanoparticles are pre-coupled to biotin and can be covalently attached to the antibody.

Polynucleotides, Vectors and Host Cells

Polynucleotide sequences encoding monoclonal antibodies or fragments thereof, having high affinity and specificity for glycosylated ApoJ, as well as vectors and host cells carrying these polynucleotide sequences, are provided according to another aspect of the present invention

Therefore, in a second aspect the invention relates to polynucleotide encoding an antibody according to the first aspect of the invention.

The present invention provides nucleic acid molecules, specifically polynucleotides which, in some embodiments, encode one or more antibodies of the invention. The term “nucleic acid,” in its broadest sense, includes any compound and/or substance that comprise a polymer of nucleotides. These polymers are often referred to as polynucleotides. The term “polynucleotide” as referred to herein means a polymeric form of nucleotides of at least 10 bases in length. In certain embodiments, the bases may be ribonucleotides or deoxyribonucleotides or a modified form of either type of nucleotide. The term includes single and double stranded forms of DNA.

Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2 ‘-amino functionalization, and 2’-amino-a-LNA having a 2′-amino functionalization), ethylene nucleic acids (ENA), cyclohexenyl nucleic acids (CeNA) or hybrids or combinations thereof.

In one embodiment, linear polynucleotides encoding one or more antibody constructs of the present invention which are made using only in vitro transcription (IVT) enzymatic synthesis methods are referred to as “IVT polynucleotides.” Methods of making IVT polynucleotides are known in the art and are described in co-pending International Publication No. WO2013151666 filed Mar. 9, 2013 (Attorney Docket Number M300), the contents of which are incorporated herein by reference in their entirety.

Any of the polynucleotides described above may further include additional nucleic acids, encoding, e.g. a signal peptide to direct secretion of the encoded polypeptide, antibody constant regions as described herein, or other heterologous polypeptides as described herein. Also, as described in more detail elsewhere herein, the present invention includes compositions comprising one or more of the polynucleotides described above.

In one embodiment, the invention includes compositions comprising a first polynucleotide and second polynucleotide wherein said first polynucleotide encodes a VH domain as described herein and wherein said second polynucleotide encodes a VL domain as described herein.

The present invention also includes fragments of the polynucleotides of the invention. Additionally, polynucleotides that encode fusion polypolypeptides, Fab fragments, and other derivatives, as described herein, are also contemplated by the invention.

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., Bio Techniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding a glycosylated ApoJ antibody, or antigen-binding fragment, variant, or derivative thereof of the invention, may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other anti-glycosylated ApoJ antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the anti-glycosylated ApoJ antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al. (1990) Molecular Cloning, A Laboratory Manual (2nd ed.; Cold Spring Harbor Laboratory, Cold Spring Harbor, N.Y.) and Ausubel et al., eds. (1998) Current Protocols in Molecular Biology (John Wiley & Sons, NY), which are both incorporated by reference herein in their entireties), to generate antibodies having a different amino acid sequence, for example to create amino acid substitutions, deletions, and/or insertions.

A polynucleotide encoding an anti-glycosylated ApoJ antibody, or antigen-binding fragment, variant, or derivative thereof, can be composed of any polyribonucleotide or polydeoxyribonucleotide, which may be unmodified RNA or DNA or modified RNA or DNA. For example, a polynucleotide encoding anti-glycosylated ApoJ antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an anti-glycosylated ApoJ antibody, or antigen-binding fragment, variant, or derivative thereof can be composed of triple-stranded regions comprising RNA or DNA or both RNA and DNA.

A polynucleotide encoding an anti-glycosylated ApoJ antibody, or antigen-binding fragment, variant, or derivative thereof, may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

In one embodiment, the polynucleotides have a modular design to encode at least one of the antibodies, fragments or variants thereof described herein. As a non-limiting example, the polynucleotide construct may encode any of the following designs: (1) the heavy chain of an antibody, (2) the light chain of an antibody, (3) the heavy and light chain of the antibody, (4) the heavy chain and light chain separated by a linker, (5) the VHI, CHI, CH2, CH3 domains, a linker and the light chain and (6) the VHI, CHI, CH2, CH3 domains, VL region, and the light chain. Any of these designs may also comprise optional linkers between any domain and/or region.

In a particular embodiment the polynucleotide of the invention encodes a Fab, a F(ab)₂, a single domain antibody, a single chain variable fragment (scFv), or a nanobody.

In a preferred embodiment, the polynucleotide of the invention is selected from the group consisting of:
(i) a polynucleotide encoding an antibody according to the invention wherein the antibody is a single domain antibody, a single chain variable fragment (scFv), or a nanobody,
(ii) a polynucleotide encoding a polypeptide containing a heavy chain variable region according to Table 1,
(iii) a polynucleotide encoding a polypeptide containing light chain variable region according to Table 1 and,
(iv) a polycistronic polynucleotide encoding a polypeptide containing a light chain variable region according to Table 1 and a heavy chain variable region according to Table 1.

In a preferred embodiment, the polynucleotide according to the invention comprises the sequences as defined in SEQ ID NO; 143, 144, 145, 146, 147, 148, 149, 150 or 151.

In a related aspect the invention relates to an expression vector comprising the polynucleotide encoding the antibody of the invention

“Vector” includes shuttle and expression vectors and includes, e.g., a plasmid, cosmid, or phagemid. Typically, a plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in prokaryotic, e.g., bacterial, or eukaryotic cells. Suitable vectors are disclosed below.

For recombinant production of the antibody, the nucleic acid molecule encoding it is isolated and inserted into a replicable vector for further cloning (amplification of the DNA) or inserted into a vector in operable linkage with a promoter for expression. DNA encoding the antibody is readily isolated and sequenced using conventional procedures (e.g., by using oligonucleotide probes that are capable of binding specifically to nucleic acid molecules encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

The anti-glycosylated ApoJ antibody of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native anti-glycosylated ApoJ antibody signal sequence, the signal sequence is substituted by a prokaryotic signal sequence selected, for example, from the group of the alkaline phosphatase, penicillinase, 1pp, or heat-stable enterotoxin II leaders. For yeast secretion the native signal sequence may be substituted by, e.g., the yeast invertase leader, oc factor leader (including Saccharomyces and Kluyveromyces cc-factor leaders), or acid phosphatase leader, the C albicans glucoamylase leader, or the signal described in WO 90/13646. In mammalian cell expression, mammalian signal sequences as well as viral secretory leaders, for example, the herpes simplex gD signal, are available. The DNA for such precursor region is ligated in reading frame to DNA encoding the anti-glycosylated ApoJ antibody.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 p plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the anti-glycosylated ApoJ antibody nucleic acid, such as DHFR, thymidine kinase, metallothionein-I and -11, preferably primate metallothionein genes, adenosine deaminase, ornithine decarboxylase, etc. For example, cells transformed with the DHFR selection gene are first identified by culturing all of the transformants in a culture medium that contains methotrexate (Mtx) a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells particularly wild-type hosts that contain endogenous DHFR, transformed or co-transformed with DNA sequences encoding anti-glycosylated ApoJ antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4 Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 pm circular plasmid pKDI can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Expression and cloning vectors usually contain a promoter that is recognized by the host organism and is operably linked to the anti-glycosylated ApoJ antibody nucleic acid. Promoters suitable for use with prokaryotic hosts include the phoA promoter, P-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the anti-glycosylated ApoJ antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors. Examples of suitable promoter sequences for use with yeast hosts include the promoters for 3-phosphoglycerate kinase or other glycolytic enzymes, such as enolase, glyceraldehyde phosphate dehydrogenase, hexokinase, pyruvate decarboxylase, phosphofructokinase, glucose phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde phosphate dehydrogenase, and enzymes responsible for maltose and galactose utilization. Suitable vectors and promoters for use in yeast expression are further described in EP 73,657. Yeast enhancers also are advantageously used with yeast promoters.

Anti-glycosylated ApoJ antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian, Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human P-interferon cDNA in mouse cells under the control of a thymidine kinase promoter from herpes simplex virus. Alternatively, the Rous Sarcoma Virus long terminal repeat can be used as the promoter.

Transcription of a DNA encoding the anti-glycosylated ApoJ antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the anti-anti-glycosylated ApoJ antibody-encoding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′ untranslated regions of eukaryotic or viral DNAs or cDNAs. These regions contain nucleotide segments transcribed as polyadenylated fragments in the untranslated portion of the mRNA encoding anti-glycosylated ApoJ antibody. One useful transcription termination component is the bovine growth hormone polyadenylation region. See WO 94/11026 and the expression vector disclosed therein.

In another related aspect, the invention relates to a host cell comprising the polynucleotide or the vector of the invention.

The term “host cell”, as used herein, refers to a cell into which a nucleic acid of the invention, such as a polynucleotide or a vector according to the invention, has been introduced and is capable of expressing the micropeptides of the invention. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The term includes any cultivatable cell that can be modified by the introduction of heterologous DNA. Preferably, a host cell is one in which the polynucleotide of the invention can be stably expressed, post-translationally modified, localized to the appropriate subcellular compartment, and made to engage the appropriate transcription machinery. The choice of an appropriate host cell will also be influenced by the choice of detection signal.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W31 10 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half-life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for anti-glycosylated ApoJ antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-glycosilated ApoJ antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be utilized as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (tissue culture) has become a routine procedure. Examples of useful mammalian host cell lines are monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, 1413 8065); mouse mammary tumor (MMT 060562, ATCC CCLS 1); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-glycosylated ApoJ antibody production and cultured in conventional nutrient media modified as appropriate for inducing promoters, selecting transformants, or amplifying the genes encoding the desired sequences.

The host cells used to produce the anti-glycosylated ApoJ antibody of this invention may be cultured in a variety of media. Commercially available media such as Ham's FIO (Sigma), Minimal Essential Medium (MEM)(Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Compositions

In a third aspect the invention relates to a composition comprising at least two antibodies as defined in the first aspect of the invention or any of the above mentioned specific realizations or embodiments.

The term “composition”, as used herein, relates to a material composition that comprises any of the above-mentioned antibodies in any proportion and quantity, as well as any product resulting, directly or indirectly, from the combination of the different antibodies in any quantity thereof.

In a preferred embodiment the w/w ratio of the antibodies forming part of the composition of the invention is typically within the range from approximately 0.01:1 to 100:1. Suitable ratios include without limitation for example, 0.05:1, 0.1:1, 0.5:1, 1:1, 1:2, 1:3, 1:4, 1:5, 1:6, 1:7, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 100:1, 95:1, 90:1, 85:1, 80:1, 75:1, 70:1, 65:1, 60:1, 55:1, 50, 45:1, 40:1, 35:1, 30:1, 25:1, 20:1, 15:1, 10:1, 9:1, 8:1, 7:1, 6:1, 5:1, 4:1, 3:1, 2:1, 1:0.5, 1:0.1 and 1:0.05.

In a still preferred embodiment the w/w ratio of the antibodies forming part of the composition of the invention is 1:1.

Those skilled in the art will observe that the composition may be formulated as a single formulation or may be presented as separate formulations of each of the antibodies, which may be combined for joint use as a combined preparation. The composition may be a kit-of-parts wherein each of the components is individually formulated and packaged.

In a particular embodiment the composition of the invention is characterized in that one of the antibodies is the Ag2G-17 antibody.

In another embodiment, the composition of the invention comprises:

(i) the Ag2G-17 and the Ag6G-1 antibodies,

(ii) the Ag2G-17 and the Ag6G-11 antibodies,

(iii) the Ag2G-17 and the Ag7G-19 antibodies,

(iv) the Ag2G-17 and the Ag1G-11 antibodies,

(v) the Ag2G-17 and the Ag7G-17 antibodies,

(vi) the Ag2G-17 and the Ag4G-6 antibodies,

(vii) the Ag2G-17 and the Ag3G-4 antibodies or

(viii) the Ag2G-17 and the Ag5G-17 antibodies.

Method for Detection of Glycosylated Apo J

In a fourth aspect the invention relates to a method (hereinafter first method of the invention), for the determination of glycosylated Apo J in a sample comprising the steps of:
(i) Contacting the sample with an antibody of the invention or with a composition of the invention under conditions adequate for the formation of a complex between the antibody and the glycosylated Apo J present in the sample,
(ii) Determining the amount of complex formed in step (i).

In general, the immunobinding methods include obtaining a sample suspected of containing glycosylated ApoJ protein and contacting the sample with a composition capable of selectively binding or detecting the glycosylated ApoJ protein, under conditions effective to allow the formation of immunocomplexes.

The sample may be any sample that is suspected of containing the glycosylated ApoJ protein, such as, for example, a tissue section or specimen, a homogenized tissue extract, a cell, an organelle, separated and/or purified forms of any of the above antigen-containing compositions, or any biological fluid, including blood, serum and plasma. Preferably, the sample suspected of containing the glycosylated ApoJ protein is blood, serum or plasma.

Contacting the chosen biological sample with the antibody of the invention under effective conditions and for a period of time sufficient to allow the formation of immune complexes is generally a matter of simply adding the antibody composition to the sample and incubating the mixture for a period of time long enough for the antibodies to form immune complexes.

“Under conditions adequate for the formation of a complex” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” or “adequate” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The determination of the amount of complex formed may be done in a number of ways. In a preferred embodiment, the antibody is labelled, and binding determined directly. For example, this may be done by attaching the glycosylated ApoJ protein to a solid support, adding the labelled antibody (for example a fluorescent label), washing off excess reagent, and determining whether the label is present on the solid support. Various blocking and washing steps may be utilized as is known in the art.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241. Of course, one may find additional advantages through the use of a secondary binding ligand such as a second antibody and/or a biotin/avidin ligand binding arrangement, as is known in the art.

In a preferred embodiment, the determination of the complex in step (ii) of the first method of the invention is carried out using an anti-ApoJ antibody.

In a still preferred embodiment, the antibody used in step (i) or the antibodies within the composition used in step (i) of the first method of the invention are immobilized.

As the person skilled in the art will understand that there is a wide range of conventional assays that can be used in the present invention which use an antibody of the invention that is not labelled (primary antibody) and an antibody of the invention that is labelled (secondary antibody); these techniques include Western blot or immunoblot, ELISA (Enzyme-Linked Immunosorbent Assay), RIA (Radioimmunoassay), competitive EIA (Competitive Enzyme Immunoassay), DAS-ELISA (Double Antibody Sandwich-ELISA), immunocytochemical and immunohistochemical techniques, flow cytometry or multiplex detection techniques based on using protein microspheres, biochips or microarrays which include the antibody of the invention. Other ways of detecting and quantifying glycosylated ApoJ using the antibody of the invention include affinity chromatography techniques, ligand binding assays or lectin binding assays.

It will also be understood that antibodies that are not labelled need to be detected with an additional reagent, for example, a secondary antibody that is labelled, which will be labelled. This is particularly useful in order to increase the sensibility of the detection method, since it allows the signal to be amplified.

In addition, the detection of the antibody can also be carried out by detecting changes in the physical properties in the sample that occur as a result of the binding of the antibody to its cognate antigen. These assays include determining a transmission-related parameter in a sample, which are known in the art. The term “transmission-related parameter”, as used herein, relates to a parameter indicating or correlating with the ratio of transmitted light versus incident light of a sample or to a parameter derived therefrom.

In an embodiment, a transmission-related parameter is determined by turbidimetry or by nephelometry.

Turbidimetry, as used herein, refers to the measurement of light-scattering species in solution by means of a decrease in intensity of the incident beam after it has passed through solution. For turbidimetric assays, the change in the amount of light absorbed (inverse of amount transmitted) can be related to the amount of agglutination which occurs. Hence, the amount of analyte (the species causing agglutination) in the sample can be easily determined.

Nephelometry, as used herein, refers to a technique for measuring the light-scattering species in solution by means of the light intensity at an angle away from the incident light passing through the sample. Nephelometric assays present an indirect method of measurement of the amount of analyte in a sample by measuring the amount of light scattered or reflected at a given angle (typically 90) from the origin. In the presence of the protein antigen, the antibody reacts with the antigen, and a precipitation reaction begins. The measurement is taken early in this precipitation reaction time sequence. A quantitative value is obtained by comparison with a standard curve, which has been established previously. In order to increase the sensitivity of the detection, the antibody can be adsorbed or covalently attached to polymeric microspheres. In this way, a greater signal is produced with less reagent.

The detection method based on turbidimetry or nephelometry according to the present disclosure works with all known agglutination tests with and without microparticles enhancement. Typically used within the present disclosure is a “microparticle-enhanced light scattering agglutination tests” which is also called “particle-enhanced turbidimetric immunoassays” (PETIA). Agglutination-based immunoassays are routinely used in clinical diagnostics for the quantitation of serum proteins, therapeutic drugs and drugs of abuse on clinical chemistry analyzers, because they have the benefits of being quasi-homogeneous assays which do not require any separation or wash step. To enhance the optical detection between the antigen to be detected and the specific antibody in the reaction mixture, the antibody may be linked to suitable particles. Thereby, the antigen reacts and agglutinates with the particles which are coated with antibody. With increasing amount of antibody, the agglutination and the size of the complexes are increasing, leading further to a change of light scattering.

In another embodiment, the binding of the antibody to its cognate antigen can be detected by Surface plasmon resonance (SPR).

As used herein, SPR refers to a phenomenon that the intensity of a reflected light decreases sharply at a particular angle of incidence (i.e., an angle of resonance) when a laser beam is irradiated to a metal thin film. SPR is a measurement method based on the phenomenon described above and is capable of assaying a substance adsorbed on the surface of the metal thin film, which is a sensor, with high sensitivity. According to the present invention, for example, the target substance in the sample can then be detected by immobilizing one or more antibodies according to the present invention on the surface of the metal thin film beforehand, allowing the sample to pass through the surface of the metal thin film, and detecting the difference of the amount of the substance adsorbed on the surface of the metal thin film resulting from the binding of the antibody and the target antigen, between before and after the sample passes therethrough.

Diagnostic Methods for Ischemia Tissue Damage

In a fifth aspect, the invention relates to a method for the diagnosis of ischemia or ischemic tissue damage in a subject comprising determining in a sample of said subject the levels of glycosylated Apo J using an antibody as defined in the first aspect of the invention, a composition according to the third aspect of the invention or using the method as defined in the fourth aspect of the invention, wherein decreased levels of glycosylated Apo J with respect to a reference value are indicative that the patient suffers ischemia or ischemic tissue damage.

In the context of the present invention, the term “diagnosis” relates to the ability to discriminate between samples from patients with myocardial ischemia or ischemic tissue damage associated thereto and samples from individuals who have not suffered this injury and/or damage, when applied a method as disclosed herein. This detection as it is understood by one skilled in the art is not intended to be 100% correct for all the samples. However, it requires that a statistically significant number of samples analyzed are classified correctly. The amount that is statistically significant can be set by an expert in the field by using different statistical tools, for example, but not limited by the determination of confidence intervals, p value determination, Student's t test and discriminating function Fisher. Preferably, the confidence intervals are at least 90%, at least 95%, at least 97%, at least 98% or less than 99%. Preferably, the p value is less than 0.05, 0.01, 0.005 or 0.0001. Preferably, the present invention can correctly detect ischemia or ischemic damage in at least 60%, at least 70%, by at least 80%, or at least 90% of the subjects of a particular group or population tested.

The term “ischemia” is used herein interchangeably with “ischemic event” and refers to any situation resulting from a decrease or interruption of blood flow to an organ or tissue. Ischemia may be transient or permanent.

The expressions “ischemic tissue damage”, “ischemic tissue injury,” “tissue damage due to ischemia,” “tissue damage associated with ischemia,” “tissue damage as a result of ischemia,” “tissue damaged caused by ischemia,” and “ischemic-damaged tissue” refers to morphological, physiological, and/or molecular damage to an organ or tissue or cell as a result of a period of ischemia.

In one embodiment, the damage caused by ischemia is a damage of the cardiac tissue. In a still more preferred embodiment, the damage to the cardiac tissue is caused by myocardial ischemia.

The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

In yet another embodiment, the myocardial ischemia is acute myocardial ischemia or microvascular angina.

The term “microvascular angina”, as used herein, refers to a condition resulting from inadequate blood flow through the small cardiac blood vessels.

In one embodiment, the damage caused by ischemia is a damage of the cerebral tissue. In another embodiment, the damage to the cerebral tissue is caused by ischemic stroke. The term “ischemic stroke” refers to a sudden loss of brain function caused by a blockage or a blood vessel to the brain (resulting in the lack of oxygen to the brain), characterized by loss of muscular control, diminution or loss of sensation or consciousness, dizziness, slurred speech, or other symptoms that vary with the extent and the severity of the damage to the brain, also called cerebral accident, or cerebrovascular accident. The term “cerebral ischemia” (or “stroke”) also refers to a deficiency in blood supply to the brain, often resulting in a lack of oxygen to the brain.

In a further embodiment, the patient is suspected to have suffered an ischemic event.

The term “subject”, or “individual” or “animal” or “patient” includes any subject, particularly a mammalian subject, for whom therapy is desired. Mammalian subjects include humans, domestic animals, farm animals, and zoo or pet animals such as dogs, cats, guinea pigs, rabbits, rats, mice, horses, cattle, cows, and so on. In a preferred embodiment of the invention, the subject is a mammal. In a more preferred embodiment of the invention, the subject is a human.

The term “sample” or “biological sample”, as used herein, refers to biological material isolated from a subject. The biological sample contains any biological material suitable for detecting levels of glycosylated forms of a given protein, e.g. Apo J. The sample can be isolated from any suitable tissue or biological fluid such as, for example blood, saliva, plasma, serum, urine, cerebrospinal liquid (CSF) or feces. In a particular embodiment of the invention, the sample is a tissue sample or a biofluid. In a more particular embodiment of the invention, the biofluid is selected from the group consisting of blood, serum or plasma.

Preferably, the sample which is used for the determination of the levels of the different glycosylated forms of Apo J is the same type of sample used for determining the reference value in case that the determination is done in relative terms. By way of an example, if the determination of glycosylated Apo J is carried out in a plasma sample, then a plasma sample will also be used to determine the reference value. If the sample is a biofluid, then the reference sample will also be determined in the same type of biofluid, e.g. blood, serum, plasma, cerebrospinal fluid.

The term “decreased levels” or “low levels”, in relation to the levels of glycosylated Apo J relates to any level of expression of glycosylated Apo J detected using the antibodies according to the invention in a sample lower than the reference value. Thus, glycosylated Apo J expression levels are considered to be decreased or to be lower than its reference value when it is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more lower than its reference value.

The diagnostic method of the invention comprises comparing the levels obtained in the subject under study with a reference value, whereby decreased levels of glycosylated Apo J with respect to a reference value are indicative that the patient suffers ischemia or ischemic tissue damage.

The term “reference value”, as used herein, relates to a predetermined criteria used as a reference for evaluating the values or data obtained from the samples collected from a subject. The reference value or reference level can be an absolute value; a relative value; a value that has an upper or a lower limit; a range of values; an average value; a median value; a mean value; or a value as compared to a particular control or baseline value. A reference value can be based on an individual sample value, such as for example, a value obtained from a sample from the subject being tested, but at an earlier point in time. The reference value can be based on a large number of samples, such as from population of subjects of the chronological age matched group, or based on a pool of samples including or excluding the sample to be tested. In one embodiment, the reference value corresponds to the levels of glycosylated Apo J residues determined in a healthy subject, whereby a healthy subject is understood as a subject that shows no ischemic tissue damage at the moment the levels of glycosylated Apo J are determined and that, preferably, shows no history of ischemic damage.

In another embodiment, the reference value corresponds to an average or mean level of the corresponding biomarker determined from a pool of samples obtained from a group of patients who are well documented from the clinical point of view, and who present no disease, particularly who are not suffering from ischemic tissue damage, particularly not suffering from ischemic myocardial damage or ischemic cerebral damage. In said samples, the expression levels can be determined, for example by means of the determination of the average expression level in a reference population.

In the determination of the reference value, it is necessary to take into consideration some characteristics of the type of sample, such as age, gender, the physical state or other characteristics of the patient. For example, the reference sample can be obtained from identical amounts of a group of at least 2, at least 10, at least 100 to more than 1000 individuals, such that the population is statistically significant.

In a preferred embodiment, the diagnostic method according to the invention is carried out using the Ag2G-17 antibody, the Ag3G-4 antibody, the Ag4G-6 antibody, the Ag5G-17 antibody, the Ag6G-1 antibody, the Ag6G-11 antibody, the Ag7G-17 antibody, the Ag7G-19 antibody.

In another embodiment, the diagnostic method of the invention is carried out by using a composition comprising several antibodies according to the invention so that the value used for comparison is the aggregated value of the binding to each of the antibodies used. In preferred embodiments, the detection of glycosylated ApoJ is carried out using a composition selected from the group of:
(i) a composition comprising the Ag2G-17 and the Ag6G-1 antibodies,
(ii) a composition comprising the Ag2G-17 and the Ag6G-11 antibodies,
(iii) a composition comprising the Ag2G-17 and the Ag7G-19 antibodies,
(iv) a composition comprising the Ag2G-17 and the Ag1G-11 antibodies,
(v) a composition comprising the Ag2G-17 and the Ag7G-17 antibodies,
(vi) a composition comprising the Ag2G-17 and the Ag4G-6 antibodies,
(vii) a composition comprising the Ag2G-17 and the Ag3G-4 antibodies or
(viii) a composition comprising the Ag2G-17 and the Ag5G-17 antibodies.

It will be understood that the reference value used for the diagnosis of patients according to the diagnostic method of the invention is a value obtained from the same type of sample and the same antibody as those which are being considered in the diagnosis. Accordingly, if the diagnostic method is carried out by determining the levels of glycosylated Apo J using the Ag2G-17 antibody, then the reference value used in the diagnosis is also the expression level of glycosylated Apo J detected in using the same antibody. Similarly, if the diagnostic method is carried out by determining the levels of glycosylated Apo J using a composition of several antibodies according to the invention, then the reference value used in the diagnosis is also the expression level of glycosylated Apo J detected in using the same composition, as the case may be, obtained from a healthy subject or from a pool of samples as defined above.

In another embodiment, if the biomarker is determined in order to diagnose myocardial tissue damage, the reference value will be the levels of the same biomarker from a healthy subject who does not show myocardial tissue damage and who preferably has no record of suffering myocardial tissue damage. If the reference value is the average level of the same biomarker obtained from a pool of samples from subjects, then the subjects from which the pool of samples is prepared are subjects who do not show myocardial tissue damage and who preferably have no record of suffering myocardial tissue damage.

In another embodiment, if the biomarker is determined in order to diagnose cerebral tissue damage, the reference value will be the levels of the same biomarker from a healthy subject who does not show cerebral tissue damage and who preferably has no record of suffering cerebral tissue damage. If the reference value is the average level of the same biomarker obtained from a pool of samples from subjects, then the subjects from which the pool of samples are obtained are subjects who do not show cerebral tissue damage and who preferably have no record of suffering cerebral tissue damage.

The reference value used in the diagnostic method of the invention can be optimized in order to obtain a desired specificity and sensitivity.

In a preferred embodiment, the determination of the levels of glycosylated Apo J is carried out before a detectable increase in necrosis marker can be detected in the sample. In a preferred embodiment, the necrosis marker is either T-troponin or CK. In yet another embodiment, the determination of the levels of glycosylated Apo J is carried out in a sample obtained within 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 30 hours, 40 hours, 50 hours or more of the onset of the symptoms of ischemia damage. In a preferred embodiment, in the case of myocardial ischemic damage, symptoms are usually chest pain, shortness of breath, diaphoresis, weakness, light-headedness, nausea, vomiting, and palpitations. In one embodiment, the patient is a pre-AMI patient.

In another embodiment, the determination of the levels of the glycosylated ApoJ according to the diagnostic method of the invention is carried out in a sample from the patient which has been obtained before the patient has been administered with any medicament aimed at reducing ischemia or reducing the ischemic tissue damage. In one embodiment, in the case of myocardial tissue damage, the determination of the levels of the glycosylated forms of Apo J is carried out in a sample from the patient which has been obtained before the patient has been treated with statins, anti-platelets and/or anti-coagulants.

In a still preferred embodiment, the determination is carried out within the first 6 hours after the onset of the suspected ischemic event, before the raising of the levels of at least one necrotic marker and/or before the patient has received any treatment for the suspected ischemic event.

Method for the Prognosis of Patients Having Suffered Ischemic Damage

In a sixth aspect the invention relates to a method for predicting the progression of ischemia in a patient having suffered an ischemic event or for determining the prognosis of a patient having suffered an ischemic event, comprising determining in a sample of said patient the levels of glycosylated Apo J using an antibody as defined in the first aspect of the invention, a composition according to the third aspect of the invention or using the method as defined in the fourth aspect of the invention, wherein decreased levels of glycosylated Apo J with respect to a reference value are indicative that the ischemia is progressing or of a poor prognosis of the patient.

In a preferred embodiment, the prognostic method according to the invention is carried out using the Ag1G-11 antibody, the Ag2G-17 antibody, the Ag3G-4 antibody, the Ag4G-6 antibody, the Ag5G-17 antibody, the Ag6G-1 antibody, the Ag6G-11 antibody, the Ag7G-17 antibody, the Ag7G-19 antibody.

In another embodiment, the prognostic method of the invention is carried out by using a composition comprising several antibodies according to the invention so that the value used for comparison is the aggregated value of the binding to each of the antibodies used. In preferred embodiments, the detection of glycosylated ApoJ is carried out using a composition selected from the group of:
(i) a composition comprising the Ag2G-17 and the Ag6G-1 antibodies,
(ii) a composition comprising the Ag2G-17 and the Ag6G-11 antibodies,
(iii) a composition comprising the Ag2G-17 and the Ag7G-19 antibodies,
(iv) a composition comprising the Ag2G-17 and the Ag1G-11 antibodies,
(v) a composition comprising the Ag2G-17 and the Ag7G-17 antibodies,
(vi) a composition comprising the Ag2G-17 and the Ag4G-6 antibodies,
(vii) a composition comprising the Ag2G-17 and the Ag3G-4 antibodies or
(viii) a composition comprising the Ag2G-17 and the Ag5G-17 antibodies.

In the context of the present invention, the term “predicting the progression” relates to the ability to predict the course of the disease after suffering ischemia or ischemic tissue damage associated thereto when applied a method as disclosed herein. This detection, as understood by one skilled in the art, is not intended to be 100% correct for all the samples. However, it requires that a statistically significant number of analyzed samples have to be classified correctly. The amount that is statistically significant can be set by an expert in the field by using different statistical tools, for example, but not limited by the determination of confidence intervals, p value determination, Student's t test and discriminating function Fisher. Preferably, the confidence intervals are at least 90%, at least 95%, at least 97%, at least 98% or less than 99%. Preferably, the p value is less than 0.05, 0.01, 0.005 or 0.0001. Preferably, the present invention can correctly detect ischemia or ischemic damage in at least 60%, at least 70%, by at least 80%, or at least 90% of the subjects of a particular group or population tested.

In the context of the present invention, the term “determining the prognosis” is used interchangeably with “prognosis” relates to the ability to predict the outcome of patients after suffering myocardial or cerebral ischemia or ischemic tissue damage associated thereto when applied a method as disclosed herein. This detection as understood by one skilled in the art is not intended to be 100% correct for all the samples. However, it requires that a statistically significant number of analyzed samples are classified correctly. The amount that is statistically significant can be set by an expert in the field by using different statistical tools, for example, but not limited by the determination of confidence intervals, p value determination, Student's t test and discriminating function Fisher. Preferably, the confidence intervals are at least 90%, at least 95%, at least 97%, at least 98% or less than 99%. Preferably, the p value is less than 0.05, 0.01, 0.005 or 0.0001. Preferably, the present invention can correctly detect ischemia or ischemic damage in at least 60%, at least 70%, by at least 80%, or at least 90% of the subjects of a particular group or population tested.

In a preferred embodiment, the ischemic event is a myocardial ischemic event. In a still preferred embodiment, the myocardial ischemic event is a ST-elevation myocardial infarction.

In one embodiment, the prognosis of the patient is determined as the risk of 6 months recurrence. In the case of determining the risk of 6 months recurrence, it will be understood that recurrence refers to a second ischemic event occurring within the first 6 months after the first ischemic event. In one embodiment, the second ischemic event is of the same type as the first ischemic event, i.e. the first ischemic event is a myocardial ischemia and prognosis is determined as the risk that the patient suffers a second myocardial ischemic event. In another embodiment, the second ischemic event is of a different type as the first ischemic event, i.e. if the first ischemic event is a myocardial ischemia, then prognosis is determined as the risk that the patient suffers a cerebral ischemic event or vice versa, if the first ischemic event is a cerebral ischemic event, then prognosis is determined as the risk that the patient suffers a myocardial ischemic event.

In a still preferred embodiment, the prognosis of the patient is determined as the risk of 6 months recurrence, the risk of in hospital mortality or the risk of 6-months mortality.

In another embodiment, the prognosis of the patient is determined as the risk of in-hospital mortality.

In a preferred embodiment, the prognosis of the patient is determined as the risk of 6-months mortality.

The term “reference value”, when referred to the prognostic method of the invention, relates to a predetermined criteria used as a reference for evaluating the values or data obtained from the samples collected from a subject. The reference value or reference level can be an absolute value; a relative value; a value that has an upper or a lower limit; a range of values; an average value; a median value; a mean value; or a value as compared to a particular control or baseline value. A reference value can be based on an individual sample value, such as for example, a value obtained from a sample from the subject being tested, but at an earlier point in time. The reference value can be based on a large number of samples, such as from population of subjects of the chronological age matched group, or based on a pool of samples including or excluding the sample to be tested. In one embodiment, the reference value corresponds to the levels of glycosylated Apo J determined in a subject who has suffered an ischemic event and in which the ischemia has not progressed or who has had a good progression. In the case of progression determined as the risk of 6 months recurrence, the reference value can be taken as the glycosylated Apo J levels in a sample from a patient taken at the moment of the ischemic event but wherein the patient has not suffered any further ischemic event at least 6 months, 7 months, 8 months, 9 months, 10 month, 11 months, 12 months, 18 months, 24 months, 36 months, 48 months or more after the first ischemic event. In another embodiment, when the progression is determined as the risk of in hospital mortality, the reference value can be taken as the levels of glycosylated Apo J in a patient at the moment of the ischemic event but wherein the patient has been released from the hospital. In the case of progression determined as the risk of 6 months mortality, the reference value can be taken as the glycosylated Apo J levels in a sample from a patient taken at the moment of the ischemic event but wherein the patient is still alive at least 6 months, 7 months, 8 months, 9 months, 10 month, 11 months, 12 months, 18 months, 24 months, 36 months, 48 months or more after the ischemic event.

In another embodiment, the reference value corresponds to an average or mean level of the corresponding biomarker determined from a pool of samples obtained from a group of patients who are well documented from the clinical point of view, and who, after having suffered an ischemic event, have shown a good prognosis as defined in the previous paragraph. In said samples, the expression levels can be determined, for example by means of the determination of the average expression level in a reference population. In the determination of the reference value, it is necessary to take into consideration some characteristics of the type of sample, such as age, gender, the physical state and other characteristics of the patient. For example, the reference sample can be obtained from identical amounts of a group of at least 2, at least 10, at least 100 to more than 1000 individuals, such that the population is statistically significant.

It will be understood that the reference value used for the prognosis of patients according to the prognostic method of the invention is a value obtained from the same type of sample and using the same antibodies or compositions of antibodies as those used in the sample from the patient being analysed. Accordingly, if the prognostic method is carried out by determining the levels of glycosylated Apo J using the Ag2G-17 antibody, then the reference value used in the prognosis is also the expression level of glycosylated Apo J detected in using the same antibody. Similarly, if the prognosis method is carried out by determining the levels of glycosylated Apo J using a composition of several antibodies according to the invention, then the reference value used in the prognosis is also the expression level of glycosylated Apo J detected in using the same composition, as the case may be, obtained from a healthy subject or from a pool of samples as defined above.

In another embodiment, if the biomarker is determined in order to determine the prognosis of a patient having suffered a myocardial tissue damage, the reference value will be the levels of the same biomarker from a subject who, after having suffered a myocardial ischemic event, has shown a good prognosis according to any of the criteria defined above (lack of recurrence of ischemic event after 6 months, no in-hospital death or mortality after 6 months). If the reference value is the average level of the same biomarker obtained from a pool of samples from subjects, then the subjects from which the pool of samples is prepared are subjects who, after having suffered a myocardial ischemic event, have shown a good prognosis according to any of the criteria defined above (lack of recurrence of ischemic event after 6 months, no in-hospital death or mortality after 6 months)

In another embodiment, if the biomarker is determined in order to determine the prognosis of cerebral tissue damage, the reference value will be the levels of the same biomarker from a subject who, after having suffered a cerebral ischemic event, has shown a good prognosis according to any of the criteria defined above (lack of recurrence of ischemic event after 6 months, no in-hospital death or mortality after 6 months). If the reference value is the average level of the same biomarker obtained from a pool of samples from subjects, then the subjects from which the pool of samples is prepared are subjects who, after having suffered a cerebral ischemic event, have shown a good prognosis according to any of the criteria defined above (lack of recurrence of ischemic event after 6 months, no in-hospital death or mortality after 6 months).

The reference value used in the prognostic method of the invention can be optimized in order to obtain a desired specificity and sensitivity.

Risk Stratification Method of the Invention

The authors of the present invention have also shown that the levels of glycosylated Apo J determined using the antibodies and compositions according to the invention is also a useful biomarker for determining the risk that a patient who suffers stable coronary artery disease (CAD) suffers a recurrent ischemic event. This method allows the stratification of patients according to the risk that they suffer ischemic events and thus, is useful for assigning specific preventive therapies to the patients depending on the risk.

In a seventh aspect the invention relates to a method for determining the risk that a patient suffering from stable coronary disease suffers a recurrent ischemic event comprising determining in a sample of said patient the levels of glycosylated Apo J using an antibody as defined in the first aspect of the invention, a composition according to the third aspect of the invention or using the method as defined in the fourth aspect of the invention, wherein decreased levels of glycosylated Apo J with respect to a reference value are indicative that the patient shows an increased risk of suffering a recurrent ischemic event.

In a preferred embodiment, the risk stratification method according to the invention is carried out using the Ag1G-11 antibody, the Ag2G-17 antibody, the Ag3G-4 antibody, the Ag4g-6 antibody, the Ag5G-17 antibody, the Ag6G-1 antibody, the Ag6G-11 antibody, the Ag7G-17 antibody, the Ag7g-19 antibody.

In another embodiment, the risk stratification method of the invention is carried out by using a composition comprising several antibodies according to the invention so that the value used for comparison is the aggregated value of the binding to each of the antibodies used. In preferred embodiments, the detection of glycosylated ApoJ is carried out using a composition selected from the group of:
(i) a composition comprising the Ag2G-17 and the Ag6G-1 antibodies,
(ii) a composition comprising the Ag2G-17 and the Ag6G-11 antibodies,
(iii) a composition comprising the Ag2G-17 and the Ag7G-19 antibodies,
(iv) a composition comprising the Ag2G-17 and the Ag1G-11 antibodies,
(v) a composition comprising the Ag2G-17 and the Ag7G-17 antibodies,
(vi) a composition comprising the Ag2G-17 and the Ag4G-6 antibodies,
(vii) a composition comprising the Ag2G-17 and the Ag3G-4 antibodies or
(viii) a composition comprising the Ag2G-17 and the Ag5G-17 antibodies.

In the context of the present invention, the term “determining the risk” or “risk stratification” relates to the ability to determine the risk or probability of: a) suffering additional clinical complications of patients after suffering myocardial or cerebral ischemia or ischemic tissue damage associated thereto, and/or b) benefiting from a specific treatment for myocardial or cerebral ischemia or ischemic tissue damage associated thereto when applied a method as disclosed herein. This detection as it is understood by one skilled in the art is not intended to be 100% correct for all the samples. However, it requires that a statistically significant number of samples analyzed are classified correctly. The amount that is statistically significant can be set by an expert in the field by using different statistical tools, for example, but not limited by the determination of confidence intervals, p value determination, Student's t test and discriminating function Fisher. Preferably, the confidence intervals are at least 90%, at least 95%, at least 97%, at least 98% or less than 99%. Preferably, the p value is less than 0.1, 0.05, 0.01, 0.005 or 0.0001. Preferably, the present invention can correctly detect ischemia or ischemic damage in at least 60%, at least 70%, by at least 80%, or at least 90% of the subjects of a particular group or population tested.

The term “stable coronary disease” and “stable coronary heart disease” have the same meaning and are used interchangeable. Both terms include the medical condition stable coronary artery disease (SCAD). “Stable” in the context of the terms “stable cardiovascular disease”, “stable coronary disease” or “stable coronary heart disease” is defined as any conditions of diagnosed cardiovascular disease in the absence of acute cardiovascular events. Hence, e.g. stable coronary disease defines the different evolutionary phases of coronary disease, excluding the situations in, which coronary artery thrombosis dominates clinical presentation (acute coronary syndrome). Patients suffering from SCAD are defined by one or more of the following conditions: stable angina pectoris with positive ECG stress test or positive myocardial scintigraphy or stenosis of >50 percent of coronary artery, history of acute coronary syndrome, history of coronary revascularization, under treatment by anti-platelets, anti-coagulants and/or statins at a stable dose for at least 3 months.

In a preferred embodiment, patients suffering from stable coronary disease had suffered an acute coronary syndrome prior to the stable coronary disease. In preferred embodiments, the patient suffering from stable coronary disease had suffered an acute coronary syndrome at least 1 month, 2 months, 3 months, 4 months, 5 months, 6 months, 7 months, 8 months, 9 months, 10 months, 11 months, 12 months, 18 months, 24 months, 36 months, 48 months, 60 months or more prior to the stable coronary disease.

The term “reference value”, when referred to the risk stratification method of the invention, relates to a predetermined criteria used as a reference for evaluating the values or data obtained from the samples collected from a subject. The reference value or reference level can be an absolute value; a relative value; a value that has an upper or a lower limit; a range of values; an average value; a median value; a mean value; or a value as compared to a particular control or baseline value. A reference value can be based on an individual sample value, such as for example, a value obtained from a sample from the subject being tested, but at an earlier point in time. The reference value can be based on a large number of samples, such as from population of subjects of the chronological age matched group, or based on a pool of samples including or excluding the sample to be tested. In one embodiment, the reference value corresponds to the levels of glycosylated Apo J determined in a subject who suffers stable coronary disease but who has not suffered any recurrent ischemic event. In this case, the suitable patients from which the reference value can be determined are patients who have suffered stable coronary disease and who have not suffered ischemic recurrent events for at least 6 months, 7 months, 8 months, 9 months, 10 month, 11 months, 12 months, 18 months, 24 months, 36 months, 48 months or more after the onset of the stable coronary disease.

In another embodiment, the reference value corresponds to an average or mean level of the corresponding biomarker determined from a pool of samples obtained from a group of patients who are well documented from the clinical point of view, and who suffer stable coronary disease but who have not suffered a recurrent ischemic event for at least 6 months, 7 months, 8 months, 9 months, 10 month, 11 months, 12 months, 18 months, 24 months, 36 months, 48 months or more after the onset of the stable coronary disease. In said samples, the expression levels can be determined, for example by means of the determination of the average expression level in a reference population. In the determination of the reference value, it is necessary to take into consideration some characteristics of the type of sample, such as age, gender, the physical state and the like of the patient. For example, the reference sample can be obtained from identical amounts of a group of at least 2, at least 10, at least 100 to more than 1000 individuals, such that the population is statistically significant.

It will be understood that the reference value used for the risk stratification of according to method of the invention is a value obtained from the same type of sample and using the same antibodies or compositions of antibodies as those used in the sample from the patient being analysed. Accordingly, if the risk stratification method is carried out by determining the levels of glycosylated Apo J using the Ag2G-17 antibody, then the reference value used in the risk stratification is also the expression level of glycosylated Apo J detected in using the same antibody. Similarly, if the risk stratification method is carried out by determining the levels of glycosylated Apo J using a composition of several antibodies according to the invention, then the reference value used in the risk stratification is also the expression level of glycosylated Apo J detected in using the same composition, as the case may be, obtained from a healthy subject or from a pool of samples as defined above.

In a preferred embodiment, the patient suffering from stable coronary disease had suffered acute coronary syndrome prior to the stable coronary disease.

In a still preferred embodiment, the recurrent ischemic event is an acute coronary syndrome, a stroke or a transient ischemic event.

In a seventh aspect the invention relates to the use of an antibody according to the first aspect of the invention or of a composition according to the third aspect of the invention for the diagnosis of ischemia or ischemic tissue damage in a patient, for determining the progression of ischemia in a patient having suffered an ischemic event, for the prognosis of a patient having suffered an ischemic event or for determining the risk that a patient suffering from stable coronary disease suffers a recurrent ischemic event.

In an eight aspect the invention relates to the use of an antibody according to the first aspect of the invention or of a composition according to the third aspect of the invention for the diagnosis of ischemia or ischemic tissue damage in a patient, for determining the progression of ischemia in a patient having suffered an ischemic event, for the prognosis of a patient having suffered an ischemic event or for determining the risk that a patient suffering from stable coronary disease suffers a recurrent ischemic event.

The invention will be described by way of the following examples which are to be considered as merely illustrative and not limitative of the scope of the invention.

### EXAMPLES

Materials and Methods

Development of Specific Monoclonal Antibodies (MAbs) Against Apo J-GlcNAc

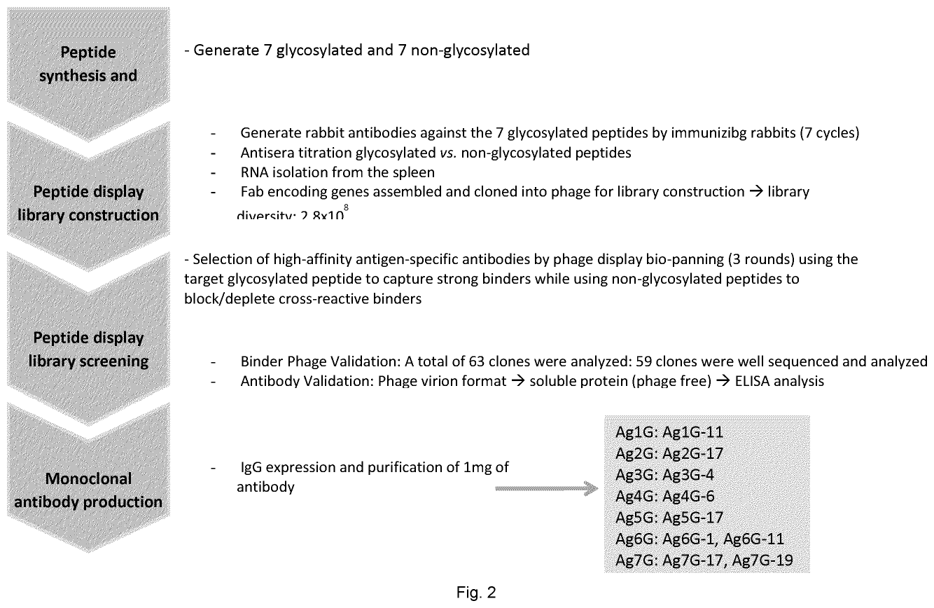

Specific monoclonal antibodies against 7 glycosylated peptides containing the 7 glycosylated sites in the Apo J sequence have been developed by phage display FIG. 2. Specifically, one antibody against each specific site and two additional clones for sites 6 and 7 have been developed.

Validation of MAbs for the Quantification of Different Apo J-GlcNAc Forms for Ischemia Detection

Patient Population

The validation study comprised a group of patients with a new onset ST-elevation myocardial infarction (STEMI) that were admitted at the emergency room within the first 6 hours after the onset of the pain and showed negative conventional troponin T (cTn-T) levels at admission (excluding subacute myocardial infarction) with a subsequent rise above the 99th percentile upper reference limit after the first blood sampling (ischemia pre-AMI).

A group of healthy donors without any previous manifestation of cardiovascular disease was used as control group. Demographic and clinical characteristics of the control group and ischemia pre-AMI patients are shown in Table 2.

TABLE 2
Patients included in the validation study. Values are
expressed as mean and SEM unless stated.

| | STEMI-patients | Controls | |
|---|---|---|---|
| | (N = 38) | (N = 144) | P-value |
| Age | 61 ± 2 | 63 ± 1 | 0.170 |
| Females/Males (N) | 10/28 | 73/71 | 0.007 |
| Risk factors (%) | | | |
| DL | 50 | 65 | 0.08 |
| DM | 24 | 10 | 0.03 |
| HTA | 50 | 42 | 0.357 |
| TB | 39 | 24 | 0.05 |
| Background medication (%) | | | |
| ASA | 11 | 8 | 0.529 |
| IECA/ARAII | 18 | 28 | 0.211 |
| Statins | 18 | 51 | 0.0004 |
| Beta-blockers | 8 | 7 | 0.840 |
| Ca-antagonists | 8 | 8 | 0.930 |
| KILLIP (%) | | | |
| I | 87 | — | — |
| II | 10 | — | — |
| III | 0 | — | — |
| IV | 3 | — | — |

The Ethics Committee of the Santa Creu i Sant Pau Hospital approved the project and the studies were conducted according to the principles of Helsinki's Declaration. All participants gave written informed consent to take part in the study.

Sample Collection and Preparation

Freshly drawn venous blood samples from patients and healthy individuals were collected to prepare serum that was aliquoted and stored at −80° C.

Quantification of Different Apo J-GlcNAc Forms with Specific MAbs

The levels of different Apo J-GlcNAc forms in serum samples of ischemia pre-AMI patients and healthy controls were measured with an immunoassay based on the different MAbs against the different Apo J-GlcNAc residues. This methodology is based on:
1) a first step in which Apo J-GlcNAc is immobilized by the specific binding of the specific glycosylation residue within the Apo J protein sequence to each specific MAb;
2) a second step, in which immobilized Apo J is detected with a specific commercially available biotinylated antibody against Apo J protein sequence (ab69644, Abcam); and
3) a final step in which the amount of the specific immobilized glycosylated Apo J form is further quantified by a reporter system consisting in a streptavidin-HRP conjugate (21130, Pierce) reacting with the biotinylated antibody.

Quantification of Total Apo J-GlcNAc Levels with Lectins

The levels of total Apo J-GlcNAc forms in serum samples of ischemia pre-AMI patients and healthy controls were measured with a lectin-based immunoassay. This methodology is based on:
1) a first step in which proteins are bound to immobilized D. stramonium lectin,
2) a second step, in which Apo J is detected with a monoclonal or polyclonal antibody against Apo J protein sequence, and
3) a final step in which the amount of the immobilized glycosylated Apo J form is further detected and quantified by a reporter system or molecule. This reporter system is based on a secondary antibody together with a reporter system such as biotin-streptavidin-HRP.

Statistical Analysis

Data are expressed as mean and standard error except when indicated. N indicates the number of subjects tested. Statistical analyses were performed with Stat View 5.0.1 software. Student's t-test was used for comparison between groups. Chi-square test (χ2) or Fisher's exact test, when any of the expected values was <5, was used for categorical variables. Receiver operating characteristic (ROC) curves (to assess the discriminating power of selected variables) were performed with IBM SPSS Statistics v19.0. A P value <0.05 was considered significant.

Results

Monoclonal Antibodies Development

FIG. 2 shows a schematic diagram of the methodological approach used to develop the specific monoclonal antibodies against the s even Apo J-GlcNAc glycosylation sites.

1. Immune Library Construction

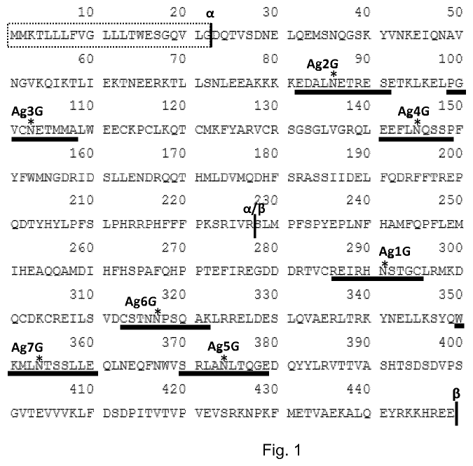

Five types of peptides for each target glycosylation site (FIG. 1) have been synthesised in different formats (naked, BSA-conjugated and biotin-conjugated glycosylated peptides, naked peptides and biotin-conjugated non-glycosylated peptides). Then each BSA-conjugated glycosylated peptide has been used to immunize separate rabbit 7 times. Afterwards, the bleeding was performed, and the antisera titration was conducted with the biotin-conjugated glycosylated peptides to monitor the immune response, using biotin-conjugated peptides as positive controls. The peptide sequences for titration are listed in Table 3.

TABLE 3
Peptides for titration

| Peptide Number | Peptide Sequence |
|---|---|
| Ag1G | Bio-REIRHN(GlcNAc)STGC |
| Ag1 | Bio-REIRHNSTGC |
| Ag2G | Bio-EDALN(GlcNAc)ETRES |
| Ag2 | Bio-EDALNETRES |
| Ag3G | Bio-PGVCN(GlcNAc)ETMMA |
| Ag3 | Bio-PGVCNETMMA |
| Ag4G | Bio-EEFLN(GlcNAc)QSSP |
| Ag4 | Bio-EEFLNQSSP |
| Ag5G | Bio-SRLAN(GlcNAc)LTQGE |
| Ag5 | Bio-SRLANLTQGE |
| Ag6G | Bio-CSTNN(GlcNAc)PSQAK |
| Ag6 | Bio-CSTNNPSQAK |
| Ag7G | Bio-WKMLN(GlcNAc)TSSLE |
| Ag7 | Bio-WKMLNTSSLE |

The titers of anti-serum against all seven biotin-conjugated glycosylated peptides were higher than that of biotin-conjugated peptides, meaning that they could be used for antibody phage display library construction.

Then, the immune library was constructed. RNA was isolated from the spleen. The Vκ, VH, Cκ and CH1 were amplified by PCR and the Fab encoding genes were assembled and cloned into pCDisplay-11 for library construction. The library diversity reached 2.8×10⁸ as summarized in Table 4. QC colony PCR was conducted to assay the inserting ratio of the end library.

TABLE 4
Summary of the end library

| Diversity | Positive insertion rate | Titer (CFU) |
|---|---|---|
| 2.8 × 10⁸ | 14/20 | 2.9 × 10¹³ |

QC colony PCR was conducted to assay the inserting ratio of the end library, the positive rate was 14/20. Then the DNA was sequenced.

2. Library Screening

After the rabbit/human chimeric Fab library has been constructed successfully, the screening phase was carried out. Two rounds of biopanning were completed. In order to eliminate the binders that binds to non-glycosylation sites and non-specific glycosylation sites, a mix of the seven biotin-conjugated non-glycosylated peptides (Ag1, Ag2, Ag3, Ag4, Ag5, Ag6, Ag7) and a mix of the biotin-conjugated glycosylation peptides (AgnG, n=1-7, except the target peptide) were performed against the phage library first. Then the positive target was screened to enrich.

In order to reduce background, the experimental conditions were optimized as follows. (1) Unblocked streptavidin coated wells and blocked streptavidin coated wells were performed against the phage library first before the (Ag1, Ag2, Ag3, Ag4, Ag5, Ag6, Ag7) mix and (AgnG, n=1-7, except the target peptide) mix. (2) The positive target was screened to enrich. (3) The blocking buffer was changed, and the blocking time was also extended. (4) The washing times and time are extended.

After three rounds of biopanning against the seven targets (Ag1G, Ag2G, Ag3G, Ag4G, Ag5G, Ag6G, Ag7G), polyclonal phage ELISA was carried out using the outputs of 1st, 2nd and 3rd round. Then, polyclonal phage ELISA was carried out again after scheme optimization. In order to further reduce the non-specific binders, the (Ag1, Ag2, Ag3, Ag4, Ag5, Ag6, Ag7) mix and (AgnG, n=1-7, except the target peptide) mix were performed against the outputs of 1st, 2nd and 3rd round before incubation.

3. Binder Validation

Twenty clones from 3rd-P of biopanning against seven targets were randomly selected. QC monoclonal phage ELISA was conducted using phages in culture medium with precipitation. The results showed that 17/59 unique clones were identified for six targets (Ag1G, Ag2G, Ag4G, Ag5G, Ag6G, Ag7G) in total. Among of them, 2 clones for Ag1G, 2 clones for Ag2G, 4 clones for Ag4G, 2 clones for Ag5G, 3 clones for Ag6G, and 4 clones for Ag7G. However, for the target of Ag3G, no intact antibody sequence was identified in the first round. Besides, the leftover 42/59 clones were the same sequence, which was an incomplete antibody sequence with a partial of the VH domain and was found from all the targets non-specifically.

In a second round, another 5 clones from Ag3G group were picked for sequencing. After the analysis of the sequences of those 5 clones, 4 of them clones were the same non-specific sequence (as happened with the other six targets). One positive clone was identified, and the antibody sequence was intact.

We then performed the soluble ELISA for the selected clones to the seven targets. One positive clone for each target (from Ag1G to Ag5G) and two positive clones for targets

Ag6G and Ag7G were identified in monoclonal phage ELISA. The expression vectors were constructed and soluble ELISAs using the cell lysates were carried out

4. IgGs Production and Validation

Nine clones (Ag1G-11, Ag2G-17, Ag3G-4, Ag4G-6, Ag5G-17, Ag6G-1, Ag6G-11, Ag7G-17, and Ag7G-19) in the form of IgG were produced and QC ELISA was conducted. Finally, all the 9 IgGs indicated consistent results with the former QC soluble ELISA. Moreover, the two IgGs against Ag6G showed more distinguish differences than other binders (to their corresponding target). Nomenclature of the monoclonal antibodies and corresponding binding glycosylation site are shown in Table 5.

TABLE 5
Specific ApoJ Glycosylation site detected by
the final produced clones by phage display

| Apo J sequence site | Apo J peptide No | MAb clones |
|---|---|---|
| 86 | Ag2G | Ag2G-17 |
| 103 | Ag3G | Ag3G-4 |
| 145 | Ag4G | Ag4G-6 |
| 291 | Ag1G | Ag1G-11 |
| 317 | Ag6G | Ag6G-1, Ag6G-11 |
| 354 | Ag7G | Ag7G-17, Ag7G-19 |
| 374 | Ag5G | Ag5G-17 |

Discriminating Ability for the Presence of Ischemia

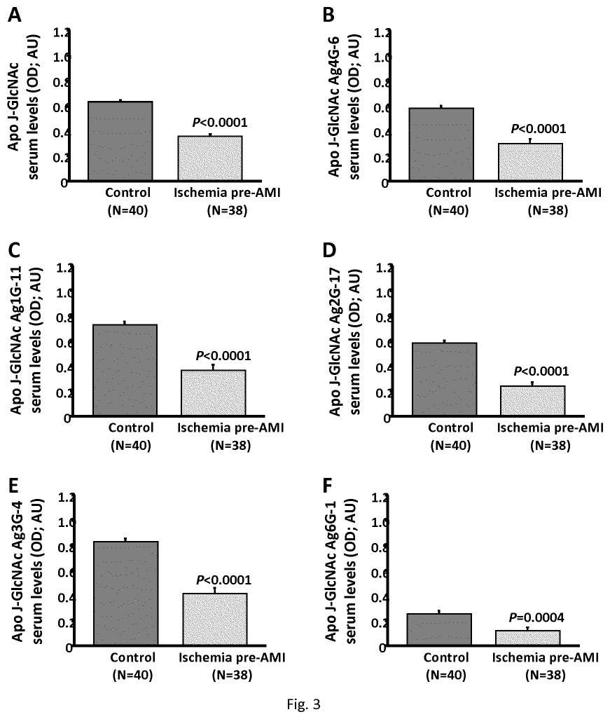

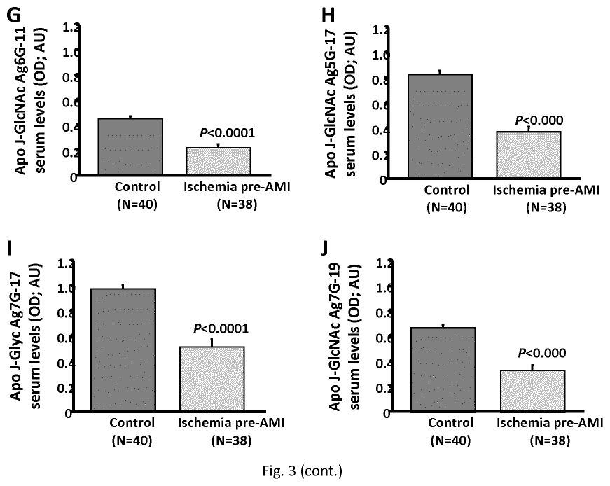

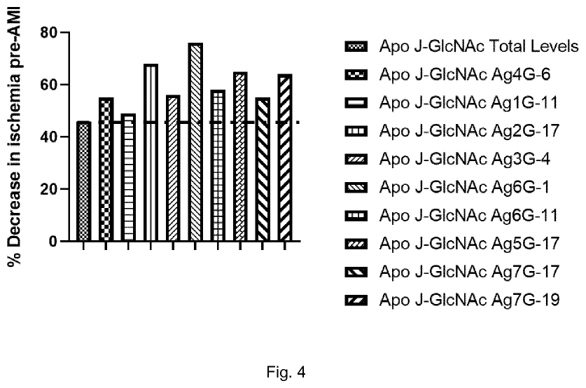

In order to test the discriminating ability of the detection of specific glycosylation residues of the Apo J sequence containing GlcNAc ELISA tests were run with each specific clone targeting the 7 different glycosylation sites on serum samples of ischemia pre-AMI patients (N=38) and healthy controls (N=40). Compared to the quantification of total Apo J-GlcNAc levels with the lectin-based immunoassay, the quantification of each Apo J-GlcNAc form with specific antibodies targeting each independent glycosylated residue showed a strongest decrease in ischemia pre-AMI patients compared to control subjects FIGS. 3 and 4 and Table 6.

TABLE 6
Retrospective analysis of samples.
Mean value of the optical density (OD) in arbitrary units
(AU) showing the intensity of Apo J-GlcNAc levels in serum samples of healthy controls and ischemia pre-
AMI patients measured: a) with the lectin-based immunoassay detecting total Apo J-GlcNAc levels (row in
grey) and b) with specific antibodies targeting each independent Apo J-GlcNAc glycosylated residue. The
detection with specific MAbs against each independent Apo J-GlcNAc glycosylated residue depicted a
strongest decrease in Apo J-GlcNAc levels in AMI patients in the early ischemic phase.

| | Control | Ischemia pre-AMI | % detection in | % Decrease in |
|---|---|---|---|---|
| | (N = 40; OD; AU) | (N = 38; OD; AU) | ischemia pre-AMI | ischemia pre-AMI |
| Apo J-GlcNAc total levels | 0.623 | 0.334 | 54 | 46 |
| Apo J-GlcNAc Ag4G-6 | 0.576 | 0.258 | 45 | 55 |
| Apo J-GlcNAc Ag1G-11 | 0.732 | 0.370 | 51 | 49 |
| Apo J-GlcNAc Ag2G-17 | 0.567 | 0.184 | 32 | 68 |
| Apo J-GlcNAc Ag3G-4 | 0.812 | 0.357 | 44 | 56 |
| Apo J-GlcNAc Ag6G-1 | 0.241 | 0.058 | 24 | 76 |
| Apo J-GlcNAc Ag6G-11 | 0.456 | 0.190 | 42 | 58 |
| Apo J-GlcNAc Ag5G-17 | 0.838 | 0.290 | 35 | 65 |
| Apo J-GlcNAc Ag7G-17 | 0.956 | 0.428 | 45 | 55 |
| Apo J-GlcNAc Ag7G-19 | 0.663 | 0.241 | 36 | 64 |

Specifically, the detection of Apo J-GlcNAc with MAbs against glycosylated residues 2 (clone Ag2G-17) and 6 (clone Ag6G-1) depicted the strongest decrease in Apo J-GlcNAc levels in AMI patients in the early ischemic phase. Moreover, Table 6 shows that all MAbs against Apo J with GlcNAc residues have a better discriminating ability for the detection of the decrease in Apo J-Glyc levels showing a higher % of decrease in Apo J-Glyc in ischemia pre-AMI patientes than lectins: 49-76 MAbs vs 46 lectins (FIG. 4).

C-statistics analysis revealed a high discriminating ability for the presence of an ischemic event of the detection of Apo J-GlcNAc levels in serum samples with specific antibodies targeting each independent Apo J-GlcNAc glycosylated residue. ROC analysis of individual MAbs depicted AUC (area under the curve) values between 0.751 and 0.918 with high percentages of sensitivity and specificity Table 7.

TABLE 7
C-statistics ROC analysis results individual MAbs.
C-statistics receiver operating curve (ROC) analysis and associated sensitivity and
specificity showing the discriminating ability for the presence of an ischemic event
of the detection of Apo J-GlcNAc levels in serum samples with specific antibodies
targeting each independent Apo J-GlcNAc glycosylated residue.

| Specific MAb against GlcNAc residue | AUC | 95% CI | P-value | Sensitivity | Specificity |
|---|---|---|---|---|---|
| Apo J-GlcNAc Ag4G-6 | 0.870 | 0.780-0.961 | <0.0001 | 93 | 76 |
| Apo J-GlcNAc Ag1G-11 | 0.886 | 0.802-0.969 | <0.0001 | 95 | 79 |
| Apo J-GlcNAc Ag2G-17 | 0.918 | 0.849-0.987 | <0.0001 | 98 | 82 |
| Apo J-GlcNAc Ag3G-4 | 0.885 | 0.800-0.969 | <0.0001 | 98 | 76 |
| Apo J-GlcNAc Ag6G-1 | 0.751 | 0.636-0.866 | <0.0001 | 75 | 79 |
| Apo J-GlcNAc Ag6G-11 | 0.886 | 0.803-0.969 | <0.0001 | 95 | 76 |
| Apo J-GlcNAc Ag5G-17 | 0.895 | 0.820-0.971 | <0.0001 | 95 | 76 |
| Apo J-GlcNAc Ag7G-17 | 0.849 | 0.752-0.946 | <0.0001 | 100 | 74 |
| Apo J-GlcNAc Ag7G-19 | 0.855 | 0.763-0.947 | <0.0001 | 90 | 76 |

AUC: area under the curve;
CI: confidence interval.

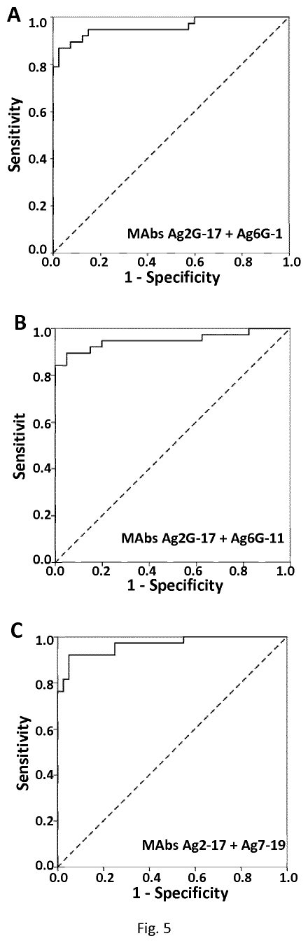

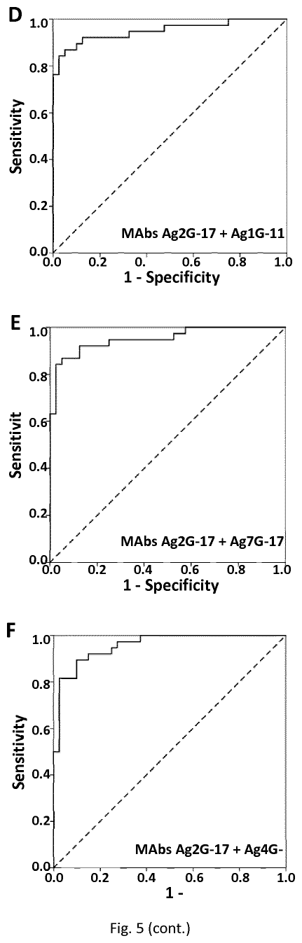

In order to test whether the combination of different glycosylated residues could increase the sensitivity and specificity of the Apo J-GlcNAc quantification method for the discrimination of the presence of ischemia ROC analysis of all the potential combinations of the individual MAbs measurements were carried out. FIG. 5 shows ROC curves of the 6 combinations that showed a high discriminating ability for the detection of the presence of an ischemic event. Importantly, the combination of the detection of different Apo J-GlcNAc forms with specific antibodies targeting independent glycosylated residues showed a higher discriminating ability for the detection of an ischemic event than the quantification of total Apo J-GlcNAc levels with the lectin-based immunoassay Table 8. Specifically, the combination of the detection of Apo J-GlcNAc serum levels with the clone Ag2G-17 in combination with the clones Ag6G-1, Ag6G-11, Ag7G-19 and Ag1G-11 depicted the maximum discriminating ability for the detection of ischemia (95% confidence interval reaching values of 1.000).

TABLE 8
C-statistics ROC analysis results MAbs combinations.
C-statistics receiver operating curve (ROC) analysis and associated sensitivity and
specificity showing the discriminating ability for the presence of an ischemic
event of the detection of Apo J-GlcNAc levels in serum samples.
Combinations of specific antibodies targeting independent Apo J-GlcNAc
glycosylated residues show a higher specificity for the detection of ischemia than
the quantification of total Apo J-GlcNAc levels with the lectin-based immunoassay.

| Mabs combinations | AUC | 95% CI | P-value | Sensitivity | Specificity |
|---|---|---|---|---|---|
| Apo J-GlcNAc total levels | 0.928 | 0.864-0.991 | <0.0001 | 97 | 79 |
| Apo J-GlcNAc Ag2G-17 + Ag6G-1 | 0.958 | 0.913-1.000 | <0.0001 | 90 | 92 |
| Apo J-GlcNAc Ag2G-17 + Ag6G-11 | 0.950 | 0.896-1.000 | <0.0001 | 90 | 95 |
| Apo J-GlcNAc Ag2-17 + Ag7-19 | 0.966 | 0.929-1.000 | <0.0001 | 92 | 95 |
| Apo J-GlcNAc Ag2G-17 + Ag1G-11 | 0.950 | 0.900-1.000 | <0.0001 | 92 | 87 |
| Apo J-GlcNAc Ag2G-17 + Ag7G-17 | 0.951 | 0.905-0.998 | <0.0001 | 92 | 87 |
| Apo J-GlcNAc Ag2G-17 + Ag4G-6 | 0.957 | 0.917-0.996 | <0.0001 | 92 | 87 |

AUC: area under the curve;
CI: confidence interval.

MAbs Targeting the 7 Different Glycosylation Sites in Apo J Show Improved Discriminating Ability of the Presence of Ischemia than Lectins Specifically Recognising the N-glycans Found in Apo J

In order to test the discriminating ability of the detection of specific glycosylation residues of the Apo J sequence containing GlcNAc ELISA tests were run with each specific clone targeting the 7 different glycosylation sites on serum samples of ischemia pre-AMI patients (N=38) and healthy controls (N=40). The results of the quantification of total Apo J-GlcNAc levels with the D. Stramonium lectin-based immunoassay are shown in Table 9.

TABLE 9
Specificity of the lectin-based immunoassay.
Specificity values obtained with the C-statistics receiver
operating curve (ROC) analysis showing the discriminating
ability for the presence of an ischemic event of the detection
of Apo J-GlcNAc levels in serum samples with the lectin-
based in two cohorts of patients: ischemia pre-AMI
patients and STEMI patients.

| Discovery Study | STEMI Lectin |
|---|---|
| Lectin Specificity | Specificity* |
| 71 | 53/72 |

*53% for Triticum Vulgaris Lectin; 72% for Datura Stramonium Lectin.

Compared to the quantification of total Apo J-GlcNAc levels with the lectin-based immunoassay (Table 9), the quantification of each Apo J-GlcNAc form with specific antibodies targeting each independent glycosylated residue showed a strongest decrease in ischemia pre-AMI patients compared to control subjects (see FIGS. 3 and 4 and Table 6). Specifically, the detection of Apo J-GlcNAc with MAbs against glycosylated residues 2 (clone Ag2G-17) and 6 (clone Ag6G-1) depicted the strongest decrease in Apo J-GlcNAc levels in AMI patients in the early ischemic phase.

C-statistics analysis revealed a high discriminating ability for the presence of an ischemic event of the detection of Apo J-GlcNAc levels in serum samples with specific antibodies targeting each independent Apo J-GlcNAc glycosylated residue. ROC analysis of individual MAbs depicted AUC (area under the curve) values between 0.751 and 0.918 with higher percentages of specificity than lectins (74-82% MAbs vs. 53-72 lectins) (compare specificity values in the MAbs detection assay in Table 7 with specificity values in the lectin assay in Table 9).

MAbs Bind Native Apo J from Serum but not Other Heavily N-GlcNAc Glycosylated Proteins

MAbs specificity was also tested against other heavily N-GlcNAc glycosylated proteins (such as Albumin and Transferrin). For that purpose, 2 pg of either native Apo J protein purified from human plasma and serum, Albumin and Transferrin were loaded into a nitrocellulose membrane with a narrow-mouth pipette tip. After drying, non-specific sites were blocked by soaking in 5% BSA in TBS-T (1 hour at room temperature). Membrane was incubated for 30 minutes at room temperature with either Ag2G-17 or Ag6G-11 clones (as they were the ones that showed the best combination of specificity-sensitivity). After 3 washes with TBS-T, membrane was incubated with the secondary antibody conjugated with HRP for 30 min at room temperature. Finally, 3 washes with TBS-T (1×15 minutes and 2×5 minutes) were performed before washing with TBS (5 minutes). Membranes were incubated with Supersignal and exposed in ChemiDoc.

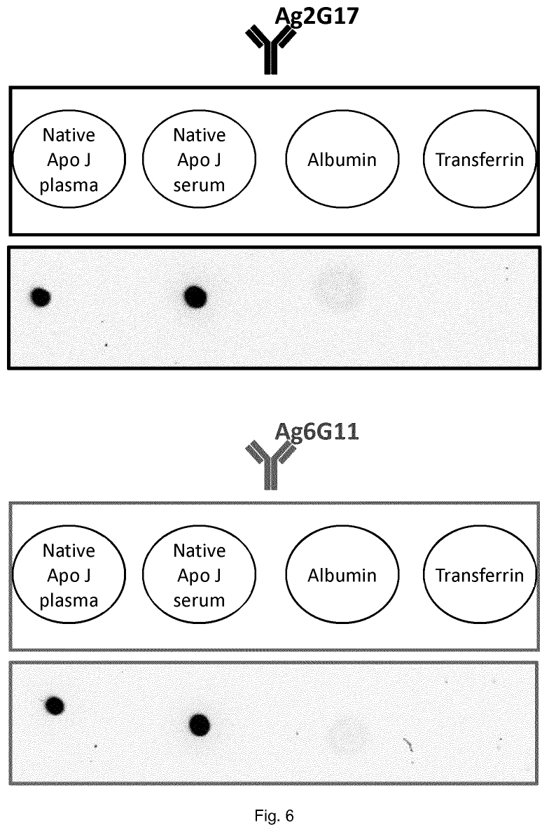

Monoclonal antibodies bind to native Apo J from plasma and serum but not to other heavily N-GlcNAc glycosylated proteins (such as Albumin and Transferrin), showing its specificity against glycosylated Apo J (FIG. 6). On the contrary, lectins, by definition, bind non-specifically to all the glycosylated proteins irrespectively of the aminoacidic sequence.

1. An antibody which specifically binds glycosylated ApoJ but which does not bind non-glycosylated ApoJ, wherein.
(i) the antibody specifically recognizes an epitope which comprises a N-glycosylation site within ApoJ and wherein said glycosylation site comprises a Asn residues selected from the group consisting of the Asn residues at positions 86, 103, 145, 291, 317, 354 or 374 with respect to the ApoJ precursor sequence as defined in the NCBI database entry with accession number NP_001822.3 or
(ii). the antibody specifically recognizes or has been generated using a peptide selected from the group consisting of SEQ ID NO: 118, 119, 120, 121, 122, 123 or 124, wherein the peptides are modified with N-acetylglucosamine residues at the Asn residues at position 5 in SEQ ID NO: 118, at position 5 in SEQ ID NO:119, position 5 a in SEQ ID NO:120, at position 6 in SEQ ID NO:121, at position 5 in SEQ ID NO:122, at position 5 in SEQ ID NO:123 or at position 5 in SEQ ID NO:124.

2. The antibody of claim 1 wherein the glycosylated Apo J is glycosylated Apo J containing N-acetylglucosamine (GlcNAc) residues or glycosylated Apo J containing N-acetylglucosamine (GlcNAc) and sialic acid residues.

3. The antibody of claim 1 comprising:
a) a light chain complementarity determining region 1 (VL-CDR1) comprising an amino acid sequence set forth in any one of SEQ ID NOs: 1, 6, 11, 16, 21, 26, 31, 36, 41 or a functionally equivalent variant thereof;
b) a light chain complementarity determining region 2 (VL-CDR2) comprising any of the amino acid sequences QAS, KAS, RAS, SAS, DAS, or a functionally equivalent variant thereof;
c) a light chain complementarity determining region 3 (VL-CDR3) comprising an amino acid sequence set forth in any one of SEQ ID NOs: 2, 7, 12, 17, 22, 27, 32, 37, 42 or a functionally equivalent variant thereof;
d) a heavy chain complementarity determining region 1 (VH-CDR1) comprising an amino acid sequence set forth in any one of SEQ ID NOs: 3, 8, 13, 18, 23, 28, 33, 38, 43 or a functionally equivalent variant thereof;
e) a heavy chain complementarity determining region 2 (VH-CDR2) comprising an amino acid sequence set forth in any one of SEQ ID NOs: 4, 9, 14, 19, 24, 29, 34, 39, 44 or a functionally equivalent variant thereof or
f) a heavy chain complementarity determining region 3 (VH-CDR3) comprising an amino acid sequence set forth in any one of SEQ ID NOs: 5, 10, 15, 20, 25, 30, 35, 40, 45 or a functionally equivalent variant thereof.

4. The antibody of claim 3 wherein:
(i) the VL-CDR1 comprises an amino acid sequence set forth in SEQ ID NO:6, the VL-CDR2 comprises the amino acid sequence KAS and the VL-CDR3 comprises an amino acid sequence set forth in SEQ ID NO: 7,
(ii) the VL-CDR1 comprises an amino acid sequence set forth in SEQ ID NO: 1, the VL-CDR2 comprises the amino acid sequence QAS and the VL-CDR3 comprises an amino acid sequence set forth in SEQ ID NO: 2,
(iii) the VL-CDR1 comprises an amino acid sequence set forth in SEQ ID NO: 11, the VL-CDR2 comprises the amino acid sequence RAS and the VL-CDR3 comprises an amino acid sequence set forth in SEQ ID NO: 12
(iv) the VL-CDR1 comprises an amino acid sequence set forth in SEQ ID NO: 16, the VL-CDR2 comprises the amino acid sequence QAS and the VL-CDR3 comprises an amino acid sequence set forth in SEQ ID NO: 17,
(v) the VL-CDR1 comprises an amino acid sequence set forth in SEQ ID NO: 21, the VL-CDR2 comprises the amino acid sequence SAS and the VL-CDR3 comprises an amino acid sequence set forth in SEQ ID NO: 22,
(vi) the VL-CDR1 comprises an amino acid sequence set forth in SEQ ID NO: 26, the VL-CDR2 comprises the amino acid sequence DAS and the VL-CDR3 comprises an amino acid sequence set forth in SEQ ID NO: 27,
(vii) the VL-CDR1 comprises an amino acid sequence set forth in SEQ ID NO: 31, the VL-CDR2 comprises the amino acid sequence SAS and the VL-CDR3 comprises an amino acid sequence set forth in SEQ ID NO: 32,
(viii) the VL-CDR1 comprises an amino acid sequence set forth in SEQ ID NO: 36, the VL-CDR2 comprises the amino acid sequence KAS and the VL-CDR3 comprises an amino acid sequence set forth in SEQ ID NO: 37
(ix) the VL-CDR1 comprises an amino acid sequence set forth in SEQ ID NO: 41, the VL-CDR2 comprises the amino acid sequence KAS and the VL-CDR3 comprises an amino acid sequence set forth in SEQ ID NO: 42,
(x) the VH-CDR1 comprises an amino acid sequence set forth in SEQ ID NO: 8, the VH-CDR2 comprises an amino acid sequence set forth in SEQ ID NO: 9 and the VH-CDR3 comprises an amino acid sequence set forth in SEQ ID NO: 10,
(xi) the VH-CDR1 comprises an amino acid sequence set forth in SEQ ID NO: 3, the VH-CDR2 comprises an amino acid sequence set forth in SEQ ID NO: 4 and the VH-CDR3 comprises an amino acid sequence set forth in SEQ ID NO: 5,
(xii) the VH-CDR1 comprises an amino acid sequence set forth in SEQ ID NO: 13, the VH-CDR2 comprises an amino acid sequence set forth in SEQ ID NO: 14 and the VH-CDR3 comprises an amino acid sequence set forth in SEQ ID NO: 15,
(xiii) the VH-CDR1 comprises an amino acid sequence set forth in SEQ ID NO: 18, the VH-CDR2 comprises an amino acid sequence set forth in SEQ ID NO: 19 and the VH-CDR3 comprises an amino acid sequence set forth in SEQ ID NO: 20,
(xiv) the VH-CDR1 comprises an amino acid sequence set forth in SEQ ID NO: 23, the VH-CDR2 comprises an amino acid sequence set forth in SEQ ID NO: 24 and the VH-CDR3 comprises an amino acid sequence set forth in SEQ ID NO: 25,
(xv) VH-CDR1 comprises an amino acid sequence set forth in SEQ ID NO: 28, the VH-CDR2 comprises an amino acid sequence set forth in SEQ ID NO: 29 and the VH-CDR3 comprises an amino acid sequence set forth in SEQ ID NO: 30,
(xvi) the VH-CDR1 comprises an amino acid sequence set forth in SEQ ID NO: 33, the VH-CDR2 comprises an amino acid sequence set forth in SEQ ID NO: 34 and the VH-CDR3 comprises an amino acid sequence set forth in SEQ ID NO: 35,
(xvii) the VH-CDR1 comprises an amino acid sequence set forth in SEQ ID NO: 38, the VH-CDR2 comprises an amino acid sequence set forth in SEQ ID NO: 39 and the VH-CDR3 comprises an amino acid sequence set forth in SEQ ID NO: 40 or
(xviii) the VH-CDR1 comprises an amino acid sequence set forth in SEQ ID NO: 43, the VH-CDR2 comprises an amino acid sequence set forth in SEQ ID NO: 44 and the VH-CDR3 comprises an amino acid sequence set forth in SEQ ID NO: 45.

5. The antibody of claim 4 wherein:
(i) the VL-CDR1 comprises an amino acid sequence set forth in SEQ ID NO: 6, the VL-CDR2 comprises the amino acid sequence KAS, the VL-CDR3 comprises an amino acid sequence set forth in SEQ ID NO: 7, the VH-CDR1 comprises an amino acid sequence set forth in SEQ ID NO: 8, the VH-CDR2 comprises an amino acid sequence set forth in SEQ ID NO: 9 and the VH-CDR3 comprises an amino acid sequence set forth in SEQ ID NO: 10,
(ii) the VL-CDR1 comprises an amino acid sequence set forth in SEQ ID NO: 1, the VL-CDR2 comprises the amino acid sequence QAS, the VL-CDR3 comprises an amino acid sequence set forth in SEQ ID NO: 2, the VH-CDR1 comprises an amino acid sequence set forth in SEQ ID NO: 3, the VH-CDR2 comprises an amino acid sequence set forth in SEQ ID NO: 4 and the VH-CDR3 comprises an amino acid sequence set forth in SEQ ID NO: 5,
(iii) the VL-CDR1 comprises an amino acid sequence set forth in SEQ ID NO: 1, the VL-CDR2 comprises the amino acid sequence RAS wherein the VL-CDR3 comprises an amino acid sequence set forth in SEQ ID NO: 12, the VH-CDR1 comprises an amino acid sequence set forth in SEQ ID NO: 13, the VH-CDR2 comprises an amino acid sequence set forth in SEQ ID NO: 14 and the VH-CDR3 comprises an amino acid sequence set forth in SEQ ID NO: 15,
(iv) the VL-CDR1 comprises an amino acid sequence set forth in SEQ ID NO: 16, the VL-CDR2 comprises the amino acid sequence QAS, the VL-CDR3 comprises an amino acid sequence set forth in SEQ ID NO: 17, the VH-CDR1 comprises an amino acid sequence set forth in SEQ ID NO: 18, the VH-CDR2 comprises an amino acid sequence set forth in SEQ ID NO: 19 and the VH-CDR3 comprises an amino acid sequence set forth in SEQ ID NO: 20,
(v) the VL-CDR1 comprises an amino acid sequence set forth in SEQ ID NO: 21, the VL-CDR2 comprises the amino acid sequence SAS, the VL-CDR3 comprises an amino acid sequence set forth in SEQ ID NO: 22, the VH-CDR1 comprises an amino acid sequence set forth in SEQ ID NO: 23, the VH-CDR2 comprises an amino acid sequence set forth in SEQ ID NO: 24 and the VH-CDR3 comprises an amino acid sequence set forth in SEQ ID NO: 25,
(vi) the VL-CDR1 comprises an amino acid sequence set forth in SEQ ID NO: 26, the VL-CDR2 comprises the amino acid sequence DAS, the VL-CDR3 comprises an amino acid sequence set forth in SEQ ID NO: 27, the VH-CDR1 comprises an amino acid sequence set forth in SEQ ID NO: 28, the VH-CDR2 comprises an amino acid sequence set forth in SEQ ID NO: 29 and the VH-CDR3 comprises an amino acid sequence set forth in SEQ ID NO: 30,
(vii) the VL-CDR1 comprises an amino acid sequence set forth in SEQ ID NO: 31, the VL-CDR2 comprises the amino acid sequence SAS, the VL-CDR3 comprises an amino acid sequence set forth in SEQ ID NO: 32, the VH-CDR1 comprises an amino acid sequence set forth in SEQ ID NO: 33, the VH-CDR2 comprises an amino acid sequence set forth in SEQ ID NO: 34 and the VH-CDR3 comprises an amino acid sequence set forth in SEQ ID NO: 35,
(viii) the VL-CDR1 comprises an amino acid sequence set forth in SEQ ID NO: 36, the VL-CDR2 comprises the amino acid sequence KAS, the VL-CDR3 comprises an amino acid sequence set forth in SEQ ID NO: 37, the VH-CDR1 comprises an amino acid sequence set forth in SEQ ID NO: 38, the VH-CDR2 comprises an amino acid sequence set forth in SEQ ID NO: 39 and the VH-CDR3 comprises an amino acid sequence set forth in SEQ ID NO: 40,
(ix) the VL-CDR1 comprises an amino acid sequence set forth in SEQ ID NO: 41, the VL-CDR2 comprises the amino acid sequence KAS, the VL-CDR3 comprises an amino acid sequence set forth in SEQ ID NO: 42, the VH-CDR1 comprises an amino acid sequence set forth in SEQ ID NO: 43, the VH-CDR2 comprises an amino acid sequence set forth in SEQ ID NO: 44 and the VH-CDR3 comprises an amino acid sequence set forth in SEQ ID NO: 45.

6. The antibody of claim 1 further comprising one or more of:
(i) a light chain framework 1 (VL-FR1) region amino acid sequence at least 90% identical to the amino acid sequence set forth in any one of SEQ ID NOs: 46, 54, 62, 70, 78, 86, 94, 102 or 110,
(ii) a light chain framework 2 (VL-FR2) region amino acid sequence at least 90% identical to the amino acid sequence set forth in any one of SEQ ID NOs: 47, 55, 63, 71, 79, 87, 95, 103 or 111,
(iii) a light chain framework 3 (VL-FR3) region amino acid sequence at least 90% identical to the amino acid sequence set forth in any one of SEQ ID NOs: 48, 56, 64, 72, 80, 88, 96, 104 or 112 and
(iv) a light chain framework 4 (VL-FR4) region amino acid sequence at least 90% identical to the amino acid sequence set forth in any one of SEQ ID NOs: 49, 57, 65, 73, 81, 89, 97, 105 or 113.

7. The antibody of claim 1 further comprising one or more of:
(i) a heavy chain framework 1 (VH-FR1) region amino acid sequence at least 90% identical to the amino acid sequence set forth in any one of SEQ ID NOs: 50, 58, 66, 74, 82, 90, 98, 106 or 114,
(ii) a heavy chain framework 2 (VH-FR2) region amino acid sequence at least 90% identical to the amino acid sequence set forth in any one of SEQ ID NOs: 51, 59, 67, 75, 83, 91, 99, 107 or 115,
(iii) a heavy chain framework 3 (VH-FR3) region amino acid sequence at least 90% identical to the amino acid sequence set forth in any one of SEQ ID NOs: 52, 60, 68, 76, 84, 92, 100, 108 or 116 and
(iv) a heavy chain framework 4 (VH-FR4) region amino acid sequence at least 90% identical to the amino acid sequence set forth in any one of SEQ ID NOs: 53, 61, 69, 77, 85, 93, 101, 109 or 117.

8. The antibody of claim 1 comprising:
i) a light chain domain defined by the amino acid sequence set forth in any one of SEQ ID NOs: 125, 126, 127, 128, 129, 130, 131, 132 or 133, and/or
ii) a heavy chain domain defined by the amino acid sequence set forth in any of SEQ ID Nos: 134, 135, 136, 137, 138, 139, 140, 141 or 142.

9. The antibody of claim 1 wherein the respective VL-FWR1, VL-CDR1, VL-FWR2, VL-CDR2, VL-FWR3, VL-CDR3, VL-FWR4, VH-FWR1, VH-CDR1, VH-FWR2, VH-CDR2, VH-FWR3, VH-CDR3 and VH-FWR4 regions of each antibody comprise the amino acid sequences set forth in Table 1.

10. The antibody of claim 1 which comprises at least one framework region derived from a human antibody framework region, which is humanized or which is super-humanized.

11. The antibody of claim 1 wherein the antibody is a Fab, a F(ab)₂, a single-domain antibody, a single chain variable fragment (scFv), or a nanobody.

12. The antibody according to claim 1 wherein the antibody is coupled to a detectable label.

13. (canceled)

14. A polynucleotide selected from the group consisting of:
(i) a polynucleotide encoding an antibody according to claim 1 wherein the antibody is a single domain antibody, a single chain variable fragment (scFv), or a nanobody,
(ii) a polynucleotide encoding a heavy chain variable region according to Table 1,
(iii) a polynucleotide encoding a light chain variable region according to Table 1 and,
(iv) a polycistronic polynucleotide encoding a light chain variable region according to Table 1 and a heavy chain variable region according to Table 1.

15. An expression vector comprising the polynucleotide according to claim 14.

16. A host cell comprising the polynucleotide according to any of claim 15.

17. A composition comprising at least two antibodies as defined in claim 1.

18. The composition according to claim 17 wherein one of the antibodies is the Ag2G-17 antibody.

19. The composition of claim 18 wherein the composition comprises:
(i) the Ag2G-17 and the Ag6G-1 antibodies,
(ii) the Ag2G-17 and the Ag6G-11 antibodies,
(iii) the Ag2G-17 and the Ag7G-19 antibodies,
(iv) the Ag2G-17 and the Ag1G-11 antibodies,
(v) the Ag2G-17 and the Ag7G-17 antibodies,
(vi) the Ag2G-17 and the Ag4G-6 antibodies,
(vii) the Ag2G-17 and the Ag3G-4 antibodies or
(viii) the Ag2G-17 and the Ag5G-17 antibodies.

20. A method for the determination of glycosylated Apo J in a sample comprising the steps of:
(i) Contacting the sample with an antibody according to claim 1 under conditions adequate for the formation of a complex between the antibody and the glycosylated Apo J present in the sample,
(ii) Determining the amount of complex formed in step (i).

21-22. (canceled)

23. A method selected from the group consisting of:
(a) a method for the diagnosis of ischemia or ischemic tissue damage in a subject comprising determining in a sample of said subject the levels of glycosylated Apo J using an antibody as defined in claim 1, wherein decreased levels of glycosylated Apo J with respect to a reference value are indicative that the patient suffers ischemia or ischemic tissue damage;
(b) a method for predicting the progression of ischemia in a patient having suffered an ischemic event or for determining the prognosis of a patient having suffered an ischemic event, comprising determining in a sample of said patient the levels of glycosylated Apo J using an antibody as defined in claim 1 wherein decreased levels of glycosylated Apo J with respect to a reference value are indicative that the ischemia is progressing or of a poor prognosis of the patient; and
(c) a method for determining the risk that a patient suffering from stable coronary disease suffers a recurrent ischemic event comprising determining in a sample of said patient the levels of glycosylated Apo J using an antibody as defined in claim 1 wherein decreased levels of glycosylated Apo J with respect to a reference value are indicative that the patient shows an increased risk of suffering a recurrent ischemic event.

24-35. (canceled)


SEQUENCE LISTING (167 sequences)

SEQ ID NO: 1 - protein, 8 aa, Artificial sequence
  CDR
QSVYNNNE

SEQ ID NO: 2 - protein, 12 aa, Artificial sequence
  CDR
QGIYLGSDWYDV

SEQ ID NO: 3 - protein, 8 aa, Artificial sequence
  CDR
GFSLSSYA

SEQ ID NO: 4 - protein, 7 aa, Artificial sequence
  CDR
IGVNGDT

SEQ ID NO: 5 - protein, 15 aa, Artificial sequence
  CDR
ARVRYPYYDTDAFDP

SEQ ID NO: 6 - protein, 6 aa, Artificial sequence
  CDR
QSIGNL

SEQ ID NO: 7 - protein, 12 aa, Artificial sequence
  CDR
QSYYAIASYGVA

SEQ ID NO: 8 - protein, 8 aa, Artificial sequence
  CDR
GFSLSRYP

SEQ ID NO: 9 - protein, 8 aa, Artificial sequence
  CDR
ISSGGWLT

SEQ ID NO: 10 - protein, 16 aa, Artificial sequence
  CDR
ARFGRYGNTDYYYFDL

SEQ ID NO: 11 - protein, 6 aa, Artificial sequence
  CDR
QSISTY

SEQ ID NO: 12 - protein, 15 aa, Artificial sequence
  CDR
QTAHDSSGRGTWGVI

SEQ ID NO: 13 - protein, 8 aa, Artificial sequence
  CDR
GFSLSSYG

SEQ ID NO: 14 - protein, 7 aa, Artificial sequence
  CDR
IDVSGSA

SEQ ID NO: 15 - protein, 12 aa, Artificial sequence
  CDR
ARGSPGYDANDL

SEQ ID NO: 16 - protein, 6 aa, Artificial sequence
  CDR
QSINGY

SEQ ID NO: 17 - protein, 10 aa, Artificial sequence
  CDR
QGDYYGWIRT

SEQ ID NO: 18 - protein, 8 aa, Artificial sequence
  CDR
GMDLSKYW

SEQ ID NO: 19 - protein, 7 aa, Artificial sequence
  CDR
IETGGSA

SEQ ID NO: 20 - protein, 6 aa, Artificial sequence
  CDR
GRWGDI

SEQ ID NO: 21 - protein, 8 aa, Artificial sequence
  CDR
ESVYVNNF

SEQ ID NO: 22 - protein, 12 aa, Artificial sequence
  CDR
AGYHEFNTDGNA

SEQ ID NO: 23 - protein, 8 aa, Artificial sequence
  CDR
GFSLSRHT

SEQ ID NO: 24 - protein, 7 aa, Artificial sequence
  CDR
ITYGGSA

SEQ ID NO: 25 - protein, 12 aa, Artificial sequence
  CDR
GRVGAYGAYYDL

SEQ ID NO: 26 - protein, 8 aa, Artificial sequence
  CDR
QSVRGNND

SEQ ID NO: 27 - protein, 12 aa, Artificial sequence
  CDR
QQGHRVEDVANA

SEQ ID NO: 28 - protein, 8 aa, Artificial sequence
  CDR
GFSLNSYY

SEQ ID NO: 29 - protein, 7 aa, Artificial sequence
  CDR
VSTDGSA

SEQ ID NO: 30 - protein, 15 aa, Artificial sequence
  CDR
ARDRGSDSYIDQLDL

SEQ ID NO: 31 - protein, 6 aa, Artificial sequence
  CDR
ESISSR

SEQ ID NO: 32 - protein, 9 aa, Artificial sequence
  CDR
LGTYSAIRT

SEQ ID NO: 33 - protein, 8 aa, Artificial sequence
  CDR
GFSLSTYN

SEQ ID NO: 34 - protein, 7 aa, Artificial sequence
  CDR
IYGSGSV

SEQ ID NO: 35 - protein, 11 aa, Artificial sequence
  CDR
ARMGSGWGFNI

SEQ ID NO: 36 - protein, 6 aa, Artificial sequence
  CDR
QSIDSY

SEQ ID NO: 37 - protein, 12 aa, Artificial sequence
  CDR
QCSHYGSNWLGP

SEQ ID NO: 38 - protein, 9 aa, Artificial sequence
  CDR
GFSLNTYYW

SEQ ID NO: 39 - protein, 7 aa, Artificial sequence
  CDR
INPDGTA

SEQ ID NO: 40 - protein, 12 aa, Artificial sequence
  CDR
ARLGSTGDNFNI

SEQ ID NO: 41 - protein, 8 aa, Artificial sequence
  CDR
QSVYSNSF

SEQ ID NO: 42 - protein, 10 aa, Artificial sequence
  CDR
QGGYVDWMRA

SEQ ID NO: 43 - protein, 8 aa, Artificial sequence
  CDR
GIDLSSNA

SEQ ID NO: 44 - protein, 7 aa, Artificial sequence
  CDR
INTNGKT

SEQ ID NO: 45 - protein, 15 aa, Artificial sequence
  CDR
ARGRWTGNTGWYIDL

SEQ ID NO: 46 - protein, 26 aa, Artificial sequence
  FWR
DPVLTQTPSPVSAAVGGTVTINCQAS

SEQ ID NO: 47 - protein, 17 aa, Artificial sequence
  FWR
LSWYQQKPGQPPKPLIY

SEQ ID NO: 48 - protein, 36 aa, Artificial sequence
  FWR
KLASGVPSRFKGSGSGTQFTLTISDLECDDAATYYC

SEQ ID NO: 49 - protein, 117 aa, Artificial sequence
  FWR
FGGGTEVVVKRTVAAPSVFIFPPSDEQLKSGTASVVCLLNNFYPREAKVQWKVDNALQSGNSQESVTEQDSKDSTYSLSSTLTLSRADYEKHKVYACEVTHQGLSSPVTKSFNRGEC

SEQ ID NO: 50 - protein, 24 aa, Artificial sequence
  FWR
QSLEESGGRLVTPGGSLTLTCTVS

SEQ ID NO: 51 - protein, 17 aa, Artificial sequence
  FWR
MSWVRQAPGKGLEWIGI

SEQ ID NO: 52 - protein, 36 aa, Artificial sequence
  FWR
YYASWAKGRFTISKTSTTVGLKITSPTTEDTATYFC

SEQ ID NO: 53 - protein, 126 aa, Artificial sequence
  FWR
WGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHH

SEQ ID NO: 54 - protein, 23 aa, Artificial sequence
  FWR
MTQTPASASEPVGGTVTIKCQAS

SEQ ID NO: 55 - protein, 17 aa, Artificial sequence
  FWR
LAWYQQKPGQPPKFLIY

SEQ ID NO: 56 - protein, 36 aa, Artificial sequence
  FWR
TLASGVSSQFKGSGSGTEFTLTISDLECADAATYYC

SEQ ID NO: 57 - protein, 117 aa, Artificial sequence
  FWR
FGAGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 25 aa, Artificial sequence
  FWR
QEQLKESGGRLVTPGGSLTLTCTVA

SEQ ID NO: 59 - protein, 16 aa, Artificial sequence
  FWR
MNVRQAPGKGLEWIGV

SEQ ID NO: 60 - protein, 36 aa, Artificial sequence
  FWR
FYANWAKGRFTISKTSTTVDLKITSPTTEDTTTYFC

SEQ ID NO: 61 - protein, 109 aa, Artificial sequence
  FWR
WGQGTLVTVSSASTKGPSVFPLAPSSKSTSVTVSWNSGALTSGVHTFPAVLQSSGLYSLSSVVTVPSSSLGTQTYICNVNHKPSNTKVDKKVEPKSCDKTSGSHHHHHH

SEQ ID NO: 62 - protein, 26 aa, Artificial sequence
  FWR
DVVMTQTPASVEAAVGGTVTIKCQAS

SEQ ID NO: 63 - protein, 17 aa, Artificial sequence
  FWR
LSWHQQKPGQPPKLLIY

SEQ ID NO: 64 - protein, 36 aa, Artificial sequence
  FWR
TLESGVPSRFKGSGSGTEFALTISDLECADAATYYC

SEQ ID NO: 65 - protein, 117 aa, Artificial sequence
  FWR
FGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 25 aa, Artificial sequence
  FWR
QEQLEESGGRLVTPGTPLTLTCAVS

SEQ ID NO: 67 - protein, 17 aa, Artificial sequence
  FWR
VSWVRQAPGKGLEYIGY

SEQ ID NO: 68 - protein, 36 aa, Artificial sequence
  FWR
YYASWARGRFTISRTSTTVDLKMTSLTTEDTATYFC

SEQ ID NO: 69 - protein, 131 aa, Artificial sequence
  FWR
WGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHHAAATE

SEQ ID NO: 70 - protein, 23 aa, Artificial sequence
  FWR
MTQTPASVEVAVGGTVTIKCQAS

SEQ ID NO: 71 - protein, 17 aa, Artificial sequence
  FWR
LAWYQQKPGQPPKLLIY

SEQ ID NO: 72 - protein, 36 aa, Artificial sequence
  FWR
TLASGVSSRFQGSGSGTEYTLTISGVQCDDAATYYC

SEQ ID NO: 73 - protein, 117 aa, Artificial sequence; identical to SEQ ID NO: 65
  FWR

SEQ ID NO: 74 - protein, 24 aa, Artificial sequence
  FWR
QQLEESGGRLVTPGTPLTLTCTAS

SEQ ID NO: 75 - protein, 17 aa, Artificial sequence
  FWR
MTWVRQAPGKGLEYIGI

SEQ ID NO: 76 - protein, 36 aa, Artificial sequence
  FWR
YYASWAKGRFTISRTSTTVDLKMISPTTEDTATYFC

SEQ ID NO: 77 - protein, 126 aa, Artificial sequence
  FWR
WGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHH

SEQ ID NO: 78 - protein, 26 aa, Artificial sequence
  FWR
DVVMTQTPSSKSVPVGDTVTINCQAS

SEQ ID NO: 79 - protein, 17 aa, Artificial sequence
  FWR
LSWYRQKPGQPPKRLIY

SEQ ID NO: 80 - protein, 36 aa, Artificial sequence
  FWR
TLASGVPSRFSGSGSGTQFTLTISDVVCDDAATYYC

SEQ ID NO: 81 - protein, 117 aa, Artificial sequence; identical to SEQ ID NO: 65
  FWR

SEQ ID NO: 82 - protein, 25 aa, Artificial sequence
  FWR
QEQLVESGGRLVTPGTPLILTCTAS

SEQ ID NO: 83 - protein, 17 aa, Artificial sequence
  FWR
MSWVRQAPGKGLEWIGY

SEQ ID NO: 84 - protein, 36 aa, Artificial sequence
  FWR
YSANWAKGRFTISRTSTTVDLKMNSLTTEDTATYFC

SEQ ID NO: 85 - protein, 130 aa, Artificial sequence
  FWR
WGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHHAPLR

SEQ ID NO: 86 - protein, 26 aa, Artificial sequence
  FWR
DPMLTQTPSSVSAAVGGTVTANCQSS

SEQ ID NO: 87 - protein, 17 aa, Artificial sequence
  FWR
LAWYQQKPGQPPKLLIY

SEQ ID NO: 88 - protein, 36 aa, Artificial sequence
  FWR
KLASGVPSRFKGSGSGTDFTLTISDLECADAATYYC

SEQ ID NO: 89 - protein, 117 aa, Artificial sequence; identical to SEQ ID NO: 65
  FWR

SEQ ID NO: 90 - protein, 24 aa, Artificial sequence
  FWR
QSVEESGGRLVTPGTPLTFSCTAS

SEQ ID NO: 91 - protein, 17 aa, Artificial sequence
  FWR
MSWVRQAPGKGLEWIGL

SEQ ID NO: 92 - protein, 36 aa, Artificial sequence
  FWR
YYASWAKGRFTISRTSTTVDLKLTSLTTEDAATYFC

SEQ ID NO: 93 - protein, 130 aa, Artificial sequence
  FWR
WGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHHAAAT

SEQ ID NO: 94 - protein, 26 aa, Artificial sequence
  FWR
DPMLTQTPSSVSAPVGGTVTIKCQAS

SEQ ID NO: 95 - protein, 17 aa, Artificial sequence
  FWR
LAWYQQKPGQRPKLLIY

SEQ ID NO: 96 - protein, 36 aa, Artificial sequence
  FWR
TLASGVSSRFKGSGSETQFTLTISDVQCDDAATYYC

SEQ ID NO: 97 - protein, 27 aa, Artificial sequence
  FWR
FGGGTEVVVKRTVGRHLSSSSRHLMSS

SEQ ID NO: 98 - protein, 25 aa, Artificial sequence
  FWR
QEQLEESGGRLVTPGTPLTLTCTVS

SEQ ID NO: 99 - protein, 17 aa, Artificial sequence
  FWR
MGWVRQAPGKGLEYIGI

SEQ ID NO: 100 - protein, 36 aa, Artificial sequence
  FWR
QYATWAKGRFTISKTSTTVDLKITSLTTEDTATYFC

SEQ ID NO: 101 - protein, 47 aa, Artificial sequence
  FWR
WGPGTLVPSPQLAPRAHRSSRWHPPPRAPLGAQRPWAAWSRTTSPNP

SEQ ID NO: 102 - protein, 26 aa, Artificial sequence
  FWR
YVMMTQTPASVSEPVGGTVTIKCQAS

SEQ ID NO: 103 - protein, 17 aa, Artificial sequence
  FWR
LAWYQQKPGQPPKLLIY

SEQ ID NO: 104 - protein, 36 aa, Artificial sequence
  FWR
TLASGVPSRFSGSGSGTQFTLTISDVECDDAATYYC

SEQ ID NO: 105 - protein, 117 aa, Artificial sequence
  FWR
FGGGTEVVVKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTESFNRGEC

SEQ ID NO: 106 - protein, 26 aa, Artificial sequence
  FWR
MAQSLEESGGRLVTPGTPLTLTCTAS

SEQ ID NO: 107 - protein, 17 aa, Artificial sequence
  FWR
VSWVRQAPGKGLEWIGH

SEQ ID NO: 108 - protein, 36 aa, Artificial sequence
  FWR
YYATWAKGRFTISRTSTTVDLKITSPTTEDTATYFC

SEQ ID NO: 109 - protein, 132 aa, Artificial sequence
  FWR
WGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHHAAATER

SEQ ID NO: 110 - protein, 26 aa, Artificial sequence
  FWR
YVMMTQTPSSTSAAVGGTVTINCQSS

SEQ ID NO: 111 - protein, 17 aa, Artificial sequence
  FWR
LSWYQQKPGQLPKLLIY

SEQ ID NO: 112 - protein, 36 aa, Artificial sequence
  FWR
TLASGVPSRFKGSGSGTQFTLTISELQCDDAATYYC

SEQ ID NO: 113 - protein, 117 aa, Artificial sequence; identical to SEQ ID NO: 65
  FWR

SEQ ID NO: 114 - protein, 24 aa, Artificial sequence
  FWR
QSVEESGGRLVTPGTPLTLTCTVS

SEQ ID NO: 115 - protein, 17 aa, Artificial sequence
  FWR
MSWVRQAPGGGLEWIGT

SEQ ID NO: 116 - protein, 37 aa, Artificial sequence
  FWR
YDASWMNGRFTISKTSSTTVDLKMTSLTTEDTATYFC

SEQ ID NO: 117 - protein, 132 aa, Artificial sequence
  FWR
WGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHHAAATER

SEQ ID NO: 118 - protein, 10 aa, Artificial sequence
  Antigen
EDALNETRES

SEQ ID NO: 119 - protein, 10 aa, Artificial sequence
  Antigen
PGVCNETMMA

SEQ ID NO: 120 - protein, 9 aa, Artificial sequence
  Antigen
EEFLNQSSP

SEQ ID NO: 121 - protein, 10 aa, Artificial sequence
  Antigen
REIRHNSTGC

SEQ ID NO: 122 - protein, 10 aa, Artificial sequence
  Antigen
CSTNNPSQAK

SEQ ID NO: 123 - protein, 10 aa, Artificial sequence
  Antigen
WKMLNTSSLE

SEQ ID NO: 124 - protein, 10 aa, Artificial sequence
  Antigen
SRLANLTQGE

SEQ ID NO: 125 - protein, 219 aa, Artificial sequence
  Recombinant protein
DPVLTQTPSPVSAAVGGTVTINCQASQSVYNNNELSWYQQKPGQPPKPLIYQASKLASGVPSRFKGSGSGTQFTLTISDLECDDAATYYCQGIYLGSDWYDVFGGGTEVVVKRTVAAPSVFIFPPSDEQLKSGTASVVCLLNNFYPREAKVQWKVDNALQSGNSQESVTEQDSKDSTYSLSSTLTLSRADYEKHKVYACEVTHQGLSSPVTKSFNRGEC

SEQ ID NO: 126 - protein, 214 aa, Artificial sequence
  Recombinant protein
MTQTPASASEPVGGTVTIKCQASQSIGNLLAWYQQKPGQPPKFLIYKASTLASGVSSQFKGSGSGTEFTLTISDLECADAATYYCQSYYAIASYGVAFGAGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 220 aa, Artificial sequence
  Recombinant protein
DVVMTQTPASVEAAVGGTVTIKCQASQSISTYLSWHQQKPGQPPKLLIYRASTLESGVPSRFKGSGSGTEFALTISDLECADAATYYCQTAHDSSGRGTWGVIFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 212 aa, Artificial sequence
  Recombinant protein
MTQTPASVEVAVGGTVTIKCQASQSINGYLAWYQQKPGQPPKLLIYQASTLASGVSSRFQGSGSGTEYTLTISGVQCDDAATYYCQGDYYGWIRTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 219 aa, Artificial sequence
  Recombinant protein
DVVMTQTPSSKSVPVGDTVTINCQASESVYVNNFLSWYRQKPGQPPKRLIYSASTLASGVPSRFSGSGSGTQFTLTISDVVCDDAATYYCAGYHEFNTDGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 219 aa, Artificial sequence
  Recombinant protein
DPMLTQTPSSVSAAVGGTVTANCQSSQSVRGNNDLAWYQQKPGQPPKLLIYDASKLASGVPSRFKGSGSGTDFTLTISDLECADAATYYCQQGHRVEDVANAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 124 aa, Artificial sequence
  Recombinant protein
DPMLTQTPSSVSAPVGGTVTIKCQASESISSRLAWYQQKPGQRPKLLIYSASTLASGVSSRFKGSGSETQFTLTISDVQCDDAATYYCLGTYSAIRTFGGGTEVVVKRTVGRHLSSSSRHLMSS

SEQ ID NO: 132 - protein, 217 aa, Artificial sequence
  Recombinant protein
YVMMTQTPASVSEPVGGTVTIKCQASQSIDSYLAWYQQKPGQPPKLLIYKASTLASGVPSRFSGSGSGTQFTLTISDVECDDAATYYCQCSHYGSNWLGPFGGGTEVVVKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTESFNRGEC

SEQ ID NO: 133 - protein, 217 aa, Artificial sequence
  Recombinant protein
YVMMTQTPSSTSAAVGGTVTINCQSSQSVYSNSFLSWYQQKPGQLPKLLIYKASTLASGVPSRFKGSGSGTQFTLTISELQCDDAATYYCQGGYVDWMRAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 227 aa, Artificial sequence
  Recombinant protein
QSLEESGGRLVTPGGSLTLTCTVSGFSLSSYAMSWVRQAPGKGLEWIGIIGVNGDTYYASWAKGRFTISKTSTTVGLKITSPTTEDTATYFCARVRYPYYDTDAFDPWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 135 - protein, 229 aa, Artificial sequence
  Recombinant protein
QEQLKESGGRLVTPGGSLTLTCTVAGFSLSRYPMNVRQAPGKGLEWIGVISSGGWLTFYANWAKGRFTISKTSTTVDLKITSPTTEDTTTYFCARFGRYGNTDYYYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 136 - protein, 225 aa, Artificial Sequence
  Recombinant protein
QEQLEESGGRLVTPGTPLTLTCAVSGFSLSSYGVSWVRQAPGKGLEYIGYIDVSGSAYYASWARGRFTISRTSTTVDLKMTSLTTEDTATYFCARGSPGYDANDLWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 137 - protein, 218 aa, Artificial sequence
  Recombinant protein
QQLEESGGRLVTPGTPLTLTCTASGMDLSKYWMTWVRQAPGKGLEYIGIIETGGSAYYASWAKGRFTISRTSTTVDLKMISPTTEDTATYFCGRWGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 138 - protein, 225 aa, Artificial sequence
  Recombinant protein
QEQLVESGGRLVTPGTPLILTCTASGFSLSRHTMSWVRQAPGKGLEWIGYITYGGSAYSANWAKGRFTISRTSTTVDLKMNSLTTEDTATYFCGRVGAYGAYYDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 139 - protein, 227 aa, Artificial sequence
  Recombinant protein
QSVEESGGRLVTPGTPLTFSCTASGFSLNSYYMSWVRQAPGKGLEWIGLVSTDGSAYYASWAKGRFTISRTSTTVDLKLTSLTTEDAATYFCARDRGSDSYIDQLDLWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 140 - protein, 151 aa, Artificial sequence
  Recombinant protein
QEQLEESGGRLVTPGTPLTLTCTVSGFSLSTYNMGWVRQAPGKGLEYIGIIYGSGSVQYATWAKGRFTISKTSTTVDLKITSLTTEDTATYFCARMGSGWGFNIWGPGTLVPSPQLAPRAHRSSRWHPPPRAPLGAQRPWAAWSRTTSPNP

SEQ ID NO: 141 - protein, 239 aa, Artificial sequence
  Recombinant protein
MAQSLEESGGRLVTPGTPLTLTCTASGFSLNTYYWVSWVRQAPGKGLEWIGHINPDGTAYYATWAKGRFTISRTSTTVDLKITSPTTEDTATYFCARLGSTGDNFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGSHHHHHHAAATER

SEQ ID NO: 142 - protein, 228 aa, Artificial sequence
  Recombinant protein
QSVEESGGRLVTPGTPLTLTCTVSGIDLSSNAMSWVRQAPGGGLEWIGTINTNGKTYDASWMNGRFTISKTSSTTVDLKMTSLTTEDTATYFCARGRWTGNTGWYIDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGS

SEQ ID NO: 143 - DNA, 1454 bp, Artificial sequence
  Recombinant polynucleotide
gaccctgtgctgacccagactccatcccctgtgtctgcagctgtgggaggcacagtcaccatcaactgccaggccagtcagagtgtttataataacaacgaattatcctggtatcagcagaaaccagggcagcctcccaagcccctgatctaccaggcatccaaactggcatctggggtcccatcgcggttcaaaggcagtggatctgggacgcagttcactctcaccatcagcgacctggagtgtgacgatgctgccacttactactgtcaaggcatttatttgggtagtgattggtacgatgttttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcagagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccagtcgctggaggagtccgggggtcgcctggtaacgcctggaggatccctgacactcacctgcacagtctctggattctccctcagtagctatgcaatgagctgggtccgccaggctccagggaaggggctggaatggatcggaatcattggtgttaatggtgacacatactacgcgagctgggcaaaaggccgattcaccatctccaaaacctcgaccacggtgggtctgaaaatcaccagtccgacaaccgaggacacggccacctatttctgtgccagagtccgttatccgtattatgatacagatgcttttgatccctggggcccgggcaccctggtcaccatctcctcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccac

SEQ ID NO: 144 - DNA, 1448 bp, Artificial sequence
  Recombinant polynucleotide
atgacccagactccagcctccgcgtctgaacctgtgggaggcacagtcaccatcaagtgccaggccagtcagagcattggcaacctcttagcctggtatcagcagaaaccagggcagcctcccaagttcctgatctacaaggcatccactctggcatctggggtctcatcgcagttcaaaggcagtggatctgggacagagttcactctcaccatcagcgacctggagtgtgccgatgctgccacttactactgtcaaagctattatgctattgctagttatggtgttgctttcggcgcagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccaggagcagctgaaggagtccgggggtcgcctggtaacgcctggaggatccctgacactcacctgcacagtcgccggattctccctcagtagatatccaatgaactaggtccgccaggctccagggaaggggctggaatggatcggagtcattagtagtggtggttggctcacattctacgcgaactgggcgaaaggccgattcaccatctccaaaacctcgaccacggtggatctgaaaatcaccagtccgacaaccgaggacacgaccacctatttttgtgccaggtttggccgctatggtaatactgattactactactttgacttgtggggtcaaggcaccctggtcaccgtctcctcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccac

SEQ ID NO: 145 - DNA, 1532 bp, Artificial sequence
  Recombinant polynucleotide
atgaaatacctattgcctacggcagccgctggattgttattactcgcagcaagcggcgcgcatgccgatgtcgtgatgacccagactccagcctccgtggaggcagctgtgggaggcacagtcaccatcaagtgccaggccagtcagagtattagtacttacttatcctggcatcagcagaaaccagggcagcctcccaagctcctgatctacagggcatccactctggaatctggggtcccgtcgcggttcaaaggcagtggatctgggacagagttcgctctcaccatcagcgacctggagtgtgccgatgctgccacttactactgtcaaactgctcatgatagtagtggtcgtggtacttggggagttattttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccaggagcagctggaggagtccgggggtcgcctggtcacgcctgggacacccctgacactcacctgcgcagtgtctggattctccctcagtagctatggagtgagctgggtccgccaggctccagggaaggggctggaatacatcggatatattgatgtaagtggtagcgcatactacgcgagctgggcaagaggccgattcaccatctccagaacctcgaccacggtggatctgaaaatgaccagtctgacaaccgaggacacggccacctatttctgtgccagaggctctcctggttatgatgccaatgacttgtggggccagggcaccctggtcaccatctcttcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccacgcggccgctacggaa

SEQ ID NO: 146 - DNA, 1406 bp, Artificial sequence
  Recombinant polynucleotide
atgacccagactccagcctccgtggaggtagctgtgggaggcacagtcaccatcaagtgccaggccagtcagagcattaatggctacttagcctggtatcagcagaaaccagggcagcctcccaagctcctgatttaccaggcttccactctggcatctggggtctcatcgcggttccaaggcagtggatctgggacagagtacactctcaccatcagcggcgtgcagtgtgacgatgctgccacttactactgtcaaggcgattattatggttggattaggactttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccagcagctggaggagtctgggggtcgcctggtcacgcctgggacacccctgacactcacctgcacagcctctggaatggacctcagtaaatactggatgacctgggtccgccaggctccagggaagggactggaatatatcggaatcattgaaactggtggtagcgcatactacgcgagctgggcaaaaggccgattcaccatctccagaacctcgaccacggtggatctgaaaatgatcagtccgacaaccgaggacacggccacctatttctgtggcagatggggggacatctggggcccaggcaccctggtcaccgtctcttcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccac

SEQ ID NO: 147 - DNA, 1526 bp, Artificial sequence
  Recombinant polynucleotide
atgaaatacctattgcctacggcagccgctggattgttattactcgcagcaagcggcgcgcatgccgatgtcgtgatgacccagactccatcttccaagtctgtccctgtgggagacacagtcaccatcaattgccaggccagtgagagtgtttatgtgaacaacttcttatcctggtatcggcagaaaccagggcagcctcccaagcgcctgatctattctgcatccactctggcatctggggtcccatcgcggtttagcggcagtggatctgggacacagttcactctcaccatcagcgatgtggtgtgtgacgatgctgccacttactactgtgcaggatatcacgaatttaatactgatggtaatgctttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccaggagcagctggtggagtccgggggtcgcctggtcacgcctgggacacccctgatactcacctgcacagcctctggattctccctcagtaggcatacaatgagttgggtccgccaggctccagggaaggggctggaatggatcggatacattacttatggtggtagcgcatactccgcgaactgggcaaaaggccgattcaccatctccagaacctcgaccacggtggatctgaaaatgaatagtctgacaaccgaggacacggccacctatttctgtggcagagttggtgcttatggtgcttactatgacttgtggggccaaggcaccctggtcaccgtctcttcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccacgcgccgctacgg

SEQ ID NO: 148 - DNA, 1535 bp, Artificial sequence
  Recombinant polynucleotide
atgaaatacctattgcctacggcagccgctggattgttattactcgcagcaagcggcgcgcatgccgaccctatgctgacccagactccatcctccgtgtctgcagctgtgggaggcacagtcaccgccaattgccagtccagtcagagtgttcgtggtaacaacgacttagcctggtatcagcagaaaccagggcagcctcccaagctcctgatctatgatgcatccaaactggcatctggggtcccatcgcggttcaaaggcagtggatctgggacagacttcactctcaccatcagcgacctggagtgtgccgatgctgccacttactactgtcaacagggtcatagggttgaggatgttgctaatgctttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacggagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccagtcggtggaggagtccgggggtcgcctggtcacgcctgggacacccctgacattcagctgcacagcctctggattctccctcaatagctactacatgagttgggtccgccaggctccagggaaggggctggaatggatcggactcgttagcactgatggtagcgcatactacgcgagctgggcaaaaggccgattcaccatctccagaacctcgaccacggtggatctgaaactgaccagtctgacaaccgaggacgcggccacctatttctgtgccagagatcgtggtagtgatagttatattgatcagttggatctctggggccagggcaccctggtcaccatctcctcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccacgcggccgctacgtaa

SEQ ID NO: 149 - DNA, 1261 bp, Artificial sequence
  Recombinant polynicleotide
atgaaatacctattgcctacggcagccgctggattgttattactcgcagcaagcggcgcgcatgtcgaccctatgctgacccagactccatcctccgtgtctgcacctgtgggaggcacagtcaccatcaagtgccaggccagtgagagcattagtagtagattagcctggtatcagcagaaaccagggcagcgtcccaagctcctgatctattctgcatccactctggcatctggggtctcatcgcggtttaaaggcagtggatctgagacccagttcactctcaccatcagcgacgtgcagtgtgacgatgctgccacttactactgtctaggcacttatagtgccatccggactttcggcggagggaccgaggtggtcgtcaaacgtacggtggggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccaggagcagctggaggagtccgggggtcgcctggtcacgcctgggacacccctgacactcacctgcacagtctctggattctccctcagtacctacaacatgggctgggtccgccaggctccagggaaggggctggaatacatcggaatcatttatggcagtggtagcgtacagtacgcgacctgggcgaaaggccgattcaccatctccaaaacctcgaccacggtggatctgaaaatcaccagtctgacaaccgaggacacggccacctatttctgtgccagaatgggtagtggctggggttttaacatctggggcccaggcaccctggtcccatctcctcagctagcaccaagggcccatcggtcttcccgctggcaccctcctccaagagcacctctgggggcacagcggccctgggctgcctggtcaaggactacttccccgaacccgtga

SEQ ID NO: 150 - DNA, 1526 bp, Artificial sequence
  Recominant polynucleotide
atgaaatacctattgcctacggcagccgctggattgttattactcgcagcaagcggcgcgcatgcctatgtcatgatgacccagactccagcctccgtgtctgaacctgtgggaggcacagtcaccatcaagtgccaggccagtcagagcattgatagctacttagcctggtatcagcagaaaccagggcagcctcccaagctcctgatctacaaggcttccactctggcatctggggtcccatcgcggttcagcggcagtggatctgggacacagttcactctcaccatcagcgacgtggagtgtgacgatgctgccacttactactgtcaatgtagtcattatggtagtaattggcttggacctttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagttcscccgtcacagagagcttcaacaggggagagtgttaagtcgactaattaattaggaggaatttaaagtgaaatacctattgcctacggcagccgctggattgttattactcgctgcccagccggccatggcccagtcgctggaggagtccgggggtcgcctggtcacgcctgggacacccctgacactcacctgcacagcctctggattctccctcaatacctactattgggtgagctgggtccgccaggctccagggaaggggctggagtggatcggacacattaatcctgatgggactgcatactacgcgacctgggcaaaaggccgattcaccatctccagaacctcgaccacggtggatctgaaaatcaccagtccgacaaccgaggacacggccacgtatttctgtgccagactgggtagtactggtgacaactttaacatctggggcccaggcaccctggtcaccatctcctcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccacgcggccgctactgaaagg

SEQ ID NO: 151 - DNA, 1535 bp, Artificial sequence
  Recombinant polynucleotide
atgaaatacctattgcctacggcagccgctggattgttattactcgcagcaagcggcgcgcatgcctatgtcatgatgacccagactccatcttccacgtctgcggctgtgggaggcacagtcaccatcaactgccagtccagtcagagtgtttatagtaacagcttcttatcctggtatcagcagaaaccagggcagcttcccaagctcctgatctacaaggcttccactctggcatctggggtcccatcgcggtttaaaggcagtggatctgggacacagttcactctcacaatcagcgaacttcagtgtgacgatgctgccacttactactgtcaaggcggttatgtcgattggatgcgtgctttcggcggagggaccgaggtggtcgtcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaagtcgactaattaattaggaggaatttaaaatgaaatacctattgcctacggcagccgctggattgttattactcgctgcccagccggccatggcccagtcggtggaggagtccgggggtcgcctggtcacgcctgggacacccctgacactcacctgcacagtctctggaatcgacctcagtagcaatgcaatgagctgggtccgccaggctccagggggggggctggagtggatcggaaccattaatactaatggtaagacatacgacgcgagctggatgaatggccgattcaccatctccaaaacctcgtcgaccacggtggatctgaaaatgaccagtctgacaaccgaggacacggccacctatttctgtgccagaggtcgatggactggtaatactggttggtatatagacttgtggggcccaggcaccctggtcaccgtctcttcagctagcaccaagggcccatcggtcttcccgctggcaccctcctccaagagcacctctgggggcacagcggccctgggctgcctggtcaaggactacttccccgaacccgtgacggtgtcgtggaactcaggtgctctgaccagcggcgtccacaccttcccggctgtcctacagtctagcggactctactccctcagcagcgtagtgaccgtgccctcttctagcttgggcacccagacctacatctgcaacgtgaatcacaagcccagcaacaccaaggtggacaagaaagttgagcccaaatcttgtgacaaaactagtggatcccatcatcaccatcaccacgcggccgctacggaaagg

SEQ ID NO: 152 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 153 - protein, 10 aa, Artificial sequence
  recombinant peptide
  Biotin (1)..(1)
  GlcNAc (6)..(6)
REIRHNSTGC

SEQ ID NO: 154 - protein, 10 aa, Artificial sequence
  recombinant peptide
  Biotin (1)..(1)
REIRHNSTGC

SEQ ID NO: 155 - protein, 10 aa, Artificial sequence
  Recombinant peptide
  Biotin (1)..(1)
  GlcNAc (5)..(5)
EDALNETRES

SEQ ID NO: 156 - protein, 10 aa, Artificial sequence
  Recombinant peptide
  Biotin (1)..(1)
EDALNETRES

SEQ ID NO: 157 - protein, 10 aa, Artificial sequence
  Recombinant peptide
  Biotin (1)..(1)
  GlcNAc (5)..(5)
PGVCNETMMA

SEQ ID NO: 158 - protein, 10 aa, Artificial sequence
  Recombinant peptide
  Biotin (1)..(1)
PGVCNETMMA

SEQ ID NO: 159 - protein, 9 aa, Artificial sequence
  Recombinant peptide
  Biotin (1)..(1)
  GlcNac (5)..(5)
EEFLNQSSP

SEQ ID NO: 160 - protein, 9 aa, Artificial sequence
  Recombinant protein
  BIotin (1)..(1)
EEFLNQSSP

SEQ ID NO: 161 - protein, 10 aa, Artificial sequence
  Recombinant peptide
  Biotin (1)..(1)
  GlcNAc (5)..(5)
SRLANLTQGE

SEQ ID NO: 162 - protein, 10 aa, Artificial sequence
  Recombinant peptide
  Biotin (1)..(1)
SRLANLTQGE

SEQ ID NO: 163 - protein, 10 aa, Artificial sequence
  Recombinant peptide
  Biotin (1)..(1)
  GlcNAc (5)..(5)
CSTNNPSQAK

SEQ ID NO: 164 - protein, 10 aa, Artificial sequence
  Recombinant peptide
  Biotin (1)..(1)
CSTNNPSQAK

SEQ ID NO: 165 - protein, 10 aa, Artificial sequence
  Recombinant peptide
  Biotin (1)..(1)
  GlcNAc (5)..(5)
WKMLNTSSLE

SEQ ID NO: 166 - protein, 10 aa, Artificial sequence
  Recombinant peptide
  Biotin (1)..(1)
WKMLNTSSLE

SEQ ID NO: 167 - protein, 20 aa, Artificial sequence
  Gycosylated peptide from Clusterin/ApoJ
TKLKELPGVCNETMMALWEE